SEQUENCE LISTING (32 sequences)

SEQ ID NO: 1 - protein, 422 aa, Homo sapiens
MRWRRAPRRSGRPGPRAQRPGSAARSSPPLPLLPLLLLLGTAALAPGAAAGNEAAPAGASVCYSSPPSVGSVQELAQRAAVVIEGKVHPQRRQQGALDRKAAAAAGEAGAWGGDREPPAAGPRALGPPAEEPLLAANGTVPSWPTAPVPSAGEPGEEAPYLVKVHQVWAVKAGGLKKDSLLTVRLGTWGHPAFPSCGRLKEDSRYIFFMEPDANSTSRAPAAFRASFPPLETGRNLKKEVSRVLCKRCALPPQLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYSTSTPFLSLPE

SEQ ID NO: 2 - protein, 372 aa, Homo sapiens
GNEAAPAGASVCYSSPPSVGSVQELAQRAAVVIEGKVHPQRRQQGALDRKAAAAAGEAGAWGGDREPPAAGPRALGPPAEEPLLAANGTVPSWPTAPVPSAGEPGEEAPYLVKVHQVWAVKAGGLKKDSLLTVRLGTWGHPAFPSCGRLKEDSRYIFFMEPDANSTSRAPAAFRASFPPLETGRNLKKEVSRVLCKRCALPPQLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFYSTSTPFLSLPE

SEQ ID NO: 3 - DNA, 1269 bp, Homo sapiens
atgagatggcgacgcgccccgcgccgctccgggcgtcccggcccccgggcccagcgccccggctccgccgcccgctcgtcgccgccgctgccgctgctgccactactgctgctgctggggaccgcggccctggcgccgggggcggcggccggcaacgaggcggctcccgcgggggcctcggtgtgctactcgtccccgcccagcgtgggatcggtgcaggagctagctcagcgcgccgcggtggtgatcgagggaaaggtgcacccgcagcggcggcagcagggggcactcgacaggaaggcggcggcggcggcgggcgaggcaggggcgtggggcggcgatcgcgagccgccagccgcgggcccacgggcgctggggccgcccgccgaggagccgctgctcgccgccaacgggaccgtgccctcttggcccaccgccccggtgcccagcgccggcgagcccggggaggaggcgccctatctggtgaaggtgcaccaggtgtgggcggtgaaagccgggggcttgaagaaggactcgctgctcaccgtgcgcctggggacctggggccaccccgccttcccctcctgcgggaggctcaaggaggacagcaggtacatcttcttcatggagcccgacgccaacagcaccagccgcgcgccggccgccttccgagcctctttcccccctctggagacgggccggaacctcaagaaggaggtcagccgggtgctgtgcaagcggtgcgccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaatag

SEQ ID NO: 4 - protein, 11 aa, Homo sapiens
VCLLTVAALPP

SEQ ID NO: 5 - protein, 15 aa, Homo sapiens
ASPVSVGSVQELVQR

SEQ ID NO: 6 - protein, 8 aa, Homo sapiens
WFVVIEGK

SEQ ID NO: 7 - protein, 9 aa, Homo sapiens
KVHEVWAAK

SEQ ID NO: 8 - protein, 6 aa, Homo sapiens
  misc_feature (5)..(5): Xaa can be any naturally occurring amino acid
DLLLXV

SEQ ID NO: 9 - protein, 13 aa, Homo sapiens
  misc_feature (12)..(12): Xaa can be any naturally occurring amino acid
LGAWGPPAFPVXY

SEQ ID NO: 10 - protein, 13 aa, Homo sapiens
  misc_feature (10)..(10): Xaa can be any naturally occurring amino acid
YIFFMEPEAXSSG

SEQ ID NO: 11 - protein, 13 aa, Homo sapiens
  misc_feature (12)..(12): Xaa can be any naturally occurring amino acid
KASLADSGEYMXK

SEQ ID NO: 12 - protein, 61 aa, Homo sapiens
SHLVKCAEKEKTFCVNGGECFMVKDLSNPSRYLCKCPNEFTGDRCQNYVMASFYKAEELYQ

SEQ ID NO: 13 - protein, 60 aa, Homo sapiens
SHLVKCAEKEKTFCVNGGECFMVKDLSNPSRYLCKCPNEFTGDRCQNYVMASFYKAEELY

SEQ ID NO: 14 - protein, 65 aa, Homo sapiens
SHLVKCAEKEKTFCVNGGECFMVKDLSNPSRYLCKCPNEFTGDRCQNYVMASFYSTSTPFLSLPE

SEQ ID NO: 15 - DNA, 198 bp, Homo sapiens
agccatcttgtcaagtgtgcagagaaggagaaaactttctgtgtgaatggaggcgagtgcttcatggtgaaagacctttcaaatccctcaagatacttgtgcaagtgcccaaatgagtttactggtgatcgctgccaaaactacgtaatggccagcttctacagtacgtccactccctttctgtctctgcctgaatag

SEQ ID NO: 16 - protein, 63 aa, Homo sapiens
SHLVKCAEKEKTFCVNGGECFMVKDLSNPSRYLCKCQPGFTGARCTENVPMKVQTQEKAEELY

SEQ ID NO: 17 - DNA, 192 bp, Homo sapiens
agccatcttgtcaagtgtgcagagaaggagaaaactttctgtgtgaatggaggcgagtgcttcatggtgaaagacctttcaaatccctcaagatacttgtgcaagtgccaacctggattcactggagcgagatgtactgagaatgtgcccatgaaagtccaaacccaagaaaaagcggaggagctctactaa

SEQ ID NO: 18 - protein, 60 aa, Homo sapiens; identical to SEQ ID NO: 13

SEQ ID NO: 19 - DNA, 183 bp, Homo sapiens
agccatcttgtcaagtgtgcagagaaggagaaaactttctgtgtgaatggaggcgagtgcttcatggtgaaagacctttcaaatccctcaagatacttgtgcaagtgcccaaatgagtttactggtgatcgctgccaaaactacgtaatggccagcttctacaaagcggaggagctctactaa

SEQ ID NO: 20 - protein, 69 aa, Homo sapiens
SHLVKCAEKEKTFCVNGGECFMVKDLSNPSRYLCKCPNEFTGDRCQNYVMASFYKHLGIEFMEKAEELY

SEQ ID NO: 21 - DNA, 210 bp, Homo sapiens
agccatcttgtcaagtgtgcagagaaggagaaaactttctgtgtgaatggaggcgagtgcttcatggtgaaagacctttcaaatccctcaagatacttgtgcaagtgcccaaatgagtttactggtgatcgctgccaaaactacgtaatggccagcttctacaagcatcttgggattgaatttatggagaaagcggaggagctctactaa

SEQ ID NO: 22 - protein, 88 aa, Homo sapiens
SHLVKCAEKEKTFCVNGGECFMVKDLSNPSRYLCKCQPGFTGARCTENVPMKVQTQEKCPNEFTGDRCQNYVMASFYSTSTPFLSLPE

SEQ ID NO: 23 - DNA, 267 bp, Homo sapiens
agccatcttgtcaagtgtgcagagaaggagaaaactttctgtgtgaatggaggcgagtgcttcatggtgaaagacctttcaaatccctcaagatacttgtgcaagtgccaacctggattcactggagcgagatgtactgagaatgtgcccatgaaagtccaaacccaagaaaagtgcccaaatgagtttactggtgatcgctgccaaaactacgtaatggccagcttctacagtacgtccactccctttctgtctctgcctgaatag

SEQ ID NO: 24 - protein, 83 aa, Homo sapiens
SHLVKCAEKEKTFCVNGGECFMVKDLSNPSRYLCKCQPGFTGARCTENVPMKVQTQEKCPNEFTGDRCQNYVMASFYKAEELY

SEQ ID NO: 25 - DNA, 252 bp, Homo sapiens
agccatcttgtcaagtgtgcagagaaggagaaaactttctgtgtgaatggaggcgagtgcttcatggtgaaagacctttcaaatccctcaagatacttgtgcaagtgccaacctggattcactggagcgagatgtactgagaatgtgcccatgaaagtccaaacccaagaaaagtgcccaaatgagtttactggtgatcgctgccaaaactacgtaatggccagcttctacaaagcggaggagctctactaa

SEQ ID NO: 26 - DNA, 498 bp, Artificial Sequence
  IgEgf pet 15 DNA sequence
catatgttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacggatcc

SEQ ID NO: 27 - protein, 162 aa, Artificial Sequence
  translated amino acid sequence of the IgEgf pet 15 clone
LPPQLKEMKSQESAAGSKLVLRCETSSEYSSLRFKWFKNGNELNRKNKPQNIKIQKKPGKSELRINKASLADSGEYMCKVISKLGNDSASANITIVESNATSTSTTGTSHLVKCAEKEKTFCVNGGECFMVKDLSNPSRYLCKCPNEFTGDRCQNYVMASFY

SEQ ID NO: 28 - protein, 198 aa, Artificial Sequence
  translated protein from pet 15b vector containing the DNA sequence of IgEgf
MGGSHHHHHHGMASMTGGTANGVGDLYDDDDKVPGSLPPQLKEMKSQESAAGSKLVLRCETSSEYSSLRFKWFKNGNELNRKNKPQNIKIQKKPGKSELRINKASLADSGEYMCKVISKLENDSASANITIVESNATSTSTTGTSHLVKCAEKEKTFCVNGGECFMVKDLSNPSRYLCKCPNEFTGDRCQNYVMASFY

SEQ ID NO: 29 - DNA, 198 bp, Artificial Sequence
  DNA sequence of NRG1b2 egf pet 15 clone
catatgagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacaaggcggaggagctgtaccagtaaggatcc

SEQ ID NO: 30 - protein, 82 aa, Artificial Sequence
  translated protein from petl5b vector containing the NRG1b2 egf DNA
MGSSHHHHHHSSGLVPRGSHMSHLVKCAEKEKTFCVNGGECFMVKDLSNPSRYLCKCPNEFTGDRCQNYVMASFYKAEELYQ

SEQ ID NO: 31 - DNA, 236 bp, Artificial Sequence
  MIS sequence
cgataactagcagcatttcctccaacgaggatcccgcaggtaagaagctacaccggccagtggccggggcccgataactagcagcatttcctccaacgaggatcccgcaggtaagaagctacaccggccagtggccggggccgtggagccgggggcatccggtgcctgagacagaggtgctcaaggcagtctccaccttttgtctcccctctgcagagagccacattctggaagtt

SEQ ID NO: 32 - DNA, 2006 bp, Homo sapiens
  misc_feature (31)..(32): n is a, c, g, or t
ggaattcctttttttttttttttttttcttnntttttttttgcccttatacctcttcgcctttctgtggttccatccacttcttccccctcctcctcccataaacaactctcctacccctgcacccccaataaataaataaaaggaggagggcaaggggggaggaggaggagtggtgctgcgaggggaaggaaaagggaggcagcgcgagaagagccgggcagagtccgaaccgacagccagaagcccgcacgcacctcgcaccatgagatggcgacgcgccccgcgccgctccgggcgtcccggcccccgggcccagcgccccggctccgccgcccgctcgtcgccgccgctgccgctgctgccactactgctgctgctggggaccgcggccctggcgccgggggcggcggccggcaacgaggcggctcccgcgggggcctcggtgtgctactcgtccccgcccagcgtgggatcggtgcaggagctagctcagcgcgccgcggtggtgatcgagggaaaggtgcacccgcagcggcggcagcagggggcactcgacaggaaggcggcggcggcggcgggcgaggcaggggcgtggggcggcgatcgcgagccgccagccgcgggcccacgggcgctggggccgcccgccgaggagccgctgctcgccgccaacgggaccgtgccctcttggcccaccgccccggtgcccagcgccggcgagcccggggaggaggcgccctatctggtgaaggtgcaccaggtgtgggcggtgaaagccgggggcttgaagaaggactcgctgctcaccgtgcgcctggggacctggggccaccccgccttcccctcctgcgggaggctcaaggaggacagcaggtacatcttcttcatggagcccgacgccaacagcaccagccgcgcgccggccgccttccgagcctctttcccccctctggagacgggccggaacctcaagaaggaggtcagccgggtgctgtgcaagcggtgcgccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaatagtaggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggaaaaaaaaaaaaaa

(19) United States
(12) Patent Application Publication
(10) Pub. No.: US 2016/0129084 A1
(43) Pub. Date: May 12, 2016

(54) Therapeutic Dosing of a Neuregulin or a Fragment Thereof for Treatment or Prophylaxis of Heart Failure

(71) Applicant: ACORDA THERAPEUTICS, INC., Ardsley, NJ (US)

(72) Inventors: Anthony O. CAGGIANO, Larchmont, NY (US); Anindita GANGULY, Ardsley, NY (US); Jennifer IACI, Boonton, NJ (US); Tom PARRY, Hellertown, PA (US)

(21) Appl. No.: 14/772,205

(22) PCT Filed: Mar. 6, 2014
(86) PCT No.: PCT/US14/21446

§ 371 Date: Sep. 2, 2015

Related U.S. Application Data
(60) Provisional application No. 61/773,538, filed on Mar. 6, 2013.
(60) Provisional application No. 61/774,553, filed on Mar. 7, 2013.
(60) Provisional application No. 61/900,142, filed on Nov. 5, 2013.

(51) Int. Cl. A61K 38/18; A61K 31/5513; A61K 9/00

(52) U.S. Cl. CPC A61K 38/1883; A61K 38/1808; A61K 9/0019; A61K 31/5513

(57) ABSTRACT

The invention relates to treatment and prevention of heart failure in a mammal. The invention provides a dosing regimen whereby the therapeutic benefits conferred by administration of peptide comprising an epidermal growth factor-like domain, e.g., a neuregulin such as glial growth factor 2 (GGF2) or a functional fragment thereof, are maintained and/or enhanced, while concomitantly minimizing any potential side effects.

### RELATED APPLICATIONS

This application claims the benefit of, and priority to U.S. Provisional Application No. 61/773,538, filed Mar. 6, 2013, U.S. Provisional Application No. 61/774,553, filed Mar. 7, 2013, and U.S. Provisional Application No. 61/900,142, filed Nov. 5, 2013. The contents of each of these applications are hereby incorporated by reference in their entirety.

### FIELD OF THE DISCLOSURE

The field of the disclosure relates to treatment of heart failure. More specifically, the disclosure is directed to an improved dosing regimen whereby the therapeutic benefits of administration of a peptide comprising an epidermal growth factor-like (EGF-like) domain, e.g., a neuregulin, such as glial growth factor 2 (GGF2) or fragment thereof, are maintained and/or enhanced, while minimizing any potential side effects.

### BACKGROUND OF THE DISCLOSURE

A fundamental challenge associated with the administration of medications to patients in need thereof is the relationship between tolerability and efficacy. The therapeutic index is the range between which an efficacious dose of a substance can be administered to a patient and a dose at which undesired side effects to the patient are noted. Generally, the larger the difference between the efficacious dose and the dose at which side effects initiate, the more benign the substance and the more likely it is to be tolerated by the patient.

Heart failure, particularly congestive heart failure (CHF), is one of the leading causes of death in industrialized nations. Factors that underlie congestive heart failure include high blood pressure, ischemic heart disease, exposure to cardiotoxic compounds such as the anthracycline antibiotics, radiation exposure, physical trauma and genetic defects associated with an increased risk of heart failure. Thus, CHF often results from an increased workload on the heart due to hypertension, damage to the myocardium from chronic ischemia, myocardial infarction, viral disease, chemical toxicity, radiation and other diseases such as scleroderma. These conditions result in a progressive decrease in the heart's pumping ability. Initially, the increased workload that results from high blood pressure or loss of contractile tissue induces compensatory cardiomyocyte hypertrophy and thickening of the left ventricular wall, thereby enhancing contractility and maintaining cardiac function. Over time, however, the left ventricular chamber dilates, systolic pump function deteriorates, cardiomyocytes undergo apoptotic cell death, and myocardial function progressively deteriorates.

Neuregulins (NRGs) and NRG receptors comprise a growth factor-receptor tyrosine kinase system for cell-cell signaling that is involved in organogenesis and cell development in nerve, muscle, epithelia, and other tissues (Lemke, Mol. Cell. Neurosci. 7:247-262, 1996 and Burden et al., Neuron 18:847-855, 1997). The NRG family consists of four genes that encode numerous ligands containing epidermal growth factor (EGF)-like, immunoglobulin (Ig), and other recognizable domains. Numerous secreted and membrane-attached isoforms function as ligands in this signaling system. The receptors for NRG ligands are all members of the EGF receptor (EGFR) family, and include EGFR (or ErbB1), ErbB2, ErbB3, and ErbB4, also known as HER1 through HER4, respectively, in humans (Meyer et al., Development 124:3575-3586, 1997; Orr-Urtreger et al., Proc. Natl. Acad. Sci. USA 90: 1867-71, 1993; Marchionni et al., Nature 362:312-8, 1993; Chen et al., J. Comp. Neurol. 349:389-400, 1994; Corfas et al., Neuron 14:103115, 1995; Meyer et al., Proc. Natl. Acad. Sci. USA 91:1064-1068, 1994; and Pinkas-Kramarski et al., Oncogene 15:2803-2815, 1997).

The four NRG genes, NRG-1, NRG-2, NRG-3, and NRG-4, map to distinct chromosomal loci (Pinkas-Kramarski et al., Proc. Natl. Acad. Sci. USA 91:9387-91, 1994; Carraway et al., Nature 387:512-516, 1997; Chang et al., Nature 387:509-511, 1997; and Zhang et al., Proc. Natl. Acad. Sci. USA 94:9562-9567, 1997), and collectively encode a diverse array of NRG proteins. The gene products of NRG-1, for example, comprise a group of approximately 15 distinct structurally-related isoforms (Lemke, Mol. Cell. Neurosci. 7:247-262, 1996 and Peles and Yarden, BioEssays 15:815-824, 1993). The first-identified isoforms of NRG-1 included Neu Differentiation Factor (NDF; Peles et al., Cell 69, 205-216, 1992 and Wen et al., Cell 69, 559-572, 1992), heregulin (HRG; Holmes et al., Science 256:1205-1210, 1992), Acetylcholine Receptor Inducing Activity (ARIA; Falls et al., Cell 72:801-815, 1993), and the glial growth factors GGFI, GGF2, and GGF3 (Marchionni et al. Nature 362:312-8, 1993).

The NRG-2 gene was identified by homology cloning (Chang et al., Nature 387:509-512, 1997; Carraway et al., Nature 387:512-516, 1997; and Higashiyama et al., J. Biochem. 122:675-680, 1997) and through genomic approaches (Busfield et al., Mol. Cell. Biol. 17:4007-4014, 1997). NRG-2 cDNAs are also known as Neural- and Thymus-Derived Activator of ErbB Kinases (NTAK; Genbank Accession No. AB005060), Divergent of Neuregulin (Don-1), and Cerebellum-Derived Growth Factor (CDGF; PCT application WO 97/09425). Experimental evidence shows that cells expressing ErbB4 or the ErbB2/ErbB4 combination are likely to show a particularly robust response to NRG-2 (Pinkas-Kramarski et al., Mol. Cell. Biol. 18:6090-6101, 1998). The NRG-3 gene product (Zhang et al., supra) is also known to bind and activate ErbB4 receptors (Hijazi et al., Int. J. Oncol. 13:1061-1067, 1998).

An EGF-like domain is present at the core of all forms of NRGs, and is required for binding and activating ErbB receptors. Deduced amino acid sequences of the EGF-like domains encoded in the three genes are approximately 30-40% identical (pairwise comparisons). Further, there appear to be at least two sub-forms of EGF-like domains in NRG-1 and NRG-2, which may confer different bioactivities and tissue-specific potencies.

Cellular responses to NRGs are mediated through the NRG receptor tyrosine kinases EGFR, ErbB2, ErbB3, and ErbB4 of the epidermal growth factor receptor family. High-affinity binding of all NRGs is mediated principally via either ErbB3 or ErbB4. Binding of NRG ligands leads to dimerization with other ErbB subunits and transactivation by phosphorylation on specific tyrosine residues. In certain experimental settings, nearly all combinations of ErbB receptors appear to be capable of forming dimers in response to the binding of NRG-1 isoforms. However, it appears that ErbB2 is a preferred dimerization partner that may play an important role in stabilizing the ligand-receptor complex. ErbB2 does not bind ligand on its own, but must be heterologously paired with one of the other receptor subtypes. ErbB3 does possess tyrosine kinase activity, but is a target for phosphorylation by the other receptors. Expression of NRG-1, ErbB2, and ErbB4 is known to be necessary for trabeculation of the ventricular myocardium during mouse development.

Neuregulins stimulate compensatory hypertrophic growth and inhibit apoptosis of myocardiocytes subjected to physiological stress. In accordance with these observations, administration of an EGF-like domain-containing peptide, e.g., a neuregulin, such as glial growth factor 2, or a fragment thereof, is useful for preventing, minimizing, delaying the progression of, or reversing congestive heart disease resulting from underlying factors such as hypertension, ischemic heart disease, and cardiotoxicity. See, e.g., U.S. Pat. No. 6,635,249, which is incorporated herein in its entirety.

In view of the high prevalence of heart failure in the general population, there continues to be an ongoing need for additional and/or improved therapies to prevent or minimize/delay progression of this disease, such as by inhibiting loss of cardiac function or by improving cardiac function.

### SUMMARY OF THE DISCLOSURE

The present invention provides a method for treating, preventing, or delaying the progression of heart failure in a subject in need thereof comprising administering to the subject a therapeutically effective amount of a peptide, wherein the peptide comprises an epidermal growth factor-like (EGF-like) domain, e.g., a neuregulin, such as glial growth factor 2 (GGF) or a functional fragment thereof, wherein the therapeutically effective amount is from about 0.005 mg/kg bodyweight to about 4 mg/kg bodyweight, and wherein the peptide is administered on a dosing interval of at least 24 hours. In some examples, the present invention also provides a peptide comprising an epidermal growth factor-like (EGF-like) domain, e.g., a neuregulin, such as GGF2 or a functional fragment thereof, for use in a method of treating or preventing heart failure in a subject, wherein the method comprises administering the peptide in an amount of about 0.005 mg/kg to about 4 mg/kg of bodyweight of the subject at dosing intervals of at least 24 hours. For example, the dosing interval is at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

The present invention also features a method for treating, preventing, or delaying the progression of heart failure in a subject in need thereof comprising administering to the subject a peptide comprising an EGF-like domain, e.g., a neuregulin, such as GGF2 or a functional fragment thereof, according to an escalating dosing regimen, the method comprising administering the peptide at a first therapeutically effective dose, and subsequently administering a second therapeutically effective dose, wherein the second dose is higher than the first dose. In some examples, the present invention also provides a peptide comprising an EGF-like domain, e.g., a neuregulin, such as GGF2 or a functional fragment thereof, for use in a method of treating or preventing heart failure in a subject, wherein the method comprises administering the peptide at a first therapeutically effective dose, and subsequently administering a second therapeutically effective dose, wherein the second dose is higher than the first dose. In some cases, the method further comprises administering one or more subsequent therapeutically effective doses following the second dose. For example, the second or subsequent therapeutically effective dose is the same as the second dose or the previous dose. In some examples, an initial dose of the peptide is the same as one or more subsequent doses of the peptide.

In other embodiments, the invention provides a method for treating, preventing, or delaying the progression of heart failure in a subject in need thereof comprising administering to the subject a peptide comprising an EGF-like domain, e.g., a neuregulin, such as GGF2 or a functional fragment thereof, according to a dosing regimen, the method comprising administering the peptide at a first therapeutically effective dose, and subsequently administering a second therapeutically effective dose, wherein the second dose is lower than the first dose. In some cases, the method further comprises administering one or more subsequent therapeutically effective doses following the second dose. For example, the second or subsequent therapeutically effective dose is the same as the second dose or a previous dose.

In some embodiments, the therapeutically effective amount of a peptide of the invention is from about 0.007 mg/kg bodyweight to about 1.5 mg/kg bodyweight. For example, the therapeutically effective amount of the peptide is selected from the group consisting of: about 0.007 mg/kg bodyweight, about 0.02 mg/kg bodyweight, about 0.06 mg/kg bodyweight, about 0.19 mg/kg bodyweight, about 0.38 mg/kg bodyweight, about 0.76 mg/kg bodyweight, and about 1.51 mg/kg bodyweight. For example, the therapeutically effective amount of the peptide is 0.007 mg/kg bodyweight, 0.021 mg/kg bodyweight, 0.063 mg/kg bodyweight, 0.189 mg/kg bodyweight, 0.375 mg/kg bodyweight, 0.756 mg/kg bodyweight, or 1.512 mg/kg bodyweight.

For example, a therapeutically effective amount of a peptide described herein is about 0.007 mg/kg bodyweight, about 0.02 mg/kg bodyweight, about 0.06 mg/kg bodyweight, about 0.19 mg/kg bodyweight, about 0.38 mg/kg bodyweight, about 0.76 mg/kg bodyweight, or about 1.51 mg/kg bodyweight, and is administered on a dosing interval of at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, e.g., at least 90 days.

In some embodiments, the dosing interval used in a method of the invention is greater than 4 months. For example, the dosing interval is greater than 4 months, 5 months, 6 months, 7 months, 8 months, 9 months, 10 months, 11 months, 12 months, or longer. In other examples, the dosing interval is at least 2 weeks, e.g., at least 2 weeks, 3 weeks, or 4 weeks.

In some embodiments, the therapeutically effective amount of a peptide described herein is about 0.35 mg/kg bodyweight to about 3.5 mg/kg bodyweight and the dosing interval is at least 2 weeks. For example, the therapeutically effective amount of a peptide described herein is 3.5 mg/kg, 1.75 mg/kg, 0.875 mg/kg, or 0.35 mg/kg. For example, a therapeutically effective amount of the peptide of 3.5 mg/kg, 1.75 mg/kg, 0.875 mg/kg, or 0.35 mg/kg is administered via intravenous injection or infusion, e.g., to prevent, treat, or delay the progression of heart failure.

In some embodiments, the therapeutically effective amount of a peptide described herein, e.g., a neuregulin, such as GGF2 or a functional fragment thereof, is about 0.06 mg/kg bodyweight to about 0.38 mg/kg bodyweight and the dosing interval is at least 2 weeks, e.g., at least 2 weeks, 3 weeks, 4 weeks, 1 month, 2 months, 3 months (quarterly), 4 months, 5 months, 6 months, 7 months, 8 months, 9 months, 10 months, 11 months, 12 months, or longer. For example, the therapeutically effective amount of a peptide described herein is about 0.063 mg/kg, about 0.189 mg/kg, or about 0.375 mg/kg. For example, a therapeutically effective amount of the peptide of about 0.063 mg/kg, about 0.189 mg/kg, or about 0.375 mg/kg is administered via intravenous injection or infusion, e.g., to prevent, treat, or delay the progression of heart failure.

In some embodiments, a dosing regimen, e.g., escalating dosing regimen, used in accordance with a method of the invention comprises the steps of:
a) administering an initial dose of the peptide in the range of about 0.005 mg/kg to about 1.5 mg/kg, e.g., about 0.005 mg/kg bodyweight to about 0.015 mg/kg bodyweight, or about 0.007 mg/kg, about 0.021 mg/kg, about 0.063 mg/kg, about 0.189 mg/kg, about 0.378 mg/kg, about 0.756 mg/kg, or about 1.512 mg/kg;
b) thereafter administering a second dose of the peptide that is 2-fold to 3-fold above the previous dose; and
c) repeating step b) until a maximum therapeutic dose is reached,

wherein the maximum therapeutic dose does not elicit an adverse event in the subject, and wherein the doses are administered on an interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer.

In some cases, the maximum therapeutic dose is about 0.7 mg/kg bodyweight to about 1.5 mg/kg bodyweight, e.g., 0.756 mg/kg bodyweight or 1.512 mg/kg bodyweight.

In some examples, the escalating dosing method further comprises step d) continuing to administer the maximum therapeutic dose at an interval of at least 24 hours. For example, the interval and/or the period of time is at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer.

Alternatively or in addition, the method comprises a step of decreasing the dose over a period of time to a final dose of 0 mg/kg. For example, the period of time is over the course of at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer.

In some embodiments, the peptide used in any method of the invention comprises glial growth factor 2 (GGF2) or a functional fragment thereof. For example, the GGF2 or functional fragment thereof comprises the amino acid sequence of SEQ ID NO: 1 or SEQ ID NO: 2.

The invention provides methods to treat, prevent, or delay the progression of heart failure, e.g., chronic heart failure in a subject in need thereof. For example, the subject has suffered from chronic heart failure for at least 1 month, e.g., at least 1, 2, 3, 4, 5, 6, or more months, prior to administration of the peptide. In other examples, the subject suffers from class 2, 3, or 4 heart failure prior to administration of the peptide. In some embodiments, the subject has a left ventricular ejection fraction of 40% or less, e.g., 10-40%, or 40%, 35%, 30%, 25%, 20%, 15%, 10%, or less, prior to administration of the peptide.

In yet other embodiments, the subject suffers from heart failure with preserved ejection fraction. For example, the subject suffers from heart failure which exhibits no significant decrease in left ventricular ejection fraction (LVEF) compared to normal LVEF levels prior to administration of the peptide. In a further embodiment, the subject suffers from heart failure with reduced ejection fraction. By way of example and without limitation, the LVEF is less than 60% and greater than 40%, e.g., about 45-55%, or about 45%, 46%, 47%, 48%, 49%, 50%, 51%, 52%, 53%, 54%, or about 55%.

According to the methods of the invention a therapeutically effective amount of a peptide described herein is sufficient to increase the left ventricular ejection fraction (LVEF), decrease the end systolic volume (ESV), decrease the end diastolic volume (EDV), increase the fractional shortening (FS), or a combination thereof, in the subject. For example, the increase in the left ventricular ejection fraction (LVEF), the decrease in the end systolic volume (ESV), the decrease in the end diastolic volume (EDV), the increase in the fractional shortening (FS), or combination thereof occurs within 90 days, e.g., within 2 weeks, 3 weeks, 4 weeks, 5 weeks, or more of the first administration of the peptide. In some examples, the therapeutically effective amount of the peptide is sufficient to maintain or stabilize the LVEF, ESV, FS, and/or EDV, or combinations thereof in the subject, e.g., for the periods of time described above.

For example, a therapeutically effective amount of a peptide described herein is sufficient to increase the LVEF of the subject by at least 1-20%. In some cases, a therapeutically effective amount of a peptide described herein is sufficient to increase the LVEF of the subject in need thereof to an ejection fraction of about 10-40%, e.g., the LVEF of the subject is increased to an ejection fraction of about 10%, 15%, 20%, 25%, 30%, 35%, or about 40%. In other cases, a therapeutically effective amount of a peptide described herein is sufficient to increase the LVEF of the subject in need thereof to an ejection fraction of about 40-60%, e.g., the LVEF of the subject is increased to an ejection fraction of about 40%, 45%, 50%, 55%, or about 60%. In yet other cases, a therapeutically effective amount of a peptide described herein is sufficient to completely restore the LVEF of the subject in need thereof to a normal LVEF value. In some cases, this increase in LVEF occurs within 10, 20, 30, 40, 50, 60, 70, 80, or 90 days of the first administration of the peptide.

In other examples, a therapeutically effective amount of a peptide described herein is sufficient to decrease the EDV of the subject by at least 1-60 mL. In some cases, this decrease in EDV occurs within 10, 20, 30, 40, 50, 60, 70, 80, or 90 days of the first administration of the peptide.

In some embodiments, a therapeutically effective amount of a peptide described herein is sufficient to decrease the ESV of the subject by at least 1-30 mL. In some cases, this decrease in ESV occurs within 10, 20, 30, 40, 50, 60, 70, 80, or 90 days of the first administration of the peptide.

In other embodiments, a therapeutically effective amount of a peptide described herein is sufficient to increase the FS of the subject by at least 1-15%. In some cases, a therapeutically effective amount of a peptide described herein is sufficient to increase the FS of the subject tin need thereof to a Percent Fractional Shortening of about 15%, e.g., about 1%, 2%, 3%, 4%, 6%, 7%, 8%, 9%, 10%, or about 15%. In other cases a therapeutically effective amount of a peptide described herein is sufficient to increase the FS of the subject in need thereof to a Percent Fractional Shortening of about 15-20%, e.g., about 15%, 16%, 17%, 18%, 19%, or about 20%. In yet other cases a therapeutically effective amount of a peptide described herein is sufficient to increase the FS of the subject in need thereof to a Percent Fractional Shortening of about 20-25%, e.g., about 20%, 21%, 22%, 23%, 24%, or about 25%. In further cases, a therapeutically effective amount of a peptide described herein is sufficient to increase the FS of the subject in need thereof to a Percent Fractional Shortening of about 25-45%, e.g., about 25%, 26%, 27%, 28%, 29%, 30%, 31%, 32%, 33%, 34%, 35%, 36%, 37%, 38%, 39%, 40%, 41%, 42%, 43%, 44%, or about 45%. In some cases, the increase in FS occurs within 1, 2, 3, 4, 5, 6, 7, 8, 9, 10, 11, 12, 13, 14, or 15 weeks of the first administration of the peptide.

In some embodiments of the invention, a peptide described herein is administered intravenously or subcutaneously.

In other embodiments, a method of the invention further comprises administering a therapeutically effective amount of a benzodiazepine, e.g., midazolam, to the subject. For example, the therapeutically effective amount of benzodiazepine is administered prior to, simultaneously with, or following the first administration of a therapeutically effective amount of a peptide described herein. In some embodiments, the benzodiazepine and the peptide of the invention are co-formulated in a single composition. In other examples, the benzodiazepine and the peptide of the invention are formulated separately, e.g., in two separate compositions.

### BRIEF DESCRIPTION OF THE DRAWINGS

FIG. 1 is a line graph depicting the half-life of recombinant human GGF2 (rhGGF2) following iv administration.

FIG. 2 is a line graph depicting the half-life of recombinant human GGF2 (rhGGF2) following subcutaneous administration.

FIG. 3 is a set of two schematics of the pSV-AHSG and pCMGGF2 plasmids.

FIG. 4 is a schematic showing the placement of the GGF2 coding sequence after the EBV BMLF-1 intervening sequence (MIS) in the expression vector.

FIG. 5 is a histogram depicting cardiac function as exemplified by changes in Ejection Fraction and Fractional Shortening. As indicated, rats were treated with GGF2 at 0.625 mg/kg or an equimolar amount of an EGF-like fragment (fragment; EGF-id) intravenously (iv) everyday (q day).

FIG. 6 is a line graph depicting cardiac function as revealed by changes in Ejection Fraction and Fractional Shortening. As indicated, rats were treated with GGF2 at 0.625 mg/kg or 3.25 mg/kg iv q day.

FIG. 7 shows a line graph depicting cardiac function as revealed by significant improvement in end systolic volume during the treatment period. As indicated, rats were treated with GGF2 at 0.625 mg/kg or 3.25 mg/kg iv q day.

FIG. 8 is a line graph depicting cardiac function as revealed by changes in Ejection Fraction and Fractional Shortening. As indicated, rats were treated with GGF2 3.25 mg/kg intravenously (iv) q24, 48 or 96 hours.

FIG. 9 is a line graph depicting cardiac function as revealed by changes in the echocardiographic ejection fraction. As indicated, rats were treated with vehicle or GGF2 3.25 mg/kg intravenously (iv), with or without BSA.

FIG. 10 is a schematic diagram of a decision tree for GGF2 dose continuation and/or escalation as described in Example 4.

FIG. 11 is a graph showing the mean change in LVEF (AEF) over time (days) following a single infusion of GGF2 or Placebo.

FIG. 12 is a schematic outlining the echocardiography protocol used in the dose escalation study of GGF2. (PBO=placebo; DLT=dose limiting toxicity).

FIG. 13 is a series of echocardiograms showing the change in LVEF over time (days) following a single infusion of either the highest dose of GGF2 (1.512 mg/kg) or Placebo.

FIG. 14 is a pair of graphs showing the mean change in dimensions (A volume) over time (days) following a single infusion of Placebo or GGF2. The graph on left panel depicts the change in end-diastolic volume (EDV) as a function of time (measured in days post-treatment). The graph on right panel depicts the change in end-systolic volume (ESV) as a function of time (measured in days post-treatment).

FIG. 15 is a graph showing the effects of various dose levels of GGF2 on ejection fraction. Shown are mean ejection fractions following intravenous administration of GGF2. Data are presented as mean±SEM. n=12/14 per group.

FIG. 16 is a graph showing the effects of various dose levels of GGF2 on the net change in ejection fraction from baseline. Data are presented as mean±SEM. n=12/14 per group.

FIG. 17 is a graph showing the effects of various dose levels of GGF2 on % FS. Shown are mean % FS following intravenous administration of GGF2. Data are presented as mean±SEM. n=12/14 per group.

FIG. 18 is a graph showing the effects of various dose levels of GGF2 on the net change in fractional shortening from baseline. Data are presented as mean±SEM. n=12/14 per group.

FIG. 19 is a graph showing the effects of various dose levels of GGF2 on end systolic volume (ESV). Shown are mean ESVs following intravenous of GGF2. Data are presented as mean±SEM. n=9/14 per group.

FIG. 20 is a graph showing the effects of various dose levels of GGF2 on end diastolic volume (EDV). Shown are mean EDVs following intravenous of GGF2. Data are presented as mean±SEM. n=9/14 per group.

FIG. 21 is a graph showing the effects of various dose levels of GGF2 on ventricular mass. Data are presented as mean±SEM. n=9/14 per group.

FIG. 22 is a graph showing the effects of various dose levels of GGF2 on body weight. Shown are mean body weights (g) of all groups over time. Data are presented as mean±SEM. n=9/14 per group.

FIG. 23 is a graph showing the effects of various dose levels of GGF2 on heart weights. Shown are mean heart weights (g) of all groups over time. Data are presented as mean±SEM. n=9/14 per group.

### DETAILED DESCRIPTION OF THE DISCLOSURE

The present inventors made the discovery that discontinuous or intermittent administration of an EGF-like domain-containing peptide, e.g., a neuregulin, such as glial growth factor 2 (GGF2), or a fragment thereof, at appropriately spaced time intervals delivers a therapeutically effective amount of the EGF-like domain-containing peptide to a patient in need thereof and such a treatment regimen is useful for preventing, prophylaxing, delaying the progression of, ameliorating, minimizing, treating or reversing heart disease, such as congestive heart failure.

The present disclosure provides a method for treating, preventing, or delaying the progression of heart failure in a subject by providing a peptide comprising an epidermal growth factor-like (EGF-like) domain, e.g., a neuregulin, such as GGF2, or functional fragment thereof.

Neuregulins (NRGs) are growth factors related to epidermal growth factors that bind to erbB receptors. They have been shown to improve cardiac function in multiple models of heart failure, cardiotoxicity and ischemia. NRGs have also been shown to protect the nervous system in models of stroke, spinal cord injury, nerve agent exposure, peripheral nerve damage and chemotoxicity.

There are four NRG genes (NRG-1, NRG-2, NRG-3, and NRG-4). Peptides encoded by the NRG-1, NRG-2, NRG-3 and NRG-4 genes possess EGF-like domains that allow them to bind to and activate ErbB receptors. Holmes et al. (Science 256:1205-1210, 1992) have shown that the EGF-like domain alone is sufficient to bind and activate the p185erbB2 receptor. Accordingly, any peptide product encoded by the NRG-1, NRG-2, NRG-3, or NRG-4 gene, or any neuregulin-like peptide, e.g., a peptide having an EGF-like domain encoded by a neuregulin gene or cDNA (e.g., an EGF-like domain containing the NRG-1 peptide subdomains C-C/D or C-C/D′, as described in U.S. Pat. No. 5,530,109, U.S. Pat. No. 5,716,930, and U.S. Pat. No. 7,037,888; or an EGF-like domain as disclosed in WO 97/09425) can be used in the methods of the disclosure to prevent, treat, or delay the progression of heart failure, e.g., congestive heart failure. The contents of each of U.S. Pat. No. 5,530,109; U.S. Pat. No. 5,716,930; U.S. Pat. No. 7,037,888; and WO 97/09425 are incorporated herein in its entirety.

In some embodiments, the neuregulin is the gene, gene product or respective subsequence or fragment thereof comprising, consisting essentially of, or consisting of: NRG-1, NRG-2, NRG-3 or NRG-4. In a preferred embodiment, an NRG subsequence or functional fragment thereof comprises an epidermal growth factor-like (EGF-like) domain or a homologue thereof. A peptide homologue to an EGF-like domain peptide is determined by finding structural homology or by the homologue peptide performing as an EGF-like peptide does in functional assays such as by binding and activating ErbB receptors. A functional fragment of an NRG binds to and activates an ErbB receptor. Preferably the functional fragment of an NRG is at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 90, 95, 100, 110, 120, 130, 140, 150, 160, 170, 180, 190, 200, 220, 240, 260, 280, 300, 320, 340, 360, 380, 400, or 420 amino acids long.

In some embodiments, a peptide used in the methods of the invention is glial growth factor 2 (GGF2), e.g., recombinant human GGF2, or a functional fragment thereof. A functional fragment of GGF2 binds to and activates an ErbB receptor and comprises 422 amino acids or less, e.g., 422, 420, 418, 416, 414, 412, 410, 408, 406, 404, 402, 400, 398, 396, 394, 392, 390, 388, 386, 384, 382, 380, 379, 378, 377, 376, 375, 374, 373, 372, 371, 370, 369, 368, 367, 366, 365, 360, 355, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 55, 50, 45, 40, 35, 30, 25, 20 amino acids, or less, of SEQ ID NO: 1. For example, a functional fragment of GGF2 comprises 372 amino acids of SEQ ID NO: 1. Preferably, a functional fragment of GGF2 comprises the amino acid sequence of SEQ ID NO: 2.

In some examples, a nucleic acid sequence, e.g., a cDNA, such as clone GGF2HBS5 (see, e.g., U.S. Pat. No. 5,530,109, incorporated herein by reference), contains a coding sequence for human full length GGF2 and comprises the following sequence:

(SEQ ID NO: 32)
ggaattcctt tttttttttt tttttttctt nntttttttt tgcccttata cctcttcgcc tttctgtggt
tccatccact tcttccccct cctcctccca taaacaactc tcctacccct gcacccccaa taaataaata
aaaggaggag ggcaaggggg gaggaggagg agtggtgctg cgaggggaag gaaaagggag gcagcgcgag
aagagccggg cagagtccga accgacagcc agaagcccgc acgcacctcg cacc
atgagatgg cgacgcgccc cgcgccgctc cgggcgtccc
ggcccccggg cccagcgccc cggctccgcc gcccgctcgt cgccgccgct gccgctgctg
ccactactgc tgctgctggg gaccgcggcc ctggcgccgg gggcggcggc cggcaacgag
gcggctcccg cgggggcctc ggtgtgctac tcgtccccgc ccagcgtggg atcggtgcag
gagctagctc agcgcgccgc ggtggtgatc gagggaaagg tgcacccgca gcggcggcag
cagggggcac tcgacaggaa ggcggcggcg gcggcgggcg aggcaggggc gtggggcggc
gatcgcgagc cgccagccgc gggcccacgg gcgctggggc cgcccgccga ggagccgctg
ctcgccgcca acgggaccgt gccctcttgg cccaccgccc cggtgcccag cgccggcgag
cccggggagg aggcgcccta tctggtgaag gtgcaccagg tgtgggcggt gaaagccggg
ggcttgaaga aggactcgct gctcaccgtg cgcctgggga cctggggcca ccccgccttc
ccctcctgcg ggaggctcaa ggaggacagc aggtacatct tcttcatgga gcccgacgcc
aacagcacca gccgcgcgcc ggccgccttc cgagcctctt tcccccctct ggagacgggc
cggaacctca agaaggaggt cagccgggtg ctgtgcaagc ggtgcgcctt gcctccccaa
ttgaaagaga tgaaaagcca ggaatcggct gcaggttcca aactagtcct tcggtgtgaa
accagttctg aatactcctc tctcagattc aagtggttca agaatgggaa tgaattgaat
cgaaaaaaca aaccacaaaa tatcaagata caaaaaaagc cagggaagtc agaacttcgc
attaacaaag catcactggc tgattctgga gagtatatgt gcaaagtgat cagcaaatta
ggaaatgaca gtgcctctgc caatatcacc atcgtggaat caaacgctac atctacatcc
accactggga caagccatct tgtaaaatgt gcggagaagg agaaaacttt ctgtgtgaat
ggaggggagt gcttcatggt gaaagacctt tcaaacccct cgagatactt gtgcaagtgc
ccaaatgagt ttactggtga tcgctgccaa aactacgtaa tggccagctt ctacagtacg
tccactccct ttctgtctct gcctgaatag
taggagcatg ctcagttggt gctgctttct tgttgctgca tctcccctca gattccacct agagctagat
gtgtcttacc agatctaata ttgactgcct ctgcctgtcg catgagaaca ttaacaaaag caattgtatt
acttcctctg ttcgcgacta gttggctctg agatactaat aggtgtgtga ggctccggat gtttctggaa
ttgatattga atgatgtgat acaaattgat agtcaatatc aagcagtgaa atatgataat aaaggcattt
caaagtctca cttttattga taaaataaaa atcattctac tgaacagtcc atcttcttta tacaatgacc
acatcctgaa aagggtgttg ctaagctgta accgatatgc acttgaaatg atggtaagtt aattttgatt
cagaatgtgt tatttgtcac aaataaacat aataaaagga aaaaaaaaaa aaa

where n = any nucleotide

The nucleic acid, e.g., cDNA, coding sequence for full length human GGF2 is provided below:

(SEQ ID NO: 3)
atgagatgg cgacgcgccc cgcgccgctc cgggcgtccc
ggcccccggg cccagcgccc cggctccgcc gcccgctcgt cgccgccgct gccgctgctg
ccactactgc tgctgctggg gaccgcggcc ctggcgccgg gggcggcggc cggcaacgag
gcggctcccg cgggggcctc ggtgtgctac tcgtccccgc ccagcgtggg atcggtgcag
gagctagctc agcgcgccgc ggtggtgatc gagggaaagg tgcacccgca gcggcggcag
cagggggcac tcgacaggaa ggcggcggcg gcggcgggcg aggcaggggc gtggggcggc
gatcgcgagc cgccagccgc gggcccacgg gcgctggggc cgcccgccga ggagccgctg
ctcgccgcca acgggaccgt gccctcttgg cccaccgccc cggtgcccag cgccggcgag
cccggggagg aggcgcccta tctggtgaag gtgcaccagg tgtgggcggt gaaagccggg
ggcttgaaga aggactcgct gctcaccgtg cgcctgggga cctggggcca ccccgccttc
ccctcctgcg ggaggctcaa ggaggacagc aggtacatct tcttcatgga gcccgacgcc
aacagcacca gccgcgcgcc ggccgccttc cgagcctctt tcccccctct ggagacgggc
cggaacctca agaaggaggt cagccgggtg ctgtgcaagc ggtgcgcctt gcctccccaa
ttgaaagaga tgaaaagcca ggaatcggct gcaggttcca aactagtcct tcggtgtgaa
accagttctg aatactcctc tctcagattc aagtggttca agaatgggaa tgaattgaat
cgaaaaaaca aaccacaaaa tatcaagata caaaaaaagc cagggaagtc agaacttcgc
attaacaaag catcactggc tgattctgga gagtatatgt gcaaagtgat cagcaaatta
ggaaatgaca gtgcctctgc caatatcacc atcgtggaat caaacgctac atctacatcc
accactggga caagccatct tgtaaaatgt gcggagaagg agaaaacttt ctgtgtgaat
ggaggggagt gcttcatggt gaaagacctt tcaaacccct cgagatactt gtgcaagtgc
ccaaatgagt ttactggtga tcgctgccaa aactacgtaa tggccagctt ctacagtacg
tccactccct ttctgtctct gcctgaatag

The amino acid sequence of full length human GGF2 is provided below:

(SEQ ID NO: 1)

MRWRRAPRRSGRPGPRAQRPGSAARSSPPLPLLPLLLLLGTAAL
APGAAAGNEAAPAGASVCYSSPPSVGSVQELAQRAAVVIEGKVHPQRR
QQGALDRKAAAAAGEAGAWGGDREPPAAGPRALGPPAEEPLLAANGTV
PSWPTAPVPSAGEPGEEAPYLVKVHQVWAVKAGGLKKDSLLTVRLGTW
GHPAFPSCGRLKEDSRYIFFMEPDANSTSRAPAAFRASFPPLETGRNLKK
EVSRVLCKRCALPPQLKEMKSQESAAGSKLVLRCETSSEYSSLRFKWFK
NGNELNRKNKPQNIKIQKKPGKSELRINKASLADSGEYMCKVISKLGND
SASANITIVESNATSTSTTGTSHLVKCAEKEKTFCVNGGECFMVKDLSN
PSRYLCKCPNEFTGDRCQNYVMASFYSTSTPFLSLPE

In a preferred embodiment, a functional fragment of GGF2 comprises a mature form of GGF2. For example, a mature form of GGF2 lacks an N-terminal signal sequence, e.g., the underlined sequence above. The amino acid sequence of a mature form of the human GGF2 peptide is provided below:

(SEQ ID NO: 2)

GNEAAPAGASVCYSSPPSVGSVQELAQRAAVVIEGKVHPQRRQQGALDRK
AAAAAGEAGAWGGDREPPAAGPRALGPPAEEPLLAANGTVPSWPTAPVPS
AGEPGEEAPYLVKVHQVWAVKAGGLKKDSLLTVRLGTWGHPAFPSCGRLK
EDSRYIFFMEPDANSTSRAPAAFRASFPPLETGRNLKKEVSRVLCKRCAL
PPQLKEMKSQESAAGSKLVLRCETSSEYSSLRFKWFKNGNELNRKNKPQN
IKIQKKPGKSELRINKASLADSGEYMCKVISKLGNDSASANITIVESNAT
STSTTGTSHLVKCAEKEKTFCVNGGECFMVKDLSNPSRYLCKCPNEFTG
DRCQNYVMASFYSTSTPFLSLPE

In other embodiments, a peptide of the invention is a variant of GGF2. For example, a variant of GGF2 comprises one of the amino acid sequences below:

(SEQ ID NO: 4)

VCLLTVAALPP,

(SEQ ID NO: 5)

ASPVSVGSVQELVQR,

(SEQ ID NO: 6)

WFVVIEGK,

(SEQ ID NO: 7)

KVHEVWAAK,

(SEQ ID NO: 8)

DLLLXV, wherein X = any amino acid,

(SEQ ID NO: 9)

LGAWGPPAFPVXY, wherein X = any amino acid,

(SEQ ID NO: 10)

YIFFMEPEAXSSG, wherein X = any amino acid,

(SEQ ID NO: 11)

KASLADSGEYMXK, wherein X = any amino acid.

In some embodiments, a peptide of the invention comprises a functional fragment of a variant of GGF2. A functional fragment of a variant of GGF2 binds to and activates an ErbB receptor and can have 422, 420, 418, 416, 414, 412, 410, 408, 406, 404, 402, 400, 398, 396, 394, 392, 390, 388, 386, 384, 382, 380, 379, 378, 377, 376, 375, 374, 373, 372, 371, 370, 369, 368, 367, 366, 365, 360, 355, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 55, 50, 45, 40, 35, 30, 25, 20 amino acids, or less of the full length GGF2 variant protein.

In some embodiments, an EGF-like domain-containing peptide of the invention comprises a fragment of a peptide encoded by an NRG-1, NRG-2, NRG-3, or NRG-4 gene, e.g., NRG-1 gene. For example, an EGF-like domain-containing peptide of the invention comprises one of the amino acid sequences below:

(SEQ ID NO: 12)

SHLVKCAEKEKTFCVNGGECFMVKDLSNPSRYLCKCPNEFTGDRCQNY
VMASFYKAEELYQ,

(SEQ ID NO: 13)

SHLVKCAEKEKTFCVNGGECFMVKDLSNPSRYLCKCPNEFTGDRCQNY
VMASFYKAEELY.

In other examples, an EGF-like domain-containing peptide of the invention comprises an EGFL domain 1 (EGFL1), EGFL domain 2 (EGFL2), EGFL domain 3 (EGFL3), EGFL domain 4 (EGFL4), EGFL domain 5 (EGFL5), or EGFL domain 6 (EGFL6). The amino acid sequences of EGFL1-EGFL6 and the nucleic acid, e.g., cDNA, sequence encoding these peptides are shown below.

EGFL1 amino acid sequence:

(SEQ ID NO: 14)

SHLVKCAEKEKTFCVNGGECFMVKDLSNPSRYLCKCPNEFTGDRCQNYV
MASFYSTSTPFLSLPE

EGFL1 is encoded by the following nucleic acid, e.g., cDNA, sequence:

(SEQ ID NO: 15)

agccatcttgtcaagtgtgcagagaaggagaaaactttctgtgtgaat
ggaggcgagtgcttcatggtgaaagacctttcaaatccctcaagatac
ttgtgcaagtgcccaaatgagtttactggtgatcgctgccaaaactacg
taatggccagcttctacagtacgtccactccctttctgtctctgcctga
atag

EGFL2 amino acid sequence:

(SEQ ID NO: 16)

SHLVKCAEKEKTFCVNGGECFMVKDLSNPSRYLCKCQPGFTGARCTENV
PMKVQTQEKAEELY

EGFL2 is encoded by the following nucleic acid, e.g., cDNA, sequence:

(SEQ ID NO: 17)

agccatcttgtcaagtgtgcagagaaggagaaaactttctgtgtgaatg
gaggcgagtgcttcatggtgaaagacctttcaaatccctcaagatactt
gtgcaagtgccaacctggattcactggagcgagatgtactgagaatgt
gcccatgaaagtccaaacccaagaaaaagcggaggagctctactaa

EGFL3 amino acid sequence:

(SEQ ID NO: 18)

SHLVKCAEKEKTFCVNGGECFMVKDLSNPSRYLCKCPNEFTGDRCQNYVM
ASFYKAEELY

EGFL3 is encoded by the following nucleic acid, e.g., cDNA, sequence:

(SEQ ID NO: 19)

agccatcttgtcaagtgtgcagagaaggagaaaactttctgtgtgaatgg
aggcgagtgcttcatggtgaaagacctttcaaatccctcaagatacttgt
gcaagtgcccaaatgagtttactggtgatcgctgccaaaactacgtaatg
gccagcttctacaaagcggaggagctctactaa

EGFL4 amino acid sequence:

(SEQ ID NO: 20)

SHLVKCAEKEKTFCVNGGECFMVKDLSNPSRYLCKCPNEFTGDRCQNYVM
ASFYKHLGIEFMEKAEELY

EGFL4 is encoded by the following nucleic acid, e.g., cDNA, sequence:

(SEQ ID NO: 21)

agccatcttgtcaagtgtgcagagaaggagaaaactttctgtgtgaatgg
aggcgagtgcttcatggtgaaagacctttcaaatccctcaagatacttgt
gcaagtgcccaaatgagtttactggtgatcgctgccaaaactacgtaatg
gccagcttctacaagcatcttgggattgaatttatggagaaagcggagga
gctctactaa

EGFL5 amino acid sequence:

(SEQ ID NO: 22)

SHLVKCAEKEKTFCVNGGECFMVKDLSNPSRYLCKCQPGFTGARCTENVP
MKVQTQEKCPNEFTGDRCQNYVMASFYSTSTPFLSLPE

EGFL5 is encoded by the following nucleic acid, e.g., cDNA, sequence:

(SEQ ID NO: 23)

agccatcttgtcaagtgtgcagagaaggagaaaactttctgtgtgaatgg
aggcgagtgcttcatggtgaaagacctttcaaatccctcaagatacttgt
gcaagtgccaacctggattcactggagcgagatgtactgagaatgtgccc
atgaaagtccaaacccaagaaaagtgcccaaatgagtttactggtgatcg
ctgccaaaactacgtaatggccagatctacagtacgtccactccctttct
gtctctgcctgaatag

EGFL6 amino acid sequence:

(SEQ ID NO: 24)

SHLVKCAEKEKTFCVNGGECFMVKDLSNPSRYLCKCQPGFTGARCTENVP
MKVQTQEKCPNEFTGDRCQNYVMASFYKAEELY

EGFL6 is encoded by the following nucleic acid, e.g., cDNA, sequence:

(SEQ ID NO: 25)

agccatcttgtcaagtgtgcagagaaggagaaaactttctgtgtgaatgg
aggcgagtgcttcatggtgaaagacctttcaaatccctcaagatacttgt
gcaagtgccaacctggattcactggagcgagatgtactgagaatgtgccc
atgaaagtccaaacccaagaaaagtgcccaaatgagtttactggtgatcg
ctgccaaaactacgtaatggccagcttctacaaagcggaggagctctact
aa

In some embodiments, a peptide of the invention is a purified recombinant or chemically synthesized peptide.

A peptide described herein, e.g., a peptide comprising an EGF-like domain, e.g., a neuregulin, such as a GGF2 or a functional fragment thereof, can be administered to patients, e.g., humans, veterinary subjects, or experimental animals with a pharmaceutically-acceptable diluent, carrier, or excipient. Compositions of the disclosure can be provided in unit dosage form. Therapeutic formulations can be in the form of liquid solutions or suspensions; for oral administration, formulations can be in the form of tablets or capsules; and for intranasal formulations, in the form of powders, nasal drops, or aerosols.

Methods for making formulations are found in, for example, “Remington's Pharmaceutical Sciences.” Formulations for parenteral administration can, for example, contain excipients, sterile water, or saline, polyalkylene glycols such as polyethylene glycol, oils of vegetable origin, or hydrogenated napthalenes. Other potentially useful parenteral delivery systems for administering molecules of the disclosure include ethylene-vinyl acetate copolymer particles, osmotic pumps, implantable infusion systems, and liposomes. Formulations for inhalation can contain excipients, for example, lactose, or may be aqueous solutions containing, for example, polyoxyethylene-9-lauryl ether, glycocholate and deoxycholate, or can be oily solutions for administration in the form of nasal drops, or as a gel.

The compositions, e.g., peptides, e.g., EGF-like domain containing peptides such as neuregulin, e.g., GGF2 or a fragment thereof, of the invention are provided for use as a pharmaceutical in the treatment, prevention, or delay of progression of a condition or disease described herein, e.g., heart failure. Also provided herein is the use of the present compositions, e.g., peptides, e.g., EGF-like domain containing peptides such as neuregulin, e.g., GGF2 or a fragment thereof, in the manufacture of a medicament for the treatment, prevention, or delay of progression of a condition disease described herein, e.g., heart failure.

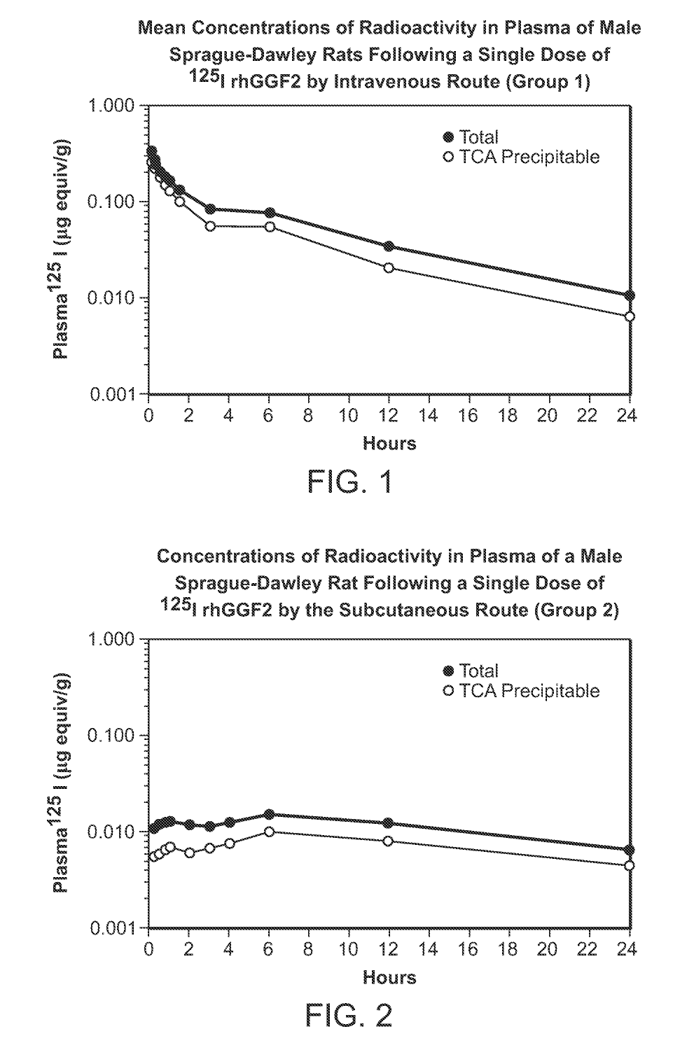

The half-life of neuregulin when delivered intravenously is 4 to 8 hours and when delivered subcutaneously is 11-15 hours. See, e.g., Tables 14 and 15 and FIGS. 1 and 2. Dosing at regimens as infrequent as every fourth day would, therefore, not maintain any detectable levels for at least three days between doses. Compounds with a half-life of this order are generally administered in accordance with a frequent dosing regimen, e.g., daily or multiple daily doses.

The present invention features a method that is based on the observation that therapeutic benefits of a peptide that comprises an epidermal growth factor-like (EGF-like) domain can be achieved by dosing regimens for administration of the peptide that do not maintain steady-state concentrations. The present inventors demonstrate herein that dosing regimens for neuregulin administration that do not maintain narrow steady-state concentrations are equally as effective as more frequent dosing regimens.

In accordance with the present disclosure, intermittent or discontinuous administration of a peptide described herein is directed to achieving a dosing regimen wherein narrow steady-state concentrations of the administered peptide are not maintained, thereby reducing the probability that the mammal will experience untoward side effects that may result from maintaining supraphysiological levels of the administered peptide over a prolonged duration. For example, side effects associated with supraphysiological levels of exogenously administered NRG include nerve sheath hyperplasia, mammary hyperplasia, renal nephropathy, hypospermia, hepatic enzyme elevation, heart valve changes and skin changes at the injection site.

In a preferred embodiment, the present disclosure is directed to an intermittent dosing regimen that elicits or permits fluctuations in the serum levels of the peptide comprising an EGF-like domain, e.g., a neuregulin, such as a GGF2 or a functional fragment thereof, and thus reduces the potential for adverse side effects associated with more frequent administration of the peptide. The intermittent dosing regimen of the present disclosure thus confers therapeutic advantage to the mammal, but does not maintain steady state therapeutic levels of the peptide. As appreciated by those of ordinary skill in the art, there are various embodiments of the disclosure to obtain the intermittent dosing; the benefits of these embodiments can be stated in various ways for example, the administering does not maintain steady state therapeutic levels of the peptide, the administering reduces potential for adverse side effects associated with administration of a NRG peptide more frequently, and/or the like.

In one aspect, the invention provides a method for treating heart failure in a mammal, the method comprising administering a peptide, e.g., exogenous peptide, comprising an epidermal growth factor-like (EGF-like) domain, e.g., a neuregulin, such as a GGF2 or a functional fragment thereof, to the mammal, wherein the administering at a dosing interval described herein reduces any potential adverse side effects that may be associated with administration of the peptide in the mammal. For example, a dosing interval is at least 48 hours, and administering at this interval does not maintain steady state levels of the peptide in the mammal and permits intradose fluctuation of serum concentrations of the peptide to baseline or pre-administration levels in the mammal.

Indeed, the present invention provides dosing intervals of a peptide described herein, e.g., a peptide comprising an EGF-like domain, e.g., a neuregulin, such as a GGF2 or a functional fragment thereof, of at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof so long as the interval/regimen is at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer. In certain embodiments, a peptide of the invention, e.g., a peptide comprising an EGF-like domain, e.g., a neuregulin, such as a GGF2 or a functional fragment thereof, is administered at dosing intervals of at least once per month, once per 2 months, once per 3 months, or once per 6 months. For example, the peptide is administered on a dosing interval for at least 2 weeks, e.g., at least 2 weeks, 3 weeks, or 4 weeks. For example, the peptide is administered on a dosing interval of greater than 4 months.

In some embodiments, a therapeutically effective amount of a peptide of the invention, e.g., a peptide comprising an EGF-like domain, e.g., a neuregulin, such as a GGF2 or a functional fragment thereof, is administered to a mammal at dosing intervals of 48, 72, 96 or more hours. Preferably, a dosing regimen comprises administering a therapeutically effective amount of the peptide to a mammal at dosing intervals of 72, 96 or more hours. Accordingly, the present method calls for intermittent or discontinuous administration (every 72 to 96 hours, or even longer intervals) of a peptide that contains an EGF-like domain, e.g., a neuregulin, such as a GGF2 or a functional fragment thereof, to the mammal, wherein administration of the peptide is in an amount effective to treat, prevent, or delay progression of heart failure in the mammal. Dosing regimens for neuregulin, e.g., GGF2 or a functional fragment thereof, administration that do not maintain steady-state concentrations are equally as effective as more frequent dosing regimens, yet without the inconvenience, costs or side effects that can result from more frequent administration.

As used herein the term intermittent or discontinuous administration includes a regimen for dosing on intervals of at least (or not less than) 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof so long as the interval/regimen is at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer. For example, the peptide is administered on a dosing interval for at least 2 weeks, e.g., at least 2 weeks, 3 weeks, or 4 weeks. For example, the dosing interval is greater than 4 months.

In certain embodiments, herein the term intermittent or discontinuous administration includes a regimen for dosing at least once every 2 weeks, once every 3 weeks, once every 4 weeks, once per month, once per 2 months, once per 3 months, once per 4 months, once per 5 months, once per 6 months, once per 7 months, once per 8 months, once per 9 months, once per 10 months, once per 11 months, or once per 12 months.

In certain embodiments of a dosing regimen of the disclosure, a peptide of the disclosure, e.g., a peptide comprising an EGF-like domain, e.g., a neuregulin, such as a GGF2 or a functional fragment thereof, is administered once every month, once every other month, once every three months, once every 3.5 months, once every 4 months, once every 4.5 months, once every 5 months, once every 6 months, once every 7 months, or on a less frequent dosing interval.

A dosing regimen of the disclosure can be initiated, established, or subsequently modified upon evaluation of a variety of factors, including, but not limited to ejection fraction (EF), left ventricular ejection fraction (LVEF), end-diastolic volume (EDV), end-systolic volume (ESV), heart volume, heart weight, liver toxicity, or increased or decreased protein expression levels in either cardiac tissue or blood samples of B-type Natiuretic Peptide (BNP), N-terminal B-type Natiuretic Peptide (NT BNP), and/or Troponin-I (TnI). A dosing regimen of the invention can also be initiated, established, or subsequently modified upon evaluation of, amelioration of, or improvement of one or more symptoms of heart failure, e.g., shortness of breath, exercise intolerance, hospitalization, re-hospitalization, mortality, and/or morbidity. A change in one or more of these factors may indicate that the interval between doses may be too small, the administration too frequent, or the route of administration not optimal. In other cases, a change in one or more of these factors may indicate that an optimal dose and/or dosing interval has been reached, and optionally, may be maintained.

In some cases liver toxicity is monitored, such as at regular intervals, e.g., liver toxicity is assessed at least every 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In some cases glucose levels, e.g., in plasma, serum, or blood of the subject, is monitored at regular intervals, e.g., liver toxicity is assessed at least every 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

For example, liver toxicity and/or glucose level is monitored on any dosing regimen described herein, e.g., on an escalating dosing regimen, a decreasing dosing regimen, and/or a dosing regimen in which a therapeutically effective dose is maintained and, e.g., not changed.

Conventional pharmaceutical practice is employed to provide suitable formulations or compositions, and to administer such compositions to patients or animals. Any appropriate route of administration may be employed, for example, parenteral, intravenous, subcutaneous, intramuscular, transdermal, intracardiac, intraperitoneal, intranasal, aerosol, oral, or topical, e.g., by applying an adhesive patch carrying a formulation capable of crossing the dermis and entering the bloodstream, administration. For example, the route of administration is intravenous or subcutaneous injection/infusion. For example, a peptide of the invention, e.g., an EGF-like domain-containing peptide, e.g., a neuregulin, such as GGF2 or a functional fragment thereof, is suitable for administration by a route described herein, e.g., intravenous or subcutaneous injection/infusion. In other examples, the compositions are delivered via a catheter, a pump delivery system, or a stent.

Dose levels of a peptide described herein, e.g., a peptide comprising an EGF-like domain, e.g., a neuregulin, such as a GGF2 or a functional fragment thereof, for example, administered via injection, such as intravenous or subcutaneous injection, range from about 0.001 mg/kg to about 4 mg/kg bodyweight. For example, the doses levels of the peptide range from about 0.001 mg/kg to about 1.5 mg/kg, from about 0.007 mg/kg to about 1.5 mg/kg, from about 0.001 mg/kg to about 0.02 mg/kg, from about 0.02 mg/kg to about 0.06 mg/kg, from about 0.06 mg/kg to about 0.1 mg/kg, from about 0.1 mg/kg to about 0.3 mg/kg, about 0.02 mg/kg to about 0.75 mg/kg, from about 0.3 mg/kg to about 0.5 mg/kg, from about 0.5 mg/kg to about 0.7 mg/kg, from about 0.5 mg/kg to about 1.0 mg/kg, from about 0.7 mg/kg to about 1.0 mg/kg, from about 0.3 mg/kg to about 4 mg/kg, from about 0.3 mg/kg to about 3.5 mg/kg, from about 1.0 mg/kg to about 1.5 mg/kg, or from about 1 mg/kg to about 10 mg/kg.

In some cases, the dose levels of the peptide are equal to or less than about 1.5 mg/kg bodyweight, e.g., equal to or less than about 0.8 mg/kg, or less than about 0.756 mg/kg bodyweight.

For example, the dose levels of the peptide include about 0.007 mg/kg, about 0.02 mg/kg, about 0.06 mg/kg, about 0.19 mg/kg, about 0.38 mg/kg, about 0.76 mg/kg, or about 1.5 mg/kg bodyweight, e.g., 0.007 mg/kg, 0.021 mg/kg, 0.063 mg/kg 0.189 mg/kg, 0.378 mg/kg, 0.756 mg/kg, or 1.512 mg/kg bodyweight.

In some examples, a peptide described herein, e.g., a peptide comprising an EGF-like domain, e.g., a neuregulin, such as a GGF2 or a functional fragment thereof, is administered at a dose level of about 0.005 mg/kg to about 4 mg/kg bodyweight on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In other examples, a peptide described herein, e.g., a peptide comprising an EGF-like domain, e.g., a neuregulin, such as a GGF2 or a functional fragment thereof, is administered at a dose level of about 0.007 mg/kg, about 0.02 mg/kg, about 0.06 mg/kg, about 0.19 mg/kg, about 0.38 mg/kg, about 0.76 mg/kg, or about 1.5 mg/kg bodyweight on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In some cases, a peptide described herein, e.g., a peptide comprising an EGF-like domain, e.g., a neuregulin, such as a GGF2 or a functional fragment thereof, is administered at a dose level of 0.007 mg/kg, 0.021 mg/kg, 0.063 mg/kg, 0.189 mg/kg, 0.378 mg/kg, 0.756 mg/kg, or 1.512 mg/kg bodyweight on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In other cases, a peptide described herein, e.g., a peptide comprising an EGF-like domain, e.g., a neuregulin, such as a GGF2 or a functional fragment thereof, is administered at a dose level of about 0.35 mg/kg to about 3.5 mg/kg bodyweight, e.g., about 3.5 mg/kg, about 1.75 mg/kg, about 0.875 mg/kg, or about 0.35 mg/kg bodyweight, on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

In some embodiments, the therapeutically effective amount of a peptide described herein, e.g., a neuregulin, such as GGF2 or a functional fragment thereof, is about 0.06 mg/kg bodyweight to about 0.38 mg/kg bodyweight and the dosing interval is at least 2 weeks, e.g., at least 2 weeks, 3 weeks, 4 weeks, 1 month, 2 months, 3 months (quarterly), 4 months, 5 months, 6 months, 7 months, 8 months, 9 months, 10 months, 11 months, 12 months, or longer. For example, the therapeutically effective amount of a peptide described herein is about 0.063 mg/kg, about 0.189 mg/kg, or about 0.375 mg/kg. For example, a therapeutically effective amount of the peptide of about 0.063 mg/kg, about 0.189 mg/kg, or about 0.375 mg/kg is administered via intravenous injection or infusion, e.g., to prevent, treat, or delay the progression of heart failure.

In some cases, a peptide described herein, e.g., a peptide comprising an EGF-like domain, e.g., a neuregulin, such as a GGF2 or a functional fragment thereof, is administered at a dose level of about 0.056 mg/kg to about 0.57 mg/kg bodyweight, e.g., about 0.056 mg/kg, about 0.1 mg/kg, about 0.2 mg/kg, about 0.3 mg/kg, about 0.4 mg/kg, or about 0.57 mg/kg, on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, or any combination or increment thereof.

The term, “about”, as used herein, refers to a stated value plus or minus another amount; thereby establishing a range of values. In certain preferred embodiments “about” indicates a range relative to a base (or core or reference) value or amount plus or minus up to 15%, 14%, 13%, 12%, 11%, 10%, 9%, 8%, 7%, 6%, 5%, 4%, 3%, 2%, 1%, 0.75%, 0.5%, 0.25% or 0.1%. For example, about refers to a range of +/−5% below and above the recited levels, e.g., dose levels.

The dose levels of the peptide described herein are administered via a route described above, e.g., intravenous or subcutaneous injection/infusion.

The dose level of a peptide of the disclosure, e.g., a peptide comprising an EGF-like domain, e.g., a neuregulin, such as a GGF2 or a functional fragment thereof, when administered by a subcutaneous route may be equal to or greater than the dose level of the same peptide when administered by an intravenous route. Moreover, the length of intervals between doses may decrease or the frequency of dosing may increase when the peptide, is administered by a subcutaneous route compared to an intravenous route. In certain embodiments, a subject who receives a peptide of the disclosure, by an intravenous route, and, subsequently demonstrates an increase of liver enzymes indicating liver toxicity, may be treated using an equivalent or greater dose of the peptide by a subcutaneous route.

Transdermal doses are generally selected to provide similar or lower blood levels than are achieved using injection doses.

In some dosing regimens of the invention, an initial dose of a peptide described herein, e.g., a peptide comprising an EGF-like domain, such as a neuregulin, e.g., GGF2 or a functional fragment thereof, is administered to the subject, and subsequent doses (e.g., a second dose, a third dose, a fourth dose, and so on) are administered to the subject on a dosing interval described herein. In some cases, the initial dose is the same as one or more of the subsequent doses. For example, the initial dose is the same as all subsequent doses. In some cases, the initial dose is lower than one or more of the subsequent doses, e.g., as provided by an escalating dosing regimen described herein. In other cases, the initial dose is higher than one or more of the subsequent doses, e.g., as provided by a decreasing dosing regimen described herein.

In some embodiments, the invention also provides a method for treating, preventing, or delaying the progression of heart failure in a subject in need thereof comprising administering to the subject a peptide described herein, e.g., a peptide comprising an EGF-like domain, e.g., a neuregulin, such as a GGF2 or a functional fragment thereof, according to an escalating dosing regimen. In some cases, the method includes administering a peptide described herein at a first therapeutically effective dose, and subsequently administering a second therapeutically effective dose. In some embodiments, the second dose is the same as the initial dose. In some embodiments, the second dose is higher than the first dose. In some cases, the method includes a step of administering one or more subsequent doses following the initial dose or the second dose, e.g., until a maintenance dose is reached. For example, the method includes administering the maintenance dose on a dosing interval described herein. For example, the dosing interval is at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months. For example, the dosing regimen comprises administering an initial dose of the peptide to the subject for a period of time, e.g., for at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, 2 years, 3 years, 4 years, 5 years, or longer, and subsequently increasing the dose at various designated time points, e.g., at time points of at least 24 h after each previous dose, such as time points of at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, 2 years, 3 years, 4 years, 5 years, or longer, after each previous dose.

For example, the dosing regimen comprises the steps of:
a) administering an initial dose of the peptide in the range of about 0.005 mg/kg bodyweight to about 1.5 mg/kg bodyweight, e.g., about 0.007 to about 0.015 mg/kg bodyweight, or about 0.007 mg/kg, about 0.021 mg/kg, about 0.063 mg/kg, about 0.189 mg/kg, about 0.378 mg/kg, about 0.756 mg/kg, or about 1.512 mg/kg bodyweight;
b) thereafter administering a second dose of the peptide that is 2-fold to 3-fold above the previous dose;
c) repeating step b) until a maintenance therapeutic dose is reached;
d) optionally, continuing to administer the maintenance therapeutic dose on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months or longer.

In some embodiments, the invention also provides a method for treating, preventing, or delaying the progression of heart failure in a subject in need thereof comprising administering to the subject a peptide described herein, e.g., a peptide comprising an EGF-like domain, e.g., a neuregulin, such as a GGF2 or a functional fragment thereof, according to decreasing dosing regimen. In some cases, the method includes administering a peptide described herein at a first therapeutically effective dose, and subsequently administering a second therapeutically effective dose. In some embodiments, the second dose is the same as the first dose. In some embodiments, the second dose is lower than the first dose. In some cases, the method includes a step of administering one or more subsequent doses following the initial dose or the second dose, e.g., until a maintenance dose is reached or until a dose of 0 mg/kg is reached. For example, the method includes administering the maintenance dose on a dosing interval described herein. For example, the dosing regimen comprises administering an initial dose of the peptide to the subject for a period of time, e.g., for at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, 2 years, 3 years, 4 years, 5 years, or longer, and subsequently decreasing the dose at various designated time points, e.g., at time points of at least 24 h after each previous dose, such as time points of at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, 2 years, 3 years, 4 years, 5 years, or longer, after each previous dose.

For example, the dosing regimen comprises the steps of:
e) administering an initial dose of the peptide in the range of about 0.005 mg/kg bodyweight to about 1.5 mg/kg bodyweight, e.g., about 0.007 to about 0.015 mg/kg bodyweight, or about 0.007 mg/kg, about 0.021 mg/kg, about 0.063 mg/kg, about 0.189 mg/kg, about 0.378 mg/kg, about 0.756 mg/kg, or about 1.512 mg/kg bodyweight;
f) thereafter administering a second dose of the peptide that is 2-fold to 3-fold below the previous dose;
g) repeating step b) until a maintenance therapeutic dose is reached;
h) optionally, continuing to administer the maintenance therapeutic dose on a dosing interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months or longer.

For example, the dosing regimen comprises the steps of:
i) administering an initial dose of the peptide in the range of about 0.005 mg/kg bodyweight to about 1.5 mg/kg bodyweight, e.g., about 0.007 to about 0.015 mg/kg bodyweight, or about 0.007 mg/kg, about 0.021 mg/kg, about 0.063 mg/kg, about 0.189 mg/kg, about 0.378 mg/kg, about 0.756 mg/kg, or about 1.512 mg/kg bodyweight;
j) thereafter administering a second dose of the peptide that is 2-fold to 3-fold above the previous dose; and
k) repeating step b) until a maximum therapeutic dose is reached.

The maximum therapeutic dose does not elicit an adverse event in the subject, and the doses are administered on an interval of at least 24 hours. For example, the maximum dose is about 0.7 mg/kg bodyweight to about 1.5 mg/kg bodyweight. For example, adverse events, such as treatment emergent adverse events (TEAEs), are shown in Table 12 and are graded using the Common Terminology Criteria for Adverse Events, version 4 (CTCAEv4).

In some cases, the method further comprises an additional step of continuing to administer the maximum therapeutically effective dose of the peptide at an interval of at least 24 hours. For example, the interval and/or period of time is at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, 2 years, 3 years, 4 years, 5 years, or longer). Alternatively or in addition, the method comprises an additional step of tapering or decreasing the dose, e.g., the initial dose or any subsequent dose, of the peptide over a period of time to a final dose of 0 mg/kg. For example, the period of time is over the course of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, 2 years, 3 years, 4 years, 5 years, or longer.

In some embodiments, a therapeutic dosing regimen, used in accordance with a method of the invention comprises the steps of
a) administering a therapeutically dose of the peptide in the range of about 0.005 mg/kg bodyweight to about 0.015 mg/kg bodyweight;
b) thereafter administering a therapeutically effective dose of the peptide wherein the doses are administered on an interval of at least 24 hours, e.g., at least 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer.

In some cases the therapeutic dose is a predetermined amount, wherein the predetermined amount is calculated by methods that are well known in the art.

In yet other cases the therapeutic dose is based on evaluating the efficacy of an initial dose, wherein efficacy is determined by methods that are well known in the art, e.g., as described herein.

Doses of a peptide described herein can be provided to the subject on a dosing interval described herein for as long as is required by the subject, e.g., for 1, 2, 3, 4, 5, 6, 7, 8, 9, 10, or more doses.

The basic principle of dosing is to determine an effective circulating concentration and design a dosing regimen to maintain those levels. Pharmacokinetic (PK) and pharmacodynamic (PD) studies are combined to predict a dosing regimen that will maintain a steady-state level of a particular drug. The typical plan is to minimize the difference between the Cmax and Cmin and thereby reduce side-effects. However, as described herein, in some embodiments, the present invention provides a dosing regimen of a peptide described herein that does not maintain a steady state level of the peptide, e.g., a discontinuous or intermittent dosing regimen, in a subject. For example, the dosing regimen minimizes exposure of the subject to the peptide while maintaining efficacy in treating, preventing, or delaying the progression of heart failure and/or one or more symptoms of heart failure.

Drugs are described by their ‘therapeutic index’ which is a ratio of the toxic dose or circulating levels divided by the effective dose or circulating concentrations. When the therapeutic index is large there is a wide safety range where an effective dose can be given without approaching toxic levels. When untoward effects result at concentrations too close to the effective concentrations the therapeutic index is described as narrow and the drug is difficult to administer safely.

While developing dosing regimens one combines the PK/PD data with knowledge of the therapeutic index to design a dose and frequency of administration such that the compound is maintained at a concentration in a patient, e.g., a human, such that it is above the effective concentration and below the toxic concentration. If an effective concentration of the drug cannot be maintained without inducing unsafe effects, the drug will fail during development. Additional commentary pertaining to drug development can be found in a variety of references, including: Pharmacokinetics in Drug Development: Clinical Study Design and Analysis (2004, Peter Bonate and Danny Howard, eds.), which is incorporated herein in its entirety.

Medical intervention involving drug treatment calls for the selection of an appropriate drug and its delivery at an adequate dosage regimen. An adequate dosage regimen involves a sufficient dose, route, frequency, and duration of treatment. The ultimate objective of drug therapy is the acquisition of optimal drug concentrations at the site of action so as to enable the treated patient to overcome the pathologic process for which treatment is necessitated. Broadly speaking, basic knowledge of the principles of drug disposition facilitates the selection of appropriate dosage regimens. Therapeutic drug monitoring (TDM) can, however, be used in this context as a supplemental tool to assist an attending physician in determining effective and safe dosage regimens of selected drugs for medical therapy of individual patients.

The definition of optimal drug concentration varies depending on the pharmacodynamic features of the particular drug. Optimal therapy for time-dependent antibiotics like penicillin, for example, is related to achieving peak concentration to MIC (minimum inhibitory concentration) ratios of 2-4 and a time above the MIC equal to 75% of the dose interval. For concentration-dependent antibiotics like gentamicin, for example, efficacy is related to obtaining peak concentration to MIC ratios of about 8-10. Irrespective of the nuances associated with administration of a particular drug, drug therapy aims to achieve target plasma concentrations (which often reflect the concentrations at the site of action) within the limits of a “therapeutic window”, which has been previously determined based on the pharmacokinetic, pharmacodynamic and toxicity profiles of the drug in the target species. The width of this window varies for different drugs and species. When the difference between the minimum efficacious concentration and the minimum toxic concentration is small (2 to 4-fold), the therapeutic window is referred to as narrow. In contrast, when there is a large difference between the effective and toxic concentration, the drug is viewed as having a wide therapeutic window. An example of a drug with a narrow therapeutic window is digoxin, in which the difference between the average effective and toxic concentrations is 2 or 3-fold. Amoxicillin, on the other hand, has a wide therapeutic range and overdosing of a patient is not generally associated with toxicity problems.

Pronounced variability among healthy subjects of the same species with respect drug responsiveness is common. Moreover, disease states have the potential to affect organ systems and functions, e.g., kidney, liver, water content, that may in turn affect drug responsiveness. This, in turn, contributes to increased differentials in drug responsiveness in sick individuals to whom the drug is administered. Yet another relevant issue relates to administration of more than one drug at a time, which results in pharmacokinetic interactions that can lead to alterations in responsiveness to one or both drugs. In summary, physiological, e.g., age, pathological, e.g., disease effects, and pharmacological, e.g., drug interaction, factors can alter the disposition of drugs in animals. Increased variability among individuals ensuing therefrom may result in therapeutic failure or toxicity in drugs with a narrow therapeutic window.

The proper timing of blood sampling for the purposes of determining serum drug level, as well as the interpretation of the reported level require consideration of the pharmacokinetic properties of the drug being measured. Some terms used in discussion of these properties are defined in the following paragraphs.

Half-life is the time required for the serum concentration present at the beginning of an interval to decrease by 50%. Knowing an approximate half-life is essential to the clinician since it determines the optimal dosing schedule, the intradose fluctuation of the serum concentration, and the time required to achieve steady state.

In brief, multiple pharmacokinetic studies have been performed for GGF2. Typical half-lives for GGF2 are between 4 and 8 hours for the intravenous (iv) route, whereas the half-life of subcutaneously (sc) administered GGF2 is between 11 and 15 hours. Cmax, AUC, Tmax and T1/2 are shown in Tables 14 and 15 below. Where the half-life was too long to be determined accurately by these methods, a dash is presented in lieu of a time.

TABLE 14
Mean Pharmacokinetics of ¹²⁵I-rhGGF2-Derived Radioactivity
in Plasma of Male Sprague-Dawley Rats Following a Single Intravenous
of Subcutaneous Dose of ¹²⁵I-rhGGF2.

| | |
|---|---|
| Group 1 (n = 2) | Group 2 (n = 1) |

| | | TCA | | TCA |
|---|---|---|---|---|
| Parameters | Total | Precip | Total | Precip |
| Cmax (μg eq/g) | 0.3289 | 0.2953 | 0.0157 | 0.01 |
| AUC ₀₋ₜ (μg eq/g) | 1.27 | 0.01 | 0.27 | 0.17 |
| AUG inf (μg eq/g) | 1.37 | 0.96 | 0.39 | 0.26 |
| Tmax (h) | 0.08 | 0.08 | 6.0 | 6.0 |
| Half-life | 6.37 | 6.11 | 13.20 | 14.66 |

| | |
|---|---|
| Group 1 - i.v. | Group 2 - s.c. |

TABLE 15
Mean Pharmacokinetics of ¹²⁵I-GGF2-Derived Radioactivity
in Plasma of Male Sprague-Dawley Rats Following a Side Intravenous
of Subcutaneous Dose of ¹²⁵I-rhGGF2.

| | |
|---|---|
| Group 1 (n = 2) | Group 2 (n = 1) |

| | | TCA | | TCA |
|---|---|---|---|---|
| Parameters | Total | Precip | Total | Precip |
| Cmax (μg eq/g) | 0.2611 | 0.2291 | 0.0197 | 0.0034 |
| AUC ₀₋ₜ (μg eq/g) | 1.488 | 0.567 | 0.335 | 0.064 |
| AUC inf (μg eq/g) | 1.667 | 0.62 | — | — |
| Tmax (h) | 0.08 | 0.08 | 12.0 | 12.0 |
| Half-life | 7.75 | 7.96 | — | — |

| | |
|---|---|
| Group 1 - i.v. | Group 2 - s.c. |

The plasma concentrations after administration are shown in FIGS. 1 and 2 for iv and sc administration, respectively. As shown in FIGS. 1 and 2, Cmax, refers to maximal plasma concentration (the maximum concentration that is measured in the plasma at any time after administration); AUCinf, refers to the area under the concentration versus time curve to time infinity (which method is used to anticipate that the assay has limits of detection); AUC₀₋ₜ, refers to the area under the plasma concentration (time curve from time zero to the last measurable concentration); AUC by any method refers to an estimate of the total exposure to the animal; and Tmax, refers to the median time of maximal plasma concentration.

As shown by the tables and figures provided, it is not possible to maintain steady state therapeutic levels by either dosing route with every fourth day, every other day or every day of dosing. Levels are unmeasurable after a day and even long before that, as reflected by the data set forth in Table 16.

TABLE 16
PK Parameters for GGF2 after Intravenous Administration*

| | | | | | | | |
|---|---|---|---|---|---|---|---|
| | | AUC_{0-∞}/ | | AUC₀₋ₗₐₛₜ/ | | | |
| | | Dose | | Dose | | | |
| Dose | AUC_{0-∞} | ((hr · ng/mL)/ | AUC₀₋ₗₐₛₜ | ((hr · ng/mL)/ | CL | | |
| (mg/kg) | (hr · ng/mL) | mg/kg) | (hr · ng/mL) | mg/kg) | (mL/min/kg) | T½ (h) | Vss (mL/kg) |

Rats

| | | | | | | | | | | | | | | |
|---|---|---|---|---|---|---|---|---|---|---|---|---|---|---|
| 8 | 16100 ± | 20500 | 2010 ± | 2560 | 16800 ± | 22300 | 2100 ± | 2790 | 18.1 ± | 12.7 | 1.46 ± | 1.84 | 1050 ± | 331 |
| 16 | 39600 ± | 9440 | 2470 ± | 590 | 38300 ± | 10000 | 2390 ± | 625 | 7.00 ± | 1.33 | 1.69 ± | 0.430 | 532 ± | 145 |

Monkeys

| | | | | | | | | | | | | | | |
|---|---|---|---|---|---|---|---|---|---|---|---|---|---|---|
| 8 | 15900 ± | 1690 | 1980 ± | 212 | 15100 ± | 1730 | 1890 ± | 217 | 8.48 ± | 0.91 0 | 2.02 ± | 0.358 | 1110 ± | 113 |

*taken from data obtained from plasma GGF2 concentrations measured by ELISA. Data reported are mean ± SD.

Steady state serum concentrations are those values that recur with each dose and represent a state of equilibrium between the amount of drug administered and the amount being eliminated in a given time interval. During long term dosage with any drug, the two major determinants of its mean steady state serum concentration are the rate at which the drug is administered and the drug's total clearance in that particular patient.

Peak serum concentration is the point of maximum concentration on the serum concentration-versus-time curve. The exact time of the peak serum concentration is difficult to predict since it represents complex relationships between input and output rates.

Trough serum concentration is the minimum serum concentration found during a dosing interval. Trough concentrations are theoretically present in the period immediately preceding administration of the next dose.

Absorption is the process by which a drug enters the body. Intravascularly administered drugs are absorbed totally, but extravascular administration yields varying degrees and rates of absorption. The relationship between the rate of absorption and the rate of elimination is the principle determinant of the drug concentration in the bloodstream.

Distribution is the dispersion of the systemically available drug from the intravascular space into extravascular fluids and tissues and thus to the target receptor sites.

Therapeutic range is that range of serum drug concentrations associated with a high degree of efficacy and a low risk of dose-related toxicity. The therapeutic range is a statistical concept: it is the concentration range associated with therapeutic response in the majority of patients. As a consequence, some patients exhibit a therapeutic response at serum levels below the lower limit of the range, while others require serum levels exceeding the upper limit for therapeutic benefit.

Correct timing of sample collection is important, since drug therapy is often revised on the basis of serum concentration determinations. The absorption and distribution phases should be complete and a steady-state concentration achieved before the sample is drawn. Levels obtained before a steady-state concentration exists may be erroneously low; increasing the dosage based on such a result could produce toxic concentrations. In addition, when making comparative measurements, it is important that the sampling time be consistent.

The timing of blood samples in relation to dosage is critical for correct interpretation of the serum concentration result. The selection of the time that the sample is drawn in relation to drug administration should be based on the pharmacokinetic properties of the drug, its dosage form and the clinical reason for assaying the sample, e.g., assessment of efficacy or clarification of possible drug-induced toxicity. For routine serum level monitoring of drugs with short half-lives, both a steady state peak and trough sample may be collected to characterize the serum concentration profile; for drugs with a long half-life, steady-state trough samples alone are generally sufficient.

In keeping with conventional wisdom and development practice, other medical treatments for CHF are typically administered on at least a daily basis. The periodicity of such a regimen is thought to be required because CHF is a chronic condition, commonly caused by impaired contraction and/or relaxation of the heart, rather than an acute condition. In persons with a weak or failing heart leading to impaired relaxation and CHF, medical treatments include drugs that block formation or action of specific neurohormones, e.g. angiotensin converting enzyme inhibitors (ACE-inhibitors), angiotensin receptor antagonists (ARBs), aldosterone antagonists and beta-adrenergic receptor blockers. These and other medications are now standard of care in chronic CHF as they have been demonstrated to result in improved symptoms, life expectancy and/or a reduction in hospitalizations. In the setting of acute exacerbation or chronic symptoms, patients are often treated with inotropes, e.g. dobutamine, digoxin, to enhance cardiac contractility, along with vasodilators, e.g. nitrates, nesiritide, and/or diuretics, e.g. furosemide, to reduce congestion. Patients with hypertension and congestive heart failure are treated with one or more antihypertensive agent such as beta-blockers, ACE-inhibitors and ARBs, nitrates, e.g., isosorbide dinitrate, hydralazine, and calcium channel blockers.

Thus, despite typical practice with respect to treatment of CHF, the present inventors have demonstrated that the dosing regimens described herein result in effective treatment of CHF, while avoiding undesirable side-effects. Although not wishing to be bound by theory, it is likely that such neuregulin treatment strengthens the pumping ability of the heart by stimulating cardiomyocyte hypertrophy, and partially or completely inhibits further deterioration of the heart by suppressing cardiomyocyte apoptosis.

Maintaining supranormal levels of exogenously supplied neuregulins has been shown to have untoward effects including nerve sheath hyperplasia, mammary hyperplasia, renal nephropathy, hypospermia, hepatic enzyme elevation, heart valve changes and skin changes at the injection site. These effects were observed following daily subcutaneous administration of neuregulin. See, e.g., Table 8. Developing dosing regimens to reduce these effects would significantly enhance the ability of neuregulins to be utilized as therapeutics and it is toward this end that the present disclosure is directed. To this end, the present invention demonstrates that less frequent dosing that does not maintain constant levels is also effective for use in treating heart failure.

The compounds of the disclosure, e.g., a peptide comprising an EGF-like domain, e.g., a neuregulin, such as a GGF2 or a functional fragment thereof, can be administered as the sole active agent or they can be administered in combination with other agents, including other compounds, e.g., peptides, that demonstrate the same or a similar therapeutic activity and that are determined to be safe and efficacious for such combined administration. Other such compounds used for the treatment of CHF include brain natriuretic peptide (BNP); statins (e.g., atorvastatin, fluvastatin, lovastatin, pitavastatin, pravastatin, rosuvastatin, or simvastatin); drugs that block formation or action of specific neurohormones (e.g. angiotensin converting enzyme inhibitors (ACE-inhibitors), angiotensin receptor antagonists (ARBs), aldosterone antagonists and beta-adrenergic receptor blockers); inotropes (e.g. dobutamine, digoxin) to enhance cardiac contractility; vasodilators (e.g. nitrates, nesiritide); diuretics (e.g. furosemide) to reduce congestion; one or more antihypertensive agents (such as beta-blockers, ACE-inhibitors and ARBs); nitrates (e.g., isosorbide dinitrate); hydralazine; and/or calcium channel blockers.

In particular embodiments of the compositions and methods of the disclosure, a benzodiazepine drug is administered to a patient within the same composition, or, alternatively, as part of the same treatment and/or in accordance with the same administration regimen as a peptide that comprises an epidermal growth factor-like (EGF-like) domain. Benzodiazepine drugs result from the fusion of a benzene ring and a diazepine ring. Benzodiazepine drugs may be classified as short-, intermediate-, or long-acting. Benzodiazepine drugs share anxiolytic, sedative, hypnotic, muscle relaxant, amnesic, anticonvulsant, and anti-hypertension properties. Exemplary benzodiazepine drugs of the disclosure include, but are not limited to, alprazolam, bretazenil, bromazepam, brotizolam, chlorodiazepoxide, cinolazepam, clobazam, clonazepam, clorazepate, clotiazepam, cloxazolam, delorazepam, diazepam, estazolam, eszopicloneetizolam, ethyl loflazepate, flumazenil, flunitrazepam, 5-(2-bromophenyl)-7-fluoro-1H-benzo[e][1,4]diazepin-2(3H)-one, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, purazolam, quazepam, temazepam, tetrazepam, triazolam, zaleplon, zolpidem, and zopiclone. The following exemplary benzodiazepine drugs may have anxiolytic properties: alprazolam, bretazenil, bromazepam, chlorodiazepoxide, clobazam, clonazepam, clorazepate, clotiazepam, cloxazolam, delorazepam, diazepam, etizolam, ethyl loflazepate, halazepam, ketazolam, lorazepam, medazepam, nordazepam, oxazepam, phenazepam, pinazepam, prazepam, premazepam, and purazolam. The following exemplary benzodiazepine drugs may have anticonvulsant properties: bretazenil, clonazepam, clorazepate, cloxazolam, diazepam, flutoprazepam, lorazepam, midazolam, nitrazepam, and phenazepam. The following exemplary benzodiazepine drugs may have hypnotic properties: brotizolam, estazolam, eszopiclone, flunitrazepam, flurazepam, flutoprazepam, loprazolam, lormetazepam, midazolam, nimetazepam, nitrazepam, quazepam, temazepam, triazolam, zaleplon, zolpidem, and zopiclone. The following exemplary benzodiazepine drug may have sedative properties: cinolazepam. The following exemplary benzodiazepine drugs may have muscle relaxant properties: diazepam and tetrazepam.

In particular embodiments of the compositions and methods of the disclosure, midazolam is administered to a patient within the same composition, or, alternatively, as part of the same treatment and/or in accordance with the same administration regimen as a peptide that comprises an epidermal growth factor-like (EGF-like) domain, e.g., a neuregulin, such as a GGF2 or a functional fragment thereof. In certain aspects of these embodiments, midazolam is administered to a patient within the same composition, or, alternatively, as part of the same treatment and/or in accordance with the same administration regimen as a peptide that comprises an epidermal growth factor-like (EGF-like) domain, e.g., a neuregulin, such as a GGF2 or a functional fragment thereof. The neuregulin may be neuregulin 1 (NRG1). The neuregulin may be GGF2 or a functional fragment thereof. Although a benzodiazepine drug, e.g. midazolam, may be administered according to any dosing regimen described in the disclosure, in particular embodiments, the benzodiazepine drug, e.g. midazolam, may be administered in one or more doses, including oral doses. In certain aspects, when the benzodiazepine drug, e.g. midazolam, is administered in one or more doses, including oral doses, the peptide, e.g., peptide comprising an EGF-like domain, e.g., a neuregulin, such as a GGF2 or a functional fragment thereof, is administered in a single dose, e.g. a single intravenous infusion. The benzodiazepine drug, e.g. midazolam, may be administered prior to, simultaneously with, or following a dose of the neuregulin, e.g. GGF2 or functional fragment thereof. In a particular aspect of this embodiment, a benzodiazepine drug, e.g. midazolam, is administered in 5 oral doses, after the second of which, a neuregulin, e.g. GGF2 or functional fragment thereof, is administered in a single dose, e.g. a single intravenous infusion.

Midazolam is a short-acting benzodiazepine drug and central nervous system (CNS) depressant. Midazolam is approved for the treatment of seizures, insomnia, sedation and/or amnesia before medical/surgical procedures, and induction or maintenance of anesthesia. Midazolam possesses potent anxiolytic, amnestic, hypnotic, anticonvulsant, muscle relaxant, and sedative properties. Midazolam enhances the effect of the neurotransmitter GABA on the GABA_{A} receptors, causing an increased frequency of chlorine channel opening, and, therefore, inducing or increasing inhibition of neural activity.

Midazolam may be administered by any route, including, but not limited to, intranasal and oral, e.g. buccal route of absorption via the gums and cheek. Midazolam has an elimination half-life of approximately one to four hours. The elimination half-life may be extended in young children, adolescents, and the elderly.

Subjects who receive a composition of the disclosure or subject treated in accordance with a method of the disclosure may take one or more benzodiazepine drugs prior to administration of a composition or initiation of a treatment regimen of the disclosure. Subjects who receive a composition of the disclosure or subject treated in accordance with a method of the disclosure may take one or more benzodiazepine drugs during administration of a composition or initiation of a treatment regimen of the disclosure. Subjects who receive a composition of the disclosure or subject treated in accordance with a method of the disclosure may take one or more benzodiazepine drugs following administration of a composition or initiation of a treatment regimen of the disclosure.

Suitable subjects or patients include mammals. Mammals include, but are not limited to, humans, mice, rats, rabbits, dogs, monkeys or pigs. In one embodiment of the disclosure, the mammal is a human. Subjects of the treatment methods provided in this disclosure may present with chronic heart failure. Preferably, the subject's condition has remained stable for at least 1, 2, 3, 4, 5, or 6 months. Stable or chronic heart failure may be further characterized by the lack of increase or decrease in heart function and/or damage over a period of at least 1, 2, 3, 4, 5, or 6 months. For example, the subject has suffered from chronic heart failure for at least 1 month, e.g., at least 1, 2, 3, 4, 5, 6, or more months, prior to administration of a peptide of the invention.

For example, the subject suffers from class 2, 3, or 4 heart failure prior to administration of a peptide of the invention. The New York Heart Association (NYHA) Functional Classification system is used to determine the class of heart failure based on based on how much the subject is limited during physical activity. Patients who fall under class 1 heart failure have cardiac disease but no limitation of physical activity. Ordinary physical activity does not cause excessive fatigue, palpitation, dyspnea or anginal pain. Patients who fall under class 2 heart failure have cardiac disease that results in slight limitation of physical activity. These patients are comfortable at rest, but ordinary physical activity causes fatigue, palpitation, dyspnea or anginal pain. Class 3 heart failure patients have cardiac disease that results in significant limitation of physical activity. Although these patients are comfortable at rest, less than ordinary physical activity results in fatigue, palpitation, dyspnea or anginal pain. Class IV heart failure patients have cardiac disease that results in an inability to perform any physical activity without discomfort. At rest, these patients may experience symptoms of heart failure or anginal syndrome. Any physical activity increases the discomfort level.

In some cases, the subject suffers from systolic heart failure. For example, the subject suffers from systolic left ventricular dysfunction. For example, the subject has a left ventricular ejection fraction of 40% or less, e.g., 40%, 35%, 30%, 25%, 20%, 15%, 10%, or less, prior to administration of peptide described herein.

In some examples, the subject is a human of at least 18 years of age, e.g., at least 18, 20, 25, 30, 35, 40, 45, 50, 55, 60, 65, 70, 75, 80, 85, 90, or 95. In some cases, the human is between 18-75 years of age.

In some cases, the subject may suffer from acute decompensated heart failure (ADHD) prior to administration of a peptide described herein. For example, acute decompensated heart failure is characterized by a sudden or gradual onset of one or more symptoms or signs of heart failure that requires emergency room visits, hospitalization, and/or unplanned doctor office visits. In some cases, ADHD is associated with pulmonary and/or systemic congestion, which may be caused by an increase in left and/or right heart filling pressures. See, e.g., Joseph et al. Tex. Heart Inst. J. 36.6(2009):510-20. For example, ADHD can be diagnosed by measuring the level of plasma B-type natriuretic peptide (BNP) or N-terminal pro-B-type natriuretic peptide (NT-proBNP) in a subject, using methods commonly known in the art. For example, a BNP level in a biological sample (such as blood, plasma, serum, or urine) from a subject that is higher than 100 pg/dL, e.g., at least 100 pg/dL, 200 pg/dL, 300 pg/dL, 400 pg/dL, 500 pg/dL, 600 pg/dL or higher, may indicate that a subject has ADHD. In some examples, a therapeutic dosing regimen of a peptide described herein is sufficient to prevent, reduce, or delay the occurrence of ADHD.

In some embodiments, the heart failure may result from hypertension, ischemic heart disease, exposure to a cardiotoxic compound, e.g., cocaine, alcohol, an anti-ErbB2 antibody or anti-HER antibody, such as HERCEPTIN®, or an anthracycline antibiotic, such as doxorubicin or daunomycin, myocarditis, thyroid disease, viral infection, gingivitis, drug abuse, alcohol abuse, periocarditis, atherosclerosis, vascular disease, hypertrophic cardiomyopathy, acute myocardial infarction or previous myocardial infarction, left ventricular systolic dysfunction, coronary bypass surgery, starvation, radiation exposure, an eating disorder, or a genetic defect.

In another embodiment of the disclosure, an anti-ErbB2 or anti-HER2 antibody, such as HERCEPTIN®, is administered to the mammal before, during, or after anthracycline administration.

In other embodiments of the disclosure, a peptide, e.g., a peptide comprising an EGF-like domain, e.g., a neuregulin, such as a GGF2 or a functional fragment thereof, is administered prior to exposure to a cardiotoxic compound, during exposure to the cardiotoxic compound, or after exposure to the cardiotoxic compound; the peptide is administered prior to or after the diagnosis of congestive heart failure in the mammal. A method of the disclosure can take place after the subject mammal has undergone compensatory cardiac hypertrophy. In some examples, an outcome of a method described herein is to maintain left ventricular hypertrophy, to prevent/delay progression of myocardial thinning, or to inhibit cardiomyocyte apoptosis. In a method of the disclosure, the peptide can comprise, consist essentially of, or consist of an EGF-like domain, e.g., a neuregulin, such as a GGF2 or a functional fragment thereof. The peptide is administered before, during, or after exposure to a cardiotoxic compound. In another embodiment, the peptide is administered during two, or all three, of these periods. In other embodiments of the disclosure, the peptide is administered either prior to or after the diagnosis of congestive heart failure in the mammal. In yet another embodiment of the disclosure, the peptide is administered to a mammal that has undergone compensatory cardiac hypertrophy. In other particular embodiments of the disclosure, administration of the peptide maintains left ventricular hypertrophy, prevents/delays progression of myocardial thinning, and/or inhibits cardiomyocyte apoptosis.

In other embodiments, a subject in need of a treatment or prophylaxis described herein is at risk for heart failure, e.g., congestive heart failure. Risk factors that increase the likelihood of an individual's developing congestive heart failure are well known. These include, and are not limited to, smoking, obesity, high blood pressure, ischemic heart disease, vascular disease, coronary bypass surgery, myocardial infarction, left ventricular systolic dysfunction, exposure to cardiotoxic compounds (alcohol, drugs such as cocaine, and anthracycline antibiotics such as doxorubicin, and daunorubicin), viral infection, pericarditis, myocarditis, gingivitis, thyroid disease, radiation exposure, genetic defects known to increase the risk of heart failure (such as those described in Bachinski and Roberts, Cardiol. Clin. 16:603-610, 1998; Siu et al., Circulation 8:1022-1026, 1999; and Arbustini et al., Heart 80:548-558, 1998), starvation, eating disorders such as anorexia and bulimia, family history of heart failure, and myocardial hypertrophy.

In some embodiments, the patient population that would benefit from a treatment regimen of the present disclosure is quite diverse, e.g., patients with impaired kidney function are good candidates because continuous levels of protein therapeutics are often associated with renal glomerular deposits. The utility of a therapeutic regimen that does not maintain constant plasma levels as is described in this disclosure would, therefore, be very beneficial for patients with compromised renal function in which any diminution of existing function could be deleterious. Similarly, brief and intermittent exposure to a therapeutic such as GGF2 or a functional fragment, as described herein, can be beneficial for patients with tumor types that are responsive to chronic and continuous stimulation with a growth factor. Other patients that may specifically benefit from intermittent therapy as described herein are patients with schwannomas and other peripheral neuropathies. It is an advantage of the present disclosure that intermittent dosing may have significant advantages in not maintaining continuous side-effect-related stimulation of various tissues.

In accordance with the present disclosure, a peptide described herein, e.g., a peptide comprising an EGF-like domain, e.g., a neuregulin, such as a GGF2 or a functional fragment thereof, can be administered intermittently to achieve prophylaxis such as by preventing or delaying/decreasing the rate of congestive heart disease progression in those identified as being at risk. For example, administration of the peptide to a patient in early compensatory hypertrophy permits maintenance of the hypertrophic state and prevents/delays the progression to heart failure. In addition, those identified to be at risk may be given cardioprotective treatment with the peptide prior to the development of compensatory hypertrophy.

Administration of a peptide described herein, e.g., a peptide comprising an EGF-like domain, e.g., a neuregulin, such as a GGF2 or a functional fragment thereof, to cancer patients prior to and during anthracycline chemotherapy or anthracycline/anti-ErbB2 (anti-HER2) antibody, e.g., HERCEPTIN®, combination therapy can prevent/delay a patient's cardiomyocytes from undergoing apoptosis, thereby preserving cardiac function. Patients who have already suffered cardiomyocyte loss also derive benefit from neuregulin treatment, because the remaining myocardial tissue responds to neuregulin exposure by displaying hypertrophic growth and increased contractility.

In accordance with a method of the invention, administration of a peptide described herein, e.g., a peptide comprising an EGF-like domain, such as a neuregulin, e.g., GGF2 or a functional fragment thereof, e.g., at a therapeutically effective dose, is sufficient to ameliorate or stabilize a symptom of heart failure in a subject. Symptoms include but are not limited fatigue, shortness of breath, exercise intolerance, hospitalization, re-hospitalization, mortality, and/or morbidity. In some embodiments, administration or use of a peptide described herein, e.g., a peptide comprising an EGF-like domain, e.g., a neuregulin, such as a GGF2 or a functional fragment thereof, causes an improvement in and/or stabilization of one or more metrics of heart function. For example, administration or use of a therapeutically effective dose of a peptide described herein is sufficient to improve one or more metrics of heart function. In other embodiments, a therapeutically effective dose of a peptide described herein in sufficient to maintain and/or stabilize one or more metrics of heart function, or one or more symptoms of heart failure as described above. For example, a therapeutically effective dose of a peptide described herein is sufficient to maintain and/or stabilize one or more metrics of heart function or one or more symptoms of heart failure for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

Exemplary metrics of heart function include but are not limited to ventricular ejection fraction (EF), e.g., left ventricular ejection fraction (LVEF), end systolic volume (ESV), end diastolic volume (EDV), fractional shortening (FS), number of hospitalizations, exercise tolerance, mitral valve regurgitation, dyspnea, peripheral edema, and occurrence of ADHD. An improvement in heart function, e.g., as a result of administration of a peptide of the invention, is detected, e.g., by one or more of the following: an increase in LVEF, a decrease in ESV, a decrease in EDV, an increase in FS, a decrease in the number of hospitalizations, an increase in exercise tolerance, a decrease in the number of occurrences in or the severity of mitral valve regurgitation, a decrease in dyspnea, a decrease in peripheral edema, and prevention or reduction in occurrence of ADHD. In some examples, where a subject suffers from heart failure with preserved LVEF, a metric of heart function includes but is not limited to ESV, EDV, FS, number of hospitalizations, exercise tolerance, mitral valve regurgitation, dyspnea, occurrence of ADHD, and peripheral edema.

In some examples, administration of a therapeutically effective amount of a peptide described herein, e.g., a peptide comprising an EGF-like domain, e.g., a neuregulin, such as a GGF2 or a functional fragment thereof, is sufficient to increase the LVEF in the subject by at least 1%, e.g., at least 1, 2, 3, 4, 5, 6, 7, 8, 9, 10, 15, 20, 25, 30% or greater, compared to the LVEF prior to administration of the peptide. For example, the increase in LVEF is at least 1-20%. In some cases a therapeutically effective amount of a peptide described herein is sufficient to increase the LVEF of the subject in need thereof to an ejection fraction of about 10-40%, e.g., the LVEF of the subject is increased to an ejection fraction of about 10%, 15%, 20%, 25%, 30%, 35%, or about 40%. In other cases, a therapeutically effective amount of a peptide described herein is sufficient to increase the LVEF of the subject in need thereof to an ejection fraction of about 40-60%, e.g., the LVEF of the subject is increased to an ejection fraction of about 40%, 45%, 50%, 55%, or about 60%. In yet other cases a therapeutically effective amount of a peptide described herein is sufficient completely restore the LVEF of the subject in need thereof to a normal LVEF value. For example, the LVEF of the subject increases within 90 days or less, e.g., within 90 d, 80 d, 70 d, 60 d, 50 d, 40 d, 30 d, 20 d, 10 d or less, of the first administration, e.g., initial dose, of the peptide in the subject. In some cases, the increased LVEF in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide. For example, a therapeutically effective dose of a peptide described herein is sufficient to maintain and/or stabilize the LVEF in the subject for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

In some examples, administration of a therapeutically effective amount of a peptide described herein, e.g., a peptide comprising an EGF-like domain, e.g., a neuregulin, such as a GGF2 or a functional fragment thereof, is sufficient to decrease the EDV in the subject by at least 1 mL, e.g., at least 1 mL, 5 mL, 10 mL, 15 mL, 20 mL, 25 mL, 30 mL, 40 mL, 50 mL, 60 mL, 70 mL, 80 mL, 90 mL, 100 mL, or greater, e.g., at least 1-60 mL, compared to the EDV of the subject prior to administration of the peptide. For example, the EDV of the subject decreases within 90 days or less, e.g., within 90 d, 80 d, 70 d, 60 d, 50 d, 40 d, 30 d, 20 d, 10 d or less, of the first administration of the peptide in the subject, e.g., the initial dose of the peptide. In some cases, the decreased EDV in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

In other examples, administration of a therapeutically effective amount of a peptide described herein, e.g., a peptide comprising an EGF-like domain, e.g., a neuregulin, such as a GGF2 or a functional fragment thereof, is sufficient to decrease the ESV in the subject by at least 1 mL, e.g., at least 1 mL, 5 mL, 15 mL, 20 mL, 25 mL, 30 mL, 40 mL, 50 mL, 60 mL, 70 mL, 80 mL, 90 mL, 100 mL, or greater, e.g., at least 1-30 mL, compared to the ESV of the subject prior to administration of the peptide. For example, the ESV of the subject decreases within 90 days or less, e.g., within 90 d, 80 d, 70 d, 60 d, 50 d, 40 d, 30 d, 20 d, 10 d or less, of the first administration of the peptide in the subject, e.g., the initial dose of the peptide. In some cases, the decreased ESV in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

In some cases, administration of a therapeutically effective amount of a peptide described herein, e.g., a peptide comprising an EGF-like domain, e.g., a neuregulin, such as a GGF2 or a functional fragment thereof, is sufficient to increase the FS in the subject by at least 1%, e.g., at least 1, 2, 3, 4, 5, 6, 7, 8, 9, 10, 15, 20, 25, 30% or greater, compared to the FS prior to administration of the peptide. For example, the increase in FS is at least 1-15%. In some cases a therapeutically effective amount of a peptide described herein is sufficient to increase the FS of the subject in need thereof to a Percent Fractional Shortening of about 15%, e.g. about 1%, 2%, 3%, 4%, 6%, 7%, 8%, 9%, 10%, or about 15%. In other cases a therapeutically effective amount of a peptide described herein is sufficient to increase the FS of the subject in need thereof to a Percent Fractional Shortening of about 15-20%, e.g., about 15%, 16%, 17%, 18%, 19%, or about 20%. In yet other cases a therapeutically effective amount of a peptide described herein is sufficient to increase the FS of the subject in need thereof to a Percent Fractional Shortening of about 20-25%, e.g., about 20%, 21%, 22%, 23%, 24%, or about 25%. In further cases, a therapeutically effective amount of a peptide described herein is sufficient to increase the FS of the subject in need thereof to a Percent Fractional Shortening of about 25-45%, e.g., about 25%, 26%, 27%, 28%, 29%, 30%, 31%, 32%, 33%, 34%, 35%, 36%, 37%, 38%, 39%, 40%, 41%, 42%, 43%, 44%, or about 45%. For example, the FS of the subject increases within 90 days or less, e.g., within 90 d, 80 d, 70 d, 60 d, 50 d, 40 d, 30 d, 20 d, 10 d or less, of the first administration of the peptide in the subject, e.g., the initial dose of the peptide. In some cases, the increased FS in the subject is maintained for at least 12 hours, e.g., at least 12 hours, 24 hours, 36 hours, 48 hours, 72 hours, 96 hours, 1 day, 2 days, 3 days, 4 days, 5 days, 6 days, 7 days, 8 days, 9 days, 10 days, 11 days, 12 days, 13 days, 14 days, 90 days, 1 week, 2 weeks, 3 weeks, 4 weeks, 1 month, 2 months, 3 months (quarterly), 4 months, 5 months, 6 months, 7 months, 8 months, 9 months, 10 months, 11 months, 12 months, or longer, following the first administration of the peptide, e.g., without a subsequent administration of the peptide.

The metrics for assessing heart function described herein are determined by methods commonly known in the art.

The term “a” entity or “an” entity refers to one or more of that entity. For example, reference to “a peptide” includes a mixture of two or more such peptides, and the like. As such, the terms “a”, “an”, “one or more” and “at least one” can be used interchangeably. For example, “a dose” includes one or more doses. Further, unless otherwise required by context, singular terms shall include pluralities and plural terms shall include the singular.

As used herein, the term about is a stated value plus or minus another amount; thereby establishing a range of values. In certain preferred embodiments “about” indicates a range relative to a base (or core or reference) value or amount plus or minus up to 15%, 14%, 13%, 12%, 11%, 10%, 9%, 8%, 7%, 6%, 5%, 4%, 3%, 2%, 1%, 0.75%, 0.5%, 0.25% or 0.1%.

As used herein, the term adverse or deleterious side effect refers to an unintended and undesirable consequence of a medical treatment. With respect to the present disclosure, an adverse or deleterious side effect resulting from administration of a peptide, e.g., exogenous peptide, may include any one or more of the following: nerve sheath hyperplasia, mammary hyperplasia, renal nephropathy, and skin changes at the injection site, and/or an adverse event listed in Table 12.

Polynucleotides, peptides (which can also be referred to as polypeptides), or other agents described herein are, e.g., purified and/or isolated. Specifically, as used herein, an “isolated” or “purified” nucleic acid molecule, polynucleotide, peptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated peptide is free of the amino acids or sequences that flank it in its naturally-occurring state. Purified also defines a degree of sterility that is safe for administration to a human subject, e.g., lacking infectious or toxic agents.

As used herein, exogenous refers to a composition, e.g., a peptide, that is introduced from or produced outside a subject in need of a treatment described herein.

As used herein, cDNA (complementary DNA) is DNA that is synthesized, e.g., chemically synthesized, from a messenger RNA (mRNA) template. For example, the cDNA is synthesized from the mRNA template in a reaction catalyzed by enzymes such as reverse transcriptase and DNA polymerase.

As used herein, intradose fluctuation of serum concentrations of a peptide to pre-administration levels in a mammal refers to the difference between serum concentration levels before administration of a dose of the peptide.

As used herein, the term “steady state levels” refers to a level(s) of an exogenous agent, e.g., a peptide, that is sufficient to achieve equilibration (within a range of fluctuation between succeeding doses) between administration and elimination. “Maintaining steady state therapeutic levels” refers to sustaining the concentration of an exogenous agent at a level sufficient to confer therapeutic benefit to a subject or patient.

By “congestive heart failure” is meant impaired cardiac function that renders the heart unable to maintain the normal blood output at rest or with exercise, or to maintain a normal cardiac output in the setting of normal cardiac filling pressure. A left ventricular ejection fraction of about 40% or less is indicative of congestive heart failure (by way of comparison, an ejection fraction of about 60% percent is normal). Patients in congestive heart failure display well-known clinical symptoms and signs, such as tachypnea, pleural effusions, fatigue at rest or with exercise, contractile dysfunction, and edema. Congestive heart failure is readily diagnosed by well-known methods (see, e.g., “Consensus recommendations for the management of chronic heart failure.” Am. J. Cardiol., 83(2A):1A-38-A, 1999, incorporated herein by reference).

Relative severity and disease progression are assessed using well known methods, such as physical examination, echocardiography, radionuclide imaging, invasive hemodynamic monitoring, magnetic resonance angiography, and exercise treadmill testing coupled with oxygen uptake studies.

By “ischemic heart disease” is meant any disorder resulting from an imbalance between the myocardial need for oxygen and the adequacy of the oxygen supply. Most cases of ischemic heart disease result from narrowing of the coronary arteries, as occurs in atherosclerosis or other vascular disorders.

By “myocardial infarction” is meant a process by which ischemic disease results in a region of the myocardium being replaced by scar tissue.

By “cardiotoxic” is meant a compound that decreases heart function by directly or indirectly impairing or killing cardiomyocytes.

By “hypertension” is meant blood pressure that is considered by a medical professional, e.g., a physician or a nurse, to be higher than normal and to carry an increased risk for developing congestive heart failure.

By “treating” is meant that administration of a peptide described herein, e.g., a peptide comprising an EGF-like domain, e.g., a neuregulin or neuregulin-like peptide, a GGF2, or a functional fragment thereof, slows or inhibits the progression of heart failure, e.g., congestive heart failure, during the treatment, relative to the disease progression that would occur in the absence of treatment, in a statistically significant manner. Well known indicia such as left ventricular ejection fraction, exercise performance, mitral valve regurgitation, dyspnea, peripheral edema, and other clinical tests as enumerated above, as well as survival rates and hospitalization rates may be used to assess disease progression. Whether or not a treatment slows or inhibits disease progression in a statistically significant manner may be determined by methods that are well known in the art (see, e.g., SOLVD Investigators, N. Engl. J. Med. 327:685-691, 1992 and Cohn et al., N. Engl. J Med. 339:1810-1816, 1998, incorporated herein by reference).

By “preventing” is meant minimizing or partially or completely inhibiting the development of heart failure, e.g., congestive heart failure, in a subject at risk for developing heart failure, e.g., congestive heart failure (as defined in “Consensus recommendations for the management of chronic heart failure.” Am. J. Cardiol., 83(2A):1A-38-A, 1999, incorporated herein by reference). Determination of whether heart failure, e.g., congestive heart failure, is minimized or prevented by administration of a peptide of the invention is made by known methods, such as those described in SOLVD Investigators, supra, and Cohn et al., supra.

The term “therapeutically effective amount” is intended to mean that amount of a drug or pharmaceutical agent, e.g., a peptide described herein, that elicits the biological or medical response of a tissue, a system, animal or human that is being sought by a researcher, veterinarian, medical doctor or other clinician. A therapeutic change is a change in a measured biochemical characteristic in a direction expected to alleviate the disease or condition being addressed. More particularly, a “therapeutically effective amount” is an amount sufficient to decrease the symptoms associated with a medical condition or infirmity, to normalize body functions in disease or disorders that result in impairment of specific bodily functions, or to provide improvement in one or more of the clinically measured parameters of a disease.

The term “prophylactically effective amount” is intended to mean that amount of a pharmaceutical drug, e.g., a peptide described herein, that will prevent, reduce the risk of occurrence, or delay the progression of the biological or medical event that is sought to be prevented/delayed in a tissue, a system, animal or human by a researcher, veterinarian, medical doctor or other clinician.

The term “therapeutic window” is intended to mean the range of dose between the minimal amount to achieve any therapeutic change, and the maximum amount which results in a response that is the response immediately before toxicity to the subject.

By “at risk for heart failure”, e.g., at risk for congestive heart failure, is meant, e.g., an individual who smokes, is obese, i.e., 20% or more over their ideal weight, has been or will be exposed to a cardiotoxic compound (such as an anthracycline antibiotic), or has (or had) high blood pressure, ischemic heart disease, a myocardial infarct, a genetic defect known to increase the risk of heart failure, a family history of heart failure, myocardial hypertrophy, hypertrophic cardiomyopathy, left ventricular systolic dysfunction, coronary bypass surgery, vascular disease, atherosclerosis, alcoholism, periocarditis, a viral infection, gingivitis, or an eating disorder, e.g., anorexia nervosa or bulimia, or is an alcoholic or cocaine addict.

By “decreasing progression of myocardial thinning” is meant maintaining hypertrophy of ventricular cardiomyocytes such that the thickness of the ventricular wall is maintained or increased.

By “inhibits myocardial apoptosis” is meant that administration of a peptide described herein, e.g., a peptide comprising an EGF-like domain, e.g., a neuregulin, such as a GGF2 or a functional fragment thereof, inhibits death of cardiomyocytes by at least 10%, more preferably by at least 15%, still more preferably by at least 25%, even more preferably by at least 50%, yet more preferably by at least 75%, and most preferably by at least 90%, compared to untreated cardiomyocytes.

By “exercise tolerance” is meant the capacity of a subject to perform physical exercise at a duration and/or level that would normally be expected for the average healthy individual. A decrease in exercise tolerance may be characterized by exercise-induced pain, fatigue, or other negative effects.

By “neuregulin” or “NRG” is meant a peptide that is encoded by an NRG-1, NRG-2, NRG-3, or NRG-4 gene or nucleic acid, e.g., a cDNA, and binds to and activates ErbB2, ErbB3, or ErbB4 receptors, or combinations thereof.

By “neuregulin-1,” “NRG-1,” “heregulin,” “GGF2,” or “p185erbB2 ligand” is meant a peptide that binds to the ErbB2 receptor when paired with another receptor (ErbB1, ErbB3 or ErbB4) and is encoded by the p185erbB2 ligand gene described in U.S. Pat. No. 5,530,109; U.S. Pat. No. 5,716,930; and U.S. Pat. No. 7,037,888, each of which is incorporated herein by reference in its entirety.

By “neuregulin-like peptide” is meant a peptide that possesses an EGF-like domain encoded by a neuregulin gene, and binds to and activates ErbB2, ErbB3, ErbB4, or a combination thereof.

By “epidermal growth factor-like domain” or “EGF-like domain” is meant a peptide motif encoded by the NRG-1, NRG-2, NRG-3, or NRG-4 gene (or cDNA) that binds to and activates ErbB2, ErbB3, ErbB4, or combinations thereof, and bears a structural similarity to the EGF receptor-binding domain as disclosed in Holmes et al., Science 256:1205-1210, 1992; U.S. Pat. No. 5,530,109; U.S. Pat. No. 5,716,930; U.S. Pat. No. 7,037,888; Hijazi et al., Int. J. Oncol. 13:1061-1067, 1998; Chang et al., Nature 387:509-512, 1997; Carraway et al., Nature 387:512-516, 1997; Higashiyama et al., J Biochem. 122:675-680, 1997; and WO 97/09425).

By “anti-ErbB2 antibody” or “anti-HER2 antibody” is meant an antibody that specifically binds to the extracellular domain of the ErbB2 (also known as HER2 in humans) receptor and prevents the ErbB2 (HER2)-dependent signal transduction initiated by neuregulin binding.

By “transformed cell” is meant a cell (or a descendent of a cell) into which a DNA molecule encoding a peptide, e.g., a peptide comprising an EGF-like domain, e.g., a neuregulin, such as a GGF2 or a functional fragment thereof, has been introduced, by means of recombinant DNA techniques or known gene therapy techniques.

By “promoter” is meant a minimal sequence sufficient to direct transcription. Also included in the disclosure are those promoter elements which are sufficient to render promoter-dependent gene expression controllable based on cell type or physiological status, e.g., hypoxic versus normoxic conditions, or inducible by external signals or agents; such elements may be located in the 5′ or 3′ or internal regions of the native gene.

By “operably linked” is meant that a nucleic acid, e.g., a cDNA, encoding a peptide and one or more regulatory sequences are connected in such a way as to permit gene expression when the appropriate molecules, e.g., transcriptional activator proteins, are bound to the regulatory sequences.

By “expression vector” is meant a genetically engineered plasmid or virus, derived from, for example, a bacteriophage, adenovirus, retrovirus, poxvirus, herpesvirus, or artificial chromosome, that is used to transfer a peptide, e.g., a peptide comprising an EGF-like domain, e.g., a neuregulin, such as a GGF2 or a functional fragment thereof, coding sequence, operably linked to a promoter, into a host cell, such that the encoded peptide is expressed within the host cell.

Unless defined otherwise, all technical and scientific terms used herein have the same meaning as commonly understood by one of ordinary skill in the art to which this disclosure belongs.

The patent and scientific literature referred to herein establishes the knowledge that is available to those with skill in the art. All United States patents and published or unpublished United States patent applications cited herein, including US 2011/0166068, are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this disclosure has been particularly shown and described with references to preferred embodiments thereof, it will be understood by those skilled in the art that various changes in form and details may be made therein without departing from the scope of the disclosure encompassed by the appended claims.

The following Examples will assist those skilled in the art to better understand the disclosure and its principles and advantages. It is intended that these Examples be illustrative of the disclosure and not limit the scope thereof.

### EXAMPLES

### Example 1

### General Materials and Methods

### Cloning, Expression and Purification of the IgEGF (Ig154Y) Domain of GGF2 (EGF-Ig) DNA

IgEGF domain was amplified from an existing GGF2 cDNA and cloned into pet 15b vector (Novagen cat #69661-3) using Nde1 and BamH1 restriction sites. The resulting protein was 21.89 kDa+˜3 kDa His tag (=˜25 kDa).

DNA sequence of IgEgf pet 15 clone (SEQ ID NO: 26): The underlined sequences were the primers used for amplification. The bolded sequences were the cloning sites used to insert the sequence into the pet vector (Nde1 and BamH1). The translated amino acid sequence (SEQ ID NO: 27) of the IgEgf pet 15 DNA sequence is also shown below.

```
(SEQ ID NO: 26)
CATATGttgcctccccaattgaaagagatgaaaagccaggaatcggctgcaggttccaaa
              L P P Q L K E M K S Q E S A A G S K
ctagtccttcggtgtgaaaccagttctgaatactcctctctcagattcaagtggttcaag
  L V L R C E T S S E Y S S L R F K W F K
aatgggaatgaattgaatcgaaaaaacaaaccacaaaatatcaagatacaaaaaaagcca
  N G N E L N R K N K P Q N I K I Q K K P
gggaagtcagaacttcgcattaacaaagcatcactggctgattctggagagtatatgtgc
  G K S E L R I N K A S L A D S G E Y M C
aaagtgatcagcaaattaggaaatgacagtgcctctgccaatatcaccatcgtggaatca
  K V I S K L G N D S A S A N I T I V E S
aacgctacatctacatccaccactgggacaagccatcttgtaaaatgtgcggagaaggag
  N A T S T S T T G T S H L V K C A E K E
aaaactttctgtgtgaatggaggggagtgcttcatggtgaaagacctttcaaacccctcg
  K T F C V N G G E C F M V K D L S N P S
agatacttgtgcaagtgcccaaatgagtttactggtgatcgctgccaaaactacgtaatg
  R Y L C K C P N E F T G D R C Q N Y V M
gccagcttctacGGATCC
(SEQ ID NO: 27)
  A S F Y
```

The final translated protein from pet 15b vector containing the DNA sequence of IgEgf is shown below (SEQ ID NO: 28). The vector portion is underlined.

M G G S H H H H H H G M A S M T G G T A N G
V G D L Y D D D D K V P G S L P P Q L K E M
K S Q E S A A G S K L V L R C E T S S E Y S
S L R F K W F K N G N E L N R K N K P Q N I
K I Q K K P G K
S E L R I N K A S L A D S G E Y M C K V I S
K L E N D S A S A N I T I V E S N A T S T S
T T G T S H L V K C A E K E K T F C V N G G
E C F M V K D L S N P S R Y L C K C P N E F
T G D R C Q N Y V M A S F Y

Protein expression: The clone was transformed into B121 cells for protein expression using the Overnight Express Autoinduction System (Novagen) in LB media at 25° C. for 24 hours.

Protein Refolding: Adapted from Novagen Protein Refolding Kit, 70123-3.

Protein Purification: His TRAP columns—as per manufacturer's instructions.

Western blotting: Protein expression was assessed by western blotting. Resulting band with the His tag runs at around 25 kD. A 4-20% criterion gel (Biorad) was used for protein resolution followed by transfer onto Protran nitrocellulose paper (0.1 μm pore size from Schliecher and Schull). The blot was blocked in 5% milk in TBS-T (0.1%). Primary antibody (Anti EGF Human NRGI-alpha/HRG1-alpha Affinity Purified Polyclonal Ab Cat # AF-296-NA from R&D systems) 1:1000 dilution in 5% milk in TBS-T-1 hour at RT (also works at 4° C. overnight). Rabbit anti goat HRP secondary antibody was used at 1:10,000 dilution in 5% milk in TBS-T for 1 hour at RT. All washes were performed in TBS-T.

### Purification Protocol for Ig154Y

The cultures were grown at 25° C. in Overnight Express Autoinduction System 1 from Novagen (cat#71300-4). The culture was spun down and the pellets were extracted, solubilized and re-folded to acquire the Igl54Y before purification can take place.

Materials for extraction, solubilization and re-folding:

### 10× Wash Buffer: 200 mM Tris-HCl, pH 7.5, 100 mM EDTA, 10% Triton X-100

### 10× Solubilization Buffer: 500 mM CAPS, pH 11.0

### 50× Dialysis Buffer: 1M Tris-HCl, pH 8.5

30% N-laurylsarcosine—add as powder (Sigma 61739-5G)

### 1M DTT

Reduced glutathione (Novagen 3541) Oxidized glutathione (Novagen 3542)

Protocol for Cell Lysis and Preparation of Inclusion Bodies:

Step 1—Cell pellets were thawed and re-suspended in 30 mls 1× wash buffer.
Step 2—Protease inhibitors (25 μl of 10× per 50 mls), DNase (200 μl of 1 mg/ml per 50 ml) and MgCl₂ (500 μl of 1M per 50 mls) were added to suspension.
Step 3—Cells were lysed by sonication with cooling on ice.
Step 4—Following sonication inclusion bodies were collected by centrifugation at 10,000×g for 12 minutes.
Step 5—Supernatant was removed and the pellet thoroughly re-suspended in 30 mls of 1× Wash Buffer.
Step 6—Step 4 was repeated.
Step 7—The pellet was thoroughly re-suspended in 30 mls of 1× Wash Buffer.
Step 8—The inclusion bodies were collected by centrifugation at 10,000×g for 10 minutes.

Protocol for Solubilization and Refolding:

Step 1—From the wet weight of inclusion bodies to be processed, the amount of 1× Solubilization Buffer necessary to re-suspend the inclusion bodies at a concentration of 10-15 mg/ml was calculated. If the calculated volume was greater than 250 ml, 250 ml was used.
Step 2—At room temperature, prepared the calculated volume of 1× Solubilization Buffer supplemented with 0.3% N-laurylsarcosine (up to 2% could be used if needed in further optimization) (300 mg/100 mL buffer) and 1 mM DTT.
Step 3—Added the calculated amount of 1× Solubilization Buffer from step 2 to the inclusion bodies and gently mixed. Large debris could be broken up by repeated pipetting.
Step 4—Incubated in refrigerator shaker at 25° C., 50-100 rpm for 4-5 hours (or longer if needed in further optimization).
Step 5—Clarified by centrifugation at 10,000×g for 10 minutes at room temperature
Step 6—Transferred the supernatant containing the soluble protein into a clean tube.

Protocol for Dialysis Protocol for Protein Refolding

Step 1—Prepared the required volume of buffer for dialysis of solubilized protein. The dialysis was performed with at least 2 buffer changes of greater than 50 times the volume of the sample. Diluted the 50× Dialysis Buffer to 1× at the desired volume and supplemented with 0.1 mM DTT.
Step 2—Dialyzed for at least 4 hours at 4° C. Changed the buffer and continued. Dialyzed for an additional 4 or more hours.
Step 3—Prepared additional dialysis buffer as determined in step 1, but omit DTT.
Step 4—Continued the dialysis through two additional changes (4 hr each), with the dialysis buffer lacking DTT

Protocol for Redox Refolding Buffer to Promote Disulfide Bond Formation

Step 1—Prepared a dialysis buffer containing 1 mM reduced glutathione (1.2 g/4 L) and 0.2 mM oxidized glutathione (0.48 g/4 L) in 1× Dialysis Buffer. The volume was 25 times greater than the volume of the solubilized protein sample. Chilled to 4° C.
Step 2—Dialyzed the refolded protein from step 1 overnight at 4° C.

Protein purification materials:

All procedures were done at 4° C.

Chemicals:
Trizma Hydrochloride (Sigma T5941-500G)
Sodium Chloride 5M Solution (Sigma 56546-4 L)
Sodium Hydroxide ION (JT Baker 5674-02)
Imidazole (JT Baker N811-06)

Protocol for Purification on the HISPrep FF 16/10 Column-20 mls (GE Healthcare)

### Buffer A: 20 mM Tris-HCL+500 mM NaCl pH 7.5

### Buffer B: Buffer A+500 mM Imidazole pH 7.5

Step 1—Equilibration of column: Buffer A—5CV, Buffer B—5CV, Buffer A—10CV
Step 2—Loaded 20 ml of sample per run on 20 ml column at 0.5 ml/min
Step 3—Washed column with 5CV of buffer A
Step 4—Eluted column with 5CV of 280 mM Imidazole.
Step 5—Cleaned with 10CV of 100% Buffer B.
Step 6—Equilibrated with 15CV of Buffer A
Step 7—Analyzed fractions with a SDS-page silver stain Pool fractions with Igl54Y

His-Tag Removal

Removal of the His-Tag was done with A Thrombin Cleavage Capture Kit from Novagen (Cat#69022-3). Based on previous testing, the best conditions were room temperature for 4 hours with Thrombin at 0.005U of enzyme per μl for every 10 μg of Ig154Y protein. After four hours of incubation, added 16 μl of Streptavidin Agarose slurry per unit of Thrombin enzyme. Rocked sample for 30 minutes at room temp. Recovered the Ig154Y through spin-filtration or sterile filtering (depending on volume). Full cleavage was determined by EGF and Anti-His Western blotting.

Concentration of Ig154Y

Adjusted to desired concentration with Millipore Centriprep 3000 MWCO 15 ml concentrator (Ultracel YM-3, 4320)

Storage in Final Buffer

### Stored in 20 mM Tris+500 mM NaCl pH 7.5 and 1×PBS+0.2% BSA.

### Cloning, Expression and Purification of 156Q (EGF-Id) [NRG1b2 EGF Domain (156Q)]

DNA: NRG1b2 egf domain was cloned from human brain cDNA and cloned into pet 15b vector (Novagen cat #69661-3) using Nde 1 and BamH1 restriction sites. The resulting protein was 6.92 kda+˜3 kDa His tag (=9.35 kDa).

DNA sequence of NRG1b2 egf pet 15 clone (SEQ ID NO: 29). The underlined sequences are the cloning sites (Nde1 and BamH1)

CATATGAGCCA TCTTGTAAAA TGTGCGGAGA AGGAGAAAAC
TTTCTGTGTG AATGGAGGGG AGTGCTTCAT GGTGAAAGAC
CTTTCAAACC CCTCGAGATA CTTGTGCAAG TGCCCAAATG
AGTTTACTGG TGATCGCTGC CAAAACTACG TAATGGCCAG
CTTCTACAAG GCGGAGGAGC TGTACCAGTA AGGATCC

The final translated protein from pet15b vector containing the NRG1b2 egf DNA sequence above is shown below (SEQ ID NO: 30). The egf domain is underlined.

MGSSHHHHHH SSGLVPRGSH MSHLVKCAEK EKTFCVNGGE
CFMVKDLSNP SRYLCKCPNE FTGDRCQNYV MASFYKAEEL YQ

Calculated pI/Mw: 7.69/9349.58

Protein expression: The clone was transformed into BL21 cells for protein expression using the Overnight Express Autoinduction System (Novagen) in LB media at 25° C. for 24 hours. Expression was primarily in insoluble inclusion bodies.

Protein Refolding: Adapted from Novagen Protein Refolding Kit, 70123-3.

Protein Purification: Protein was loaded onto an anion exchange column DEAE at 2.5 ml/min. The EGF-Id fragment remained in the flow through, whereas the contaminants bound and eluted at a higher salt. The loading and washing buffer was 50 mM Tris pH7.9 and elution buffer was 50 mM Tris pH7.9 with 1M NaCl. The flow through was pooled and concentrated with Centriprep YM-3 from Millipore.

Western blotting: Protein expression was assessed by Western blotting. Resulting band ran at around 10 kD. A 4-20% criterion gel (Biorad) was used for protein resolution followed by transfer onto Protran nitrocellulose paper (0.1 μm pore size from Schliecher and Schull). The blot was blocked in 5% milk in TBS-T (0.1%). Primary antibody (Anti EGF Human NRG1-alpha/HRG1-alpha Affinity. Purified Polyclonal Ab Cat # AF-296-NA from R&D systems) 1:1000 dilution in 5% milk in TBS-T for 1 hour at RT (also worked at 4° C. overnight). Rabbit anti goat HRP secondary antibody was used at 1:10,000 dilution in 5% milk in TBS-T for 1 hour at RT. All washes were performed in TBS-T.

### Purification Protocol for NRG-156Q

Cultures were grown at 25° C. in Overnight Express Autoinduction System 1 from Novagen (cat#71300-4). There was very little soluble NRG-156Q (EGF-Id) present. The culture was spun down and the pellets were extracted, solubilized and re-folded to acquire the NRG-156Q before purification could take place.

Materials for extraction, solubilization and re-folding:
10× Wash Buffer: 200 mM Tris-HCl, pH 7.5, 100 mM EDTA, 10% Triton X-100
10× Solubilization Buffer: 500 mM CAPS, pH 11.0
50× Dialysis Buffer: 1M Tris-HCl, pH 8.5
30% N-laurylsarcosine—add as powder (Sigma 61739-5G)
1M DTT
Reduced glutathione (Novagen 3541) Oxidized glutathione (Novagen 3542)

Cell Lysis and Preparation of Inclusion Bodies

Step 1—Thawed and re-suspended cell pellet in 30 mls 1× wash buffer. Mixed as needed for full re-suspension.
Step 2—Added protease inhibitors (25 μl of 10× per 50 mls), DNase (200 μl of 1 mg/ml per 50 ml) and MgCl₂ (500 μl of 1M per 50 mls) to suspension.
Step 3—Lysed the cells by sonication.
a. Cooled the cells on ice throughout this step.
b. Using the square tip, sonicated for 30 seconds on level 6, 10 times until suspension became less viscous. Let suspension cool on ice for 60 seconds between each sonication. Kept volume no higher than 40 mls in 50 ml conical tube when sonicating.
Step 4—When complete, transferred each suspension to 250 ml angled neck centrifuge bottles for use with F-16/250 rotor.
Step 5—Collected the inclusion bodies by centrifugation at 10,000×g for 12 minutes.
Step 6—Removed the supernatant (saved a sample for analysis of soluble protein) and thoroughly re-suspended the pellet in 30 mls of 1× Wash Buffer.
Step 7—Repeated centrifugation as in Step 4 and saved the pellet.
Step 8—Again, thoroughly re-suspended the pellet in 30 mls of 1× Wash Buffer.
Step 9—Collected the inclusion bodies by centrifugation at 10,000×g for 10 minutes.
Decanted the supernatant and removed the last traces of liquid by tapping the inverted tube on a paper towel.

Solubilization and Refolding

Step 1—From the wet weight of inclusion bodies to be processed, the amount of 1× Solubilization Buffer necessary to re-suspend the inclusion bodies at a concentration of 10-15 mg/ml was calculated. If the calculated volume was greater than 250 ml, 250 ml was used.
Step 2—At room temperature, prepared the calculated volume of 1× Solubilization Buffer supplemented with 0.3% N-laurylsarcosine (up to 2% could be used if needed in further optimization) (300 mg/100 mL buffer) and 1 mM DTT.
Step 3—Added the calculated amount of 1× Solubilization Buffer from step 2 to the inclusion bodies and gently mixed. Large debris could be broken up by repeated pipetting.
Step 4—Incubated in refrigerator shaker at 25° C., 50-100 rpm for 4-5 hours.
Step 5—Clarified by centrifugation at 10,000×g for 10 minutes at room temperature.

Dialysis Protocol for Protein Refolding

Step 1—Prepared the required volume of buffer for dialysis of solubilized protein. The dialysis was performed with at least 2 buffer changes of greater than 50 times the volume of the sample.
Step 2—Diluted the 50× Dialysis Buffer to 1× at the desired volume and supplemented with 0.1 mM DTT.
Step 3—Dialyzed for at least 4 hours at 4° C. Changed the buffer and continued. Dialyzed for an additional 4 or more hours.
Step 4—Prepared additional dialysis buffer as determined in step 1, but omit DTT.
Step 5—Continued the dialysis through two additional changes (4 hours each), with the dialysis buffer lacking DTT.

Redox Refolding Buffer to Promote Disulfide Bond Formation

Step 1—Prepared a dialysis buffer containing 1 mM reduced glutathione (1.2 g/4 L) and 0.2 mM oxidized glutathione (0.48 g/4 L) in 1× Dialysis Buffer. The volume was 25 times greater than the volume of the solubilized protein sample. Chilled to 4° C.
Step 2—Dialyzed the refolded protein from step 1 overnight at 4° C.

Materials for Purification

All procedures were done at 4° C.

### Chemicals:

Trizma Hydrochloride (Sigma T5941-500G)
Sodium Chloride 5M Solution (Sigma 56546-4L)
Sodium Hydroxide ION (JT Baker 5674-02)

Purification on the DEAE HiPrep 16/10 Anion Column—20 mls (GE Healthcare)

### Buffer A: 50 mM Tris-HCL pH 8.0

Buffer B: 50 mM Tris-HCL with 1M NaCl pH 8.0
Step 1—Equilibration of column: Buffer A—5CV, Buffer B—5CV, Buffer A—10CV
Step 2—Loaded 50 ml of sample per run on 20 ml column at 2.0 ml/min (NRG-156 (EGF-Id) was in the flow through).
Step 3—Washed 20 ml column with 5CV of buffer A
Step 4—Used 20 ml column with gradient to 100% B with 5CV to elute off contaminants
Step 5—Cleaned with 10CV of 100% Buffer B
Step 6—Equilibrated with 15CV of Buffer A
Step 7—Analyzed fractions with a SDS-page silver stain
Step 8—Pooled fractions with NRG-156Q (10 kDa)

Concentration of NRG-156 (EGF-Id)

Step 1—Concentrated with Millipore Centriprep 3000 MWCO 15 ml concentrator (Ultracel YM-3, 4320)
Step 2—Used Modified Lowry Protein Assay to determine concentration.

His-Tag Removal

Removal of the His-Tag was done with A Thrombin Cleavage Capture Kit from Novagen (Cat#69022-3). Based on previous testing the best conditions were room temperature for 4 hours with Thrombin at 0.005U of enzyme per μl for every 10 μg of NRG-156Q (EGF-Id) protein. After four hours of incubation, added 16 μl of Streptavidin Agarose slurry per unit of Thrombin enzyme. Rocked sample for 30 minutes at room temperature. Recovered the NRG-156Q through spin-filtration or sterile filtering (depending on volume). Complete cleavage was determined with an EGF and Anti-His Western.

Storage in final buffer: Stored in 1×PBS with 0.2% BSA at 4° C.

### Expression and Purification of GGF2

For the cloning and background information for GGF2, see U.S. Pat. No. 5,530,109. The cell line is described in U.S. Pat. No. 6,051,401. The entire contents of each of U.S. Pat. No. 5,530,109 and U.S. Pat. No. 6,051,401 are incorporated herein by reference.

CHO-(Alpha2HSG)-GGF cell line: This cell line was designed to produce sufficient quantities of fetuin (human alpha2HSG) to support high production rates of rhGGF2 in serum free conditions.

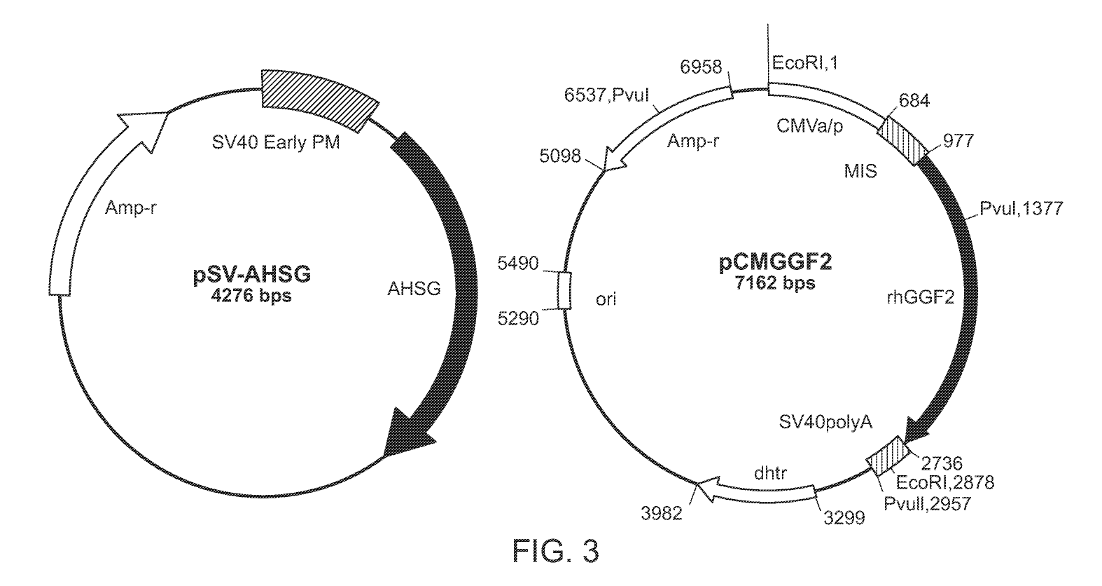

CHO (dhfr−) cells were transfected with the expression vector shown below (pSV-AHSG). Stable cells were grown under ampicillin selection. The cell line was designated (dhfr−/α2HSGP). The dhfr−/α2HSGP cells were then transfected with the pCMGGF2 vector shown in FIG. 3 containing the coding sequence for human GGF2 using the cationic lipid DMRIE-C reagent (Life Technologies #10459-014).

Stable and high producing cell lines were derived under standard protocols using methotrexate (100 nM, 200 nM, 400 nM, 1 μM) at 4-6 weeks intervals. The cells were gradually weaned from serum containing media. Clones were isolated by standard limiting dilution methodologies. Details of the media requirements are described herein.

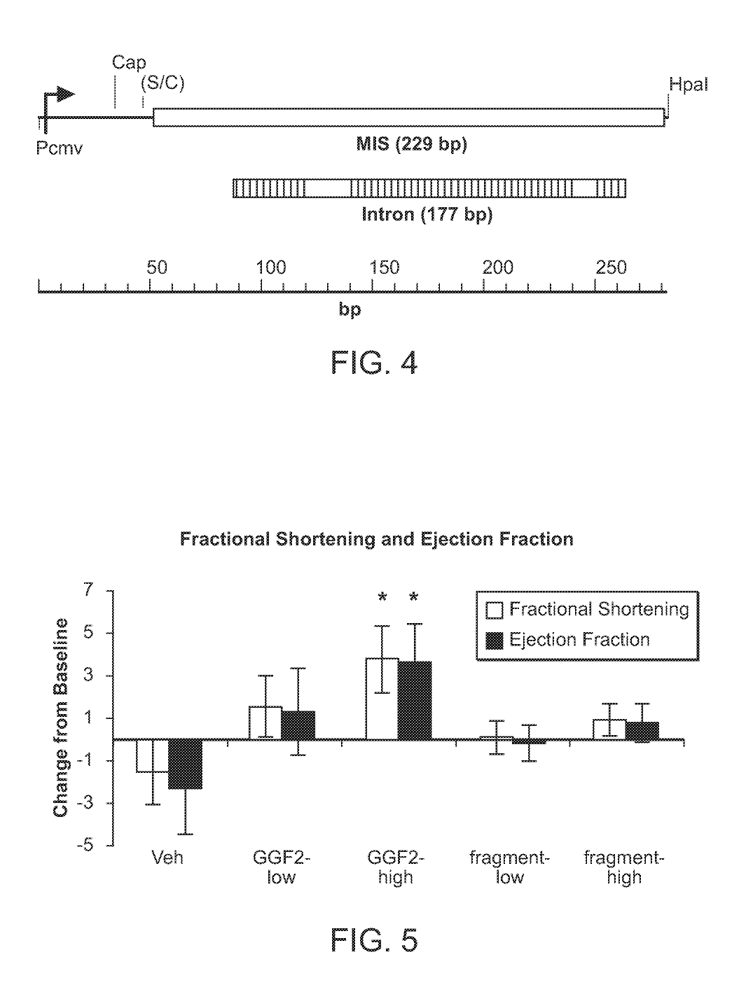

To enhance transcription, the GGF2 coding sequence was placed after the EBV BMLF-1 intervening sequence (MIS). See FIG. 4.

MIS Sequence (SEQ ID NO: 31)

CGAT[AACTAGCAGCATTTCCTCCAACGAGGATCCCGCAG
(GTAAGAAGCTACACCGGCCAGTGGCCGGGGCC
CGATAACTAGCAGCATTTCCTCCAACGAGGATCCCGCAG(GTAAGAAGCT
ACACCGGCCAGTGGCCGGGGCC
GTGGAGCCGGGGGCATCCGGTGCCTGAGACAG AGGTGCTCAAGGCAGTC
TCCACCTTTTGTCTCCCCTCTGCAG)AGAGCCACATTCTGGAA]GTT

GGF2 coding sequence (SEQ ID NO: 3)

atgagatgg cgacgcgccc cgcgccgctc cgggcgtccc
ggcccccggg cccagcgccc cggctccgcc gcccgctcgt
cgccgccgct gccgctgctg ccactactgc tgctgctggg
gaccgcggcc ctggcgccgg gggcggcggc cggcaacgag
gcggctcccg cgggggcctc ggtgtgctac tcgtccccgc
ccagcgtggg atcggtgcag gagctagctc agcgcgccgc
ggtggtgatc gagggaaagg tgcacccgca gcggcggcag
cagggggcac tcgacaggaa ggcggcggcg gcggcgggcg
aggcaggggc gtggggcggc gatcgcgagc cgccagccgc
gggcccacgg gcgctggggc cgcccgccga ggagccgctg
ctcgccgcca acgggaccgt gccctcttgg cccaccgccc
cggtgcccag cgccggcgag cccggggagg aggcgcccta
tctggtgaag gtgcaccagg tgtgggcggt gaaagccggg
ggcttgaaga aggactcgct gctcaccgtg cgcctgggga
cctggggcca ccccgccttc ccctcctgcg ggaggctcaa
ggaggacagc aggtacatct tcttcatgga gcccgacgcc
aacagcacca gccgcgcgcc ggccgccttc cgagcctctt
tcccccctct ggagacgggc cggaacctca agaaggaggt
cagccgggtg ctgtgcaagc ggtgcgcctt gcctccccaa
ttgaaagaga tgaaaagcca ggaatcggct gcaggttcca
aactagtcct tcggtgtgaa accagttctg aatactcctc
tctcagattc aagtggttca agaatgggaa tgaattgaat
cgaaaaaaca aaccacaaaa tatcaagata caaaaaaagc
cagggaagtc agaacttcgc attaacaaag catcactggc
tgattctgga gagtatatgt gcaaagtgat cagcaaatta
ggaaatgaca gtgcctctgc caatatcacc atcgtggaat
caaacgctac atctacatcc accactggga caagccatct
tgtaaaatgt gcggagaagg agaaaacttt ctgtgtgaat
ggaggggagt gcttcatggt gaaagacctt tcaaacccct
cgagatactt gtgcaagtgc ccaaatgagt ttactggtga
tcgctgccaa aactacgtaa tggccagctt ctacagtacg
tccactccct ttctgtctct gcctgaatag

Full length human GGF2 Protein Sequence (SEQ ID NO: 1)

MRWRRAPRRSGRPGPRAQRPGSAARSSPPLPLLPLLLLLGTAALAPGAAA
GNEAAPAGASVCYSSPPSVGSVQELAQRAAVVIEGKVHPQRRQQGALDRK
AAAAAGEAGAWGGDREPPAAGPRALGPPAEEPLLAANGTVPSWPTAPVPS
AGEPGEEAPYLVKVHQVWAVKAGGLKKDSLLTVRLGTWGHPAFPSCGRLK
EDSRYIFFMEPDANSTSRAPAAFRASFPPLETGRNLKKEVSRVLCKRCAL
PPQLKEMKSQESAAGSKLVLRCETSSEYSSLRFKWFKNGNELNRKNKPQN
IKIQKKPGKSELRINKASLADSGEYMCKVISKLGNDSASANITIVESNAT
STSTTGTSHLVKCAEKEKTFCVNGGECFMVKDLSNPSRYLCKCPNEFTGD
RCQNYVMASFYSTSTPFLSLPE

GGF2 production: One vial of GGF2 at 2.2×10⁶ cells/mL was thawed into 100 mls of Acorda Medium 1 (see Table 1) and expanded until reaching sufficient numbers to seed production vessels. Cells were inoculated into the production media Acorda Medium 2 (see Table 2) at 1.0×10⁵ cells/mL in two liter vented roller bottles. Roller bottles were maintained at 37° C. for 5 days and then reduced to 27° C. for 26 days. The roller bottles were monitored for cell count and general appearance but they are not fed. Once viability was below 10%, the cells were spun out and conditioned media harvested and sterile filtered.

TABLE 1
Medium 1

| | | Catalog | | |
|---|---|---|---|---|
| Item | Vendor | Number | Final concentration | |
| CD-CHO | Invitrogen | 10743-029 | remove 50 ml, then | |
| | | | add components | |
| | | | below | |
| FeSO₄•EDTA | Sigma | F-0518 | 1x | (10 ml/L) |
| L-Glutamine | Cellgro | 25-005-CI | 4 mM | (20 ml/L) |
| Recombinant | Sigma | 1-9278 | 290 U/L | (1 ml/L) |
| Human Insulin | | | | |
| Non-essential | Cellgro | 25-025-C1 | 1x | (10 ml/L) |
| amino acid | | | | |
| Peptone Type 4 | Sigma | P0521 | Powder -- Made 20X | |
| Soybean-HySoy | | | in CD-CHO (50 ml/L) | |
| Gentamicin | Invitrogen | 15750-078 | 100 μg | (2 ml/L) |

TABLE 2
Medium 2

| | | Catalog | | |
|---|---|---|---|---|
| Item | Vendor | Number | Final concentration | |
| CD-CHO | Invitrogen | 10743-029 | 50% | (−50 ml first) |
| HyQ SFX-CHO | HyClone | SH30187.02 | 50% | (−50 ml first) |
| FeSO₄•EDTA | Sigma | F-0518 | 1x | (10 ml/L) |
| L-Glutamine | Cellgro | 25-005-CI | 4 mM | (20 ml/L) |
| Recombinant | Sigma | 1-9278 | 290 U/L | (1 ml/L) |
| Human Insulin | | | | |
| Non-essential | Cellgro | 25-025-CI | 1x | (10 ml/L) |
| amino acid | | | | |
| Peptone Type 4 | Sigma | P0521 | Powder -- Made 20X in | |
| Soybean-HySoy | | | CD-CHO (50 ml/L) | |
| Gentamicin | Invitrogen | 15750-078 | 100 μg | (2 ml/L) |

Purification Protocol for GGF2

All procedures were done at 4° C.

### Chemicals:

Sodium Acetate
Glacial Acetic Acid (for pH adjustment)
10N NaOH (for pH adjustment)
NaCl
Sodium Sulfate
L-Arginine (JT Baker cat #: 2066-06)
Mannitol (JT Baker cat #: 2553-01)

Starting material: Conditioned media supernatant. Adjusted pH to 6.5.

Step 1:

Capture—Cation Exchange Chropmatography
HiPrep SP 16/10 (Amersham Biosciences)
Column equilibration: Buffer A—5CV, buffer B—5CV, buffer 15% B—5CV
Buffer A: 20 mM NaAcetate, pH 6.0
Buffer B: 20 mM NaAcetate, pH 6.0, 1M NaCl

Loaded sample at 2 ml/min with a continuous load overnight if possible. Binding was better with continuous loading.

Maximum capacity for a starting sample: 5 mg GGF2/ml media
Flow rate: 3 ml/min
First wash: 15% B, 10CV
Second wash: 35% B, 10CV
GGF2 elution: 60% B, 8CV
Column wash: 100% B, 8CV

Buffers

| Buffers | Composition | Conductivity | Use |
|---|---|---|---|
| 15% B | 20 mM NaAcetate, | | Preequilibrium and |
| | pH 6.0, 150 mM NaCl | | First Wash |
| 35% B | 20 mM NaAcetate, | | Second Wash |
| | pH 6.0, 350 mM NaCl | | |
| 60% B | 20 mM NaAcetate, | | GGF2 elution |
| | pH 6.0, 600 mM NaCl | | |
| 100% B | 20 mM NaAcetate, | 88 mS/cm | Column Wash |
| | pH 6.0, 1000 mM NaCl | | |

Step 2:

### Refinement—Gel Filtration Chromatography

Sephacryl S200 26/60
Elution buffer: 20 mM NaAcetate, 100 mM Sodium Sulfate, 1% Mannitol, and 10 mM L-Arginine, pH 6.5
Buffer conductivity:
Sample: SP GGF2 elution pool concentrated up to ˜AU280 1.0
Flow rate: 1.3 ml/min
   Peak elution: at ˜0.36CV from injection start

Step 3—DNA and Endotoxin removal by filtration through Intercept Q membrane.

Preequilibration buffer: 20 mM NaAcetate, 100 mM Sodium Sulfate, 1% Mannitol, and 10 mM L-Arginine, pH 6.5

Collected flow through

Step 4—Final formulation and sample preparation
Added additional 90 mM L-Arginine to the sample
Concentrated
Sterile Filtered

The vehicle/control article used herein was 0.2% Bovine Serum Albumin (BSA), 0.1 M Sodium Phosphate, pH 7.6.

Rat strains CD®IGS [Crl:CD (SD)/MYOINFARCT] and naïve Sprague Dawley are used herein. These strains were acquired from Charles River Laboratories. The test animals were approximately 6-7 weeks of age at arrival and weighed approximately 160-200 grams, at the time of surgical procedure. The actual range may vary.

All naïve Sprague Dawley animals received were placed on study and assigned to Group 1. Animals considered suitable for study were weighed prior to treatment.

All CD®IGS [Crl:CD (SD)/MYOINFARCT] animals received were randomized into treatment groups (Groups 2-5) using a simple randomization procedure based on calculated Ejection Fraction from Echocardiographic examinations performed on Day 7 post-surgical procedure conducted at Charles River Laboratories. Simple randomization was conducted to result in each treatment group (Groups 2-5) consisting of applicable numbers of animals resulting in an approximately equal Group Mean Ejection Fraction (±3%) across Group 2-5.

All animals in Group 2-6 were acclimated at Charles River Laboratories according to Standard Operating Procedures of that laboratory. Animals were subsequently randomized into treatment groups. All naïve animals in Group 1 were acclimated for approximately 24 hours post receipt prior to their primary echocardiographic examinations.

The animals were individually housed in suspended, stainless steel, wire-mesh type cages. Solid-bottom cages were not used in general because rodents are coprophagic and the ingestion of feces containing excreted test article and metabolic products or ingestion of the bedding itself could confound the interpretation of the results in this toxicity study.

Fluorescent lighting was provided via an automatic timer for approximately 12 hours per day. On occasion, the dark cycle was interrupted intermittently due to study-related activities. Temperature and humidity were monitored and recorded daily and maintained to the maximum extent possible between 64 to 79° F. and 30 to 70%, respectively.

The basal diet was block Lab Diet® Certified Rodent Diet #5002, PMI Nutrition International, Inc. This diet was available ad libitum unless designated otherwise. Each lot number used was identified in the study records. Tap water was supplied ad libitum to all animals via an automatic water system unless otherwise indicated.

### Example 2

### Animal Model Study Designs and Evaluation

TABLE 3
GGF2 versus EGF-Id fragment (Liu et al. J. Am. Coll. Cardiol.
48.7(2006): 1438-47) dosed for 10 days starting day 7 after LAD

| | | In-Life | | Dosing | ECHO Time |
|---|---|---|---|---|---|
| Group | Treatment | Duration | Dose | Interval† | Points (post-op) |
| 1 | Control | 17 days post-op | Vehicle only | 24 Hr | Day 6, 17 |
| (n = 5 M, n = 5 F) | (Vehicle) | | | | |
| 2 | GGF2 | 17 days post | 0.0625 mg/kg | 24 Hr | Day 6, 17 |
| (n = 6 M, n = 6 F) | | | | | |
| 3 | GGF2 | 17 days post | 0.625 mg/kg | 24 Hr | Day 6, 17 |
| (n = 6 M, n = 6 F) | | | | | |
| 4 | EGF-Id | 17 days post | Equimolar | 24 Hr | Day 6, 17 |
| (n = 6 M, n = 7 F) | | | | | |
| 5 | EGF-Id | 17 days post | Equimolar | 24 Hr | Day 6, 17 |
| (n = 7 M, n = 6 F) | | | | | |

TABLE 4
GGF2 higher dose compared with EGF-Id and EGF-Ig. Dosed
for 20 days starting day 7 after LAD. 10 day washout.

| | | In-Life | | Dosing | ECHO Time |
|---|---|---|---|---|---|
| Group | Treatment | Duration | Dose | Interval† | Points (post-op) |
| 1 | N/A: Age | 30 days post | NA | NA | Day 1, 12, 22, |
| (n = 5 M, n = 5 F) | Matched Naïve | primary ECHO | | | & 32 |
| | controls | | | | |
| 2 | Control (Vehicle) | 38 days post-op | Vehicle only | 24 Hr | *Day 7, 18, 28, |
| (n = 6 M, n = 6 F) | | | | | & 38 |
| 3 | GGF-2 | 38 days post-op | 0.625 mg/kg | 24 Hr | *Day 7, 18, 28, |
| (n = 6 M, n = 6 F) | | | | | & 38 |
| 4 | GGF-2 | 38 days post-op | 3.25 mg/kg | 24 Hr | *Day 7, 18, 28, |
| (n = 6 M, n = 7 F) | | | | | & 38 |
| 5 | EGF-1d | 38 days post-op | Equimolar | 24 Hr | *Day 7, 18, 28, |
| (n = 7 M, n = 6 F) | | | | | & 38 |
| 6 | EGF-Ig | 38 days post-op | Equimolar | 24 Hr | *Day 7, 18, 28, |
| (n = 7 M, n = 6 F) | | | | | & 38 |

TABLE 5
GGF2 Dose frequency

| | | In-Life | | Dosing | ECHO Time |
|---|---|---|---|---|---|
| Group | Treatment | Duration | Dose | Interval† | Points (post-op) |
| 1 | N/A: Age | 30 days post | NA | NA | :Day 1, 12, 22, |
| (n = 5 M; n = 5 F) | Matched Naïve | primary ECHO | | | & 32 |
| | controls | | | | |
| 2 | Control (Vehicle) | 38 days post-op | Vehicle only | 24 Hr | *Day 7, 18, 28, |
| (n = 6 M; n = 6 F) | | | | | & 38 |
| 3 | GGF-2 | 38 days post-op | 3.25 mg/kg | 24 Hr | *Day 7, 18, 28, |
| (n = 6 M; n = 6 F) | | | | | & 38 |
| 4 | GGF-2 | 38 days post-op | 3.25 mg/kg | 48 Hr | *Day 7, 18, 28, |
| (n = 6 M; n = 7 F) | | | | | & 38 |
| 5 | GGF-2 | 38 days post-op | 3.25 mg/kg | 96 Hr | *Day 7, 18, 28, |
| (n = 7 M; n = 6 F) | | | | | & 38 |

TA 1 = Test Article 1;
M = males;
F = females.

TABLE 6
GGF2 with and without BSA

| | | In-Life | | Dosing | ECHO Time |
|---|---|---|---|---|---|
| Group | Treatment | Duration | Dose | Interval† | Points (post-op) |
| 1 | N/A: Age | 17 days post-op | NA | NA | Day 6 and 17 |
| (n = 5 M, n = 5 F) | Matched Naive | | | | |
| | controls | | | | |
| 2 | Control (Vehicle) | 17 days post | Vehicle only | 24 Hr | Day 6 and 17 |
| (n = 6 M, n = 6 F) | | | | | |
| 3 | GGF-2 + BSA | 17 days post | 3.25 mg/kg | 24 Hr | Day 6 and 17 |
| (n = 6 M, n = 6 F) | | | | | |
| 4 | GGF-2 without | 17 days post | 3.25 mg/kg | 24 Hr | Day 6 and 17 |
| (n = 6 M, n = 7 F) | BSA | | | | |

### Test and Control Article Administration

Route of Administration: The test and control articles were administered by intravenous injection. Animals assigned to Group 1 were not treated with vehicle or Test Articles; these animals served as age matched controls without treatment. Frequency of administration, duration, and dose were as described in the Tables 3-6. The dose volume was approximately 1 ml per kg.

Test Article Administration: The test and control articles were administered via the tail vein. Individual doses were based on the most recent body weights. The dose was administered by bolus injection, unless otherwise indicated.

### Preparation of Test System

### Surgical Procedure—Left Anterior Descending Artery Ligation

The surgical procedures were performed at Charles River Laboratories as described in Charles River Laboratories Surgical Capabilities Reference Paper, Vol. 13, No. 1, 2005. Briefly, a cranio-caudal incision is made in the chest, slightly to the left of the sternum, through skin and the pectoral muscles. The third and fourth ribs are transected, and the intercostals muscles are blunt dissected. The thoracic cavity is rapidly entered, and the pericardium completely opened. The heart is exteriorized through the incision. The pulmonary cone and left auricle are identified. A small curved needle is used to pass a piece of 5-0 silk suture under the left anterior descending coronary artery. The ligature is tied, and the heart is replaced into the thorax. The air in the thoracic cavity is gently squeezed out while the thoracic wall and skin incision is closed. The animal is resuscitated using positive pressure ventilation and placed in an oxygen rich environment.

### Post-Operative Recovery

Short term post-operative monitoring and administration of appropriate analgesics were performed by Charles River Laboratories as described in Charles River Laboratories Surgical Capabilities Reference Paper, Vol. 13, No. 1, 2005. Long term post-operative monitoring was conducted to assess the animals for signs of pain or infection. Daily incision site observations continued for 7 days post receipt of animals. Supplemental pain management and antimicrobial therapy were administered as necessitated.

TABLE 7
SCHEDULED MEDICATIONS AND DOSAGES

| | INTERVAL, DOSE, AND ROUTE | | | | |
|---|---|---|---|---|---|
| | | | | | DAY 32/38* |
| | DAILY | DAY 1/7* | DAY 12/18* | DAY 22/28* | ECHO & |
| DRUG | POSTSURGERY | ECHO | ECHO | ECHO | Necropsy |
| Isoflurane | — | To effect, | To effect, | To effect, | To effect, |
| | | inhalation | inhalation | inhalation | inhalation |
| Buprenorphine | 0.01 mg/kg, I.M. | | | | |
| | (only as needed) | | | | |

*ECHO procedure Day defined by animal Group assignment as indicated below.

### Antemortem Study Evaluations

Cage-side Observations: All animals were observed at least twice a day for morbidity, mortality, injury, and availability of food and water. Any animals in poor health were identified for further monitoring and possible euthanasia.

Body Weights: Body weights were measured and recorded at least once prior to randomization and weekly during the study.

Food Consumption: Food consumption was not measured, but inappetence was documented.

Echocardiographic Examinations: Echocardiographic examinations were conducted on all animals assigned to Group 1 on Day 1, 12, 22 and Day 32 post receipt (Day 0). Echocardiographic examinations were conducted on all animals assigned to Group 2-5 on Day 7, 18, 28 and Day 38 post-surgical procedure conducted at Charles River Laboratories (Day 0).

For the echocardiographic examination, each animal was anesthetized according to Table 7 and its hair clipped from the thorax. Coupling gel was applied to the echocardiographic transducer and image obtained to measure cardiac function at multiple levels. Images were obtained for each animal in short axis view (at mid-papillary level, or other depending on location of observed infarct area by echocardiography).

Echocardiographic Parameters: ECHO images were taken at the mid-papillary muscle level, or other depending on location of observed infarct area by echocardiography, of the left ventricle. M-mode and 2-D images were recorded and stored on CD and/or MOD. Measurement parameters obtained with ECHO include: Intraventricular Septal Wall Thickness (diastole); units=cm; Intraventricular Septal Wall Thickness (systole); units=cm; Left Ventricular Internal Dimension (diastole); units=cm; Left Ventricular Internal Dimension (systole); units=cm; Left Ventricular Papillary Wall Thickness (diastole); units=cm; Left Ventricular Papillary Wall Thickness (systole); units=cm; End Diastolic Volume; units=mL; End Systolic Volume; units=mL; Ejection Fraction; reported as a percentage; Stroke Volume; units=ml; and Percent Fractional Shortening; reported as a percentage

### Euthanasia

Moribundity: Any moribund animals, as defined by a Testing Facility Standard Operating Procedure, were euthanized for humane reasons. All animals euthanized in extremis or found dead were subjected to a routine necropsy.

Method of Euthanasia: Euthanasia was performed by saturated potassium chloride injection into the vena cava followed by an approved method to ensure death, e.g. exsanguination.

Final Disposition: All surviving animals placed on study were euthanized at their scheduled necropsy or, if necessary, euthanized in extremis.

### Example 3

### Animal Study Results

The neuregulins are a family of growth factors structurally related to Epidermal Growth Factor (EGF) and are essential for the normal development of the heart. Evidence suggests that neuregulins are a potential therapeutic for the treatment of heart disease including heart failure, myocardial infarction, chemotherapeutic toxicity and viral myocarditis.

The studies described in Example 2 were served to define dosing in the left anterior descending (LAD) artery ligation model of congestive heart failure in the rat. Multiple neuregulin splice variants were cloned and produced. A neuregulin fragment of consisting of the EGF-like domain (EGF-Id) from previous reports (Liu et al., 2006) was compared to a full-length neuregulin known as glial growth factor 2 (GGF2) and the EGF-like domain with the Ig domain (EGF-Ig). Male and female Sprague-Dawley rats underwent LAD artery ligation. At 7 days post ligation rats were treated intravenously (iv) with neuregulin daily. Cardiac function was monitored by echocardiography.

The first study compared 10 days of dosing with equimolar amounts of EGF-Id or GGF2 (for GGF2 this calculates to 0.0625 and 0.325 mg/kg). GGF2 treatment resulted in significantly (p<0.05) greater improvement in Ejection Fraction (EF) and Fractional Shortening (FS) than did EGF-Id at the end of the dosing period. The second study compared 20 days of GGF2 with EGF-ld. and EGF-Ig at equimolar concentrations. GGF2 treatment resulted in significantly improved EF, FS and LVESD (p<0.01). Improvements in cardiac physiology were not maintained for this period with either EGF-ld. or EGF-Ig. The third study compared daily (q 24 hour), every other day (q 48 hour) and every fourth day (q 96 hour) dosing for 20 days with GGF2 (3.25 mg/kg). All three GGF2 treatment regimens resulted in significant improvements in cardiac physiology including EF, ESV and EDV and the effects were maintained for 10 days following termination of dosing. The studies presented here confirm GGF2 as the lead neuregulin compound and establish optimal dosing regimens for administering same.

As shown herein, the present studies establish the relative efficacy of GGF2 compared with published neuregulin fragments (Liu et al., 2006), initiate dose ranging and dose frequency studies, and determine if BSA excipient is required as previously reported.

### Results

Study 1—Treatment of rats with GGF2 at 0.625 mg/kg iv once per day (qday) resulted in significant improvement of cardiac function as shown here by changes in Ejection Fraction and Fractional Shortening. EGF-ld fragment did not result in the same degree of improvement. See Table 3 and FIG. 5.

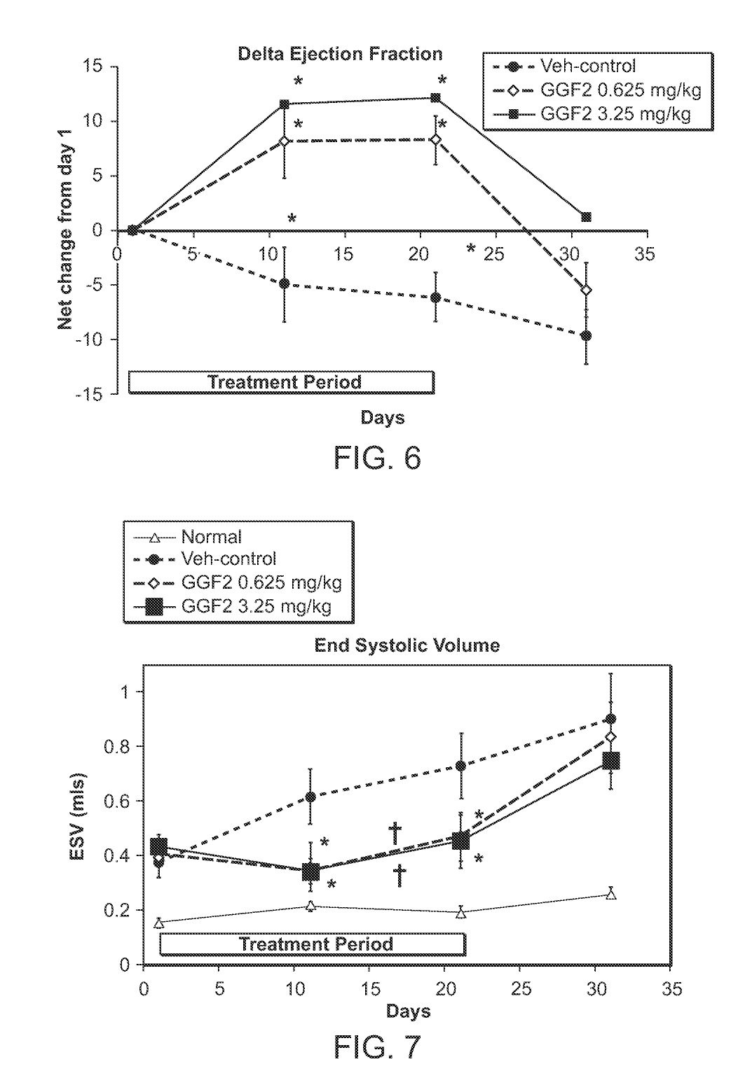

Study 2—Treatment of rats with GGF2 at 0.625 and 3.25 mg/kg iv qday resulted in significant improvement of cardiac function as shown here by changes in Ejection Fraction and Fractional Shortening. Significant improvements were also seen in end systolic and diastolic volumes during the treatment period. See Table 4 and FIGS. 6-7.

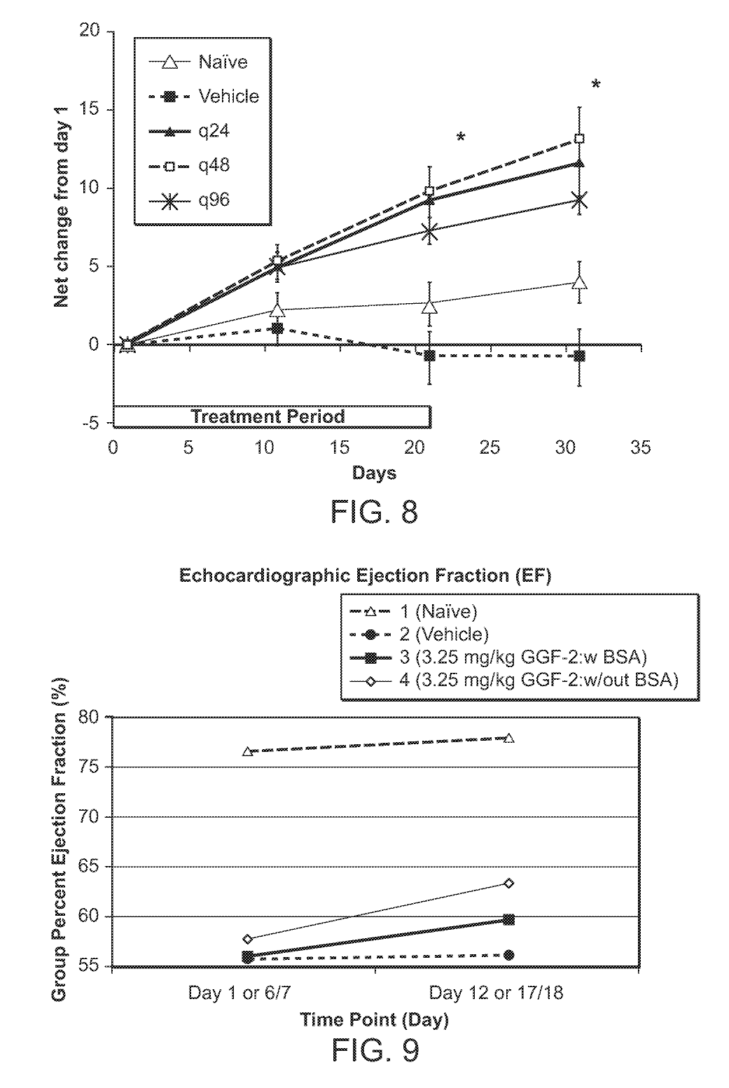

Study 3—Treatment of rats with GGF2 3.25 mg/kg iv once every 24, 48, or 96 hours (q24, 48 or 96 hours) resulted in significant improvement of cardiac function as shown here by changes in Ejection Fraction and Fractional Shortening. Significant improvements were also seen in end systolic and diastolic volumes during the treatment period. See Table 5 and FIG. 8.

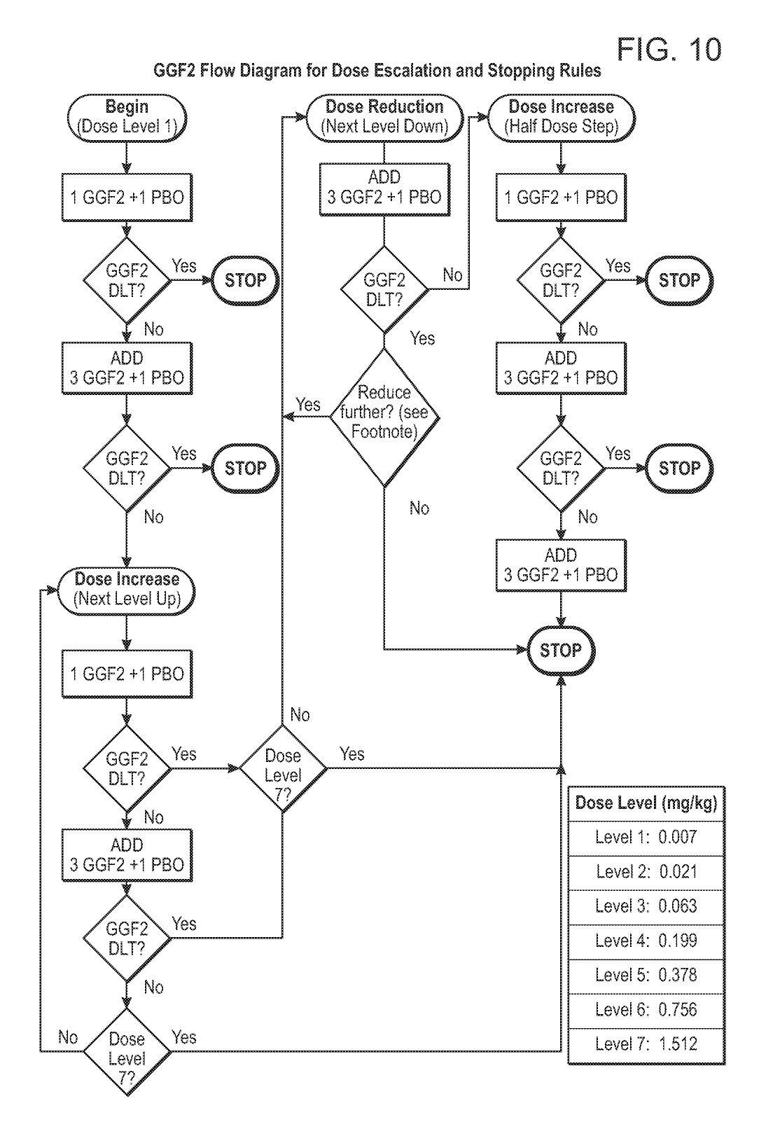

Previous reports (Liu et al) have shown that a carrier protein such as BSA is required for optimal neuregulin stability and activity. GGF2 has demonstrated stability without carriers such as BSA. This experiment was designed to test whether GGF2 is stable and active in a therapeutic regimen without BSA. After 10 days of treatment, both the BSA and non-BSA containing GGF2 formulations resulted in improvements in ejection fraction compared with vehicle controls similar to those seen in previous studies. It is, therefore, evident from this study that BSA or other carrier protein is not required in GGF2 formulations for the treatment of CHF. See Table 6 and FIG. 9.

TABLE 8
Pathology findings

| | | | Injection | |
|---|---|---|---|---|
| | Sciatic Nerve | | site −/− | |
| | Sheath Hyper- | Mammary | Skin | Cardiac |
| Dosing | plasia (NSH) | NSH | changes | effects |
| Daily s.c. | ++ | ++ | ++ | + |
| Daily i.v. | + | + | + | +/− |
| 48 hour interval i.v. | +/− | − | − | +/−− |
| 96 hour interval i.v. | − | − | − | − |

++ frequently present;
+ present;
+/− occasionally observed,
− rare or not observed

As shown in Table 8, intermittent dosing of GGF2 reduces side effects associated with supranormal levels of exogenously administered GGF2. The present inventors have discovered that this finding holds true irrespective of whether the GGF2 is administered intravenously or subcutaneously.

The hyperplasia and cardiac effects were sometimes seen with every other day dosing and were not seen with less frequent dosing.

### Example 4

### Human Clinical Safety and Tolerability Studies

A Phase 1, double-blind, placebo-controlled, dose escalation study to determine the safety, tolerability, pharmacokinetics and immunogenicity of single intravenous administrations of GGF2 in cohorts of patients with left ventricular dysfunction and symptomatic HF was undertaken. All patients had NYHA Class 2-3 HF, left ventricular ejection fraction (LVEF)<0.40 and had no significant renal or liver disease with an existing implantable defibrillator. An age-appropriate cancer screen was completed prior to enrollment. After informed consent, 40 patients with symptomatic HF were randomized (4:2) to GGF2 or placebo in 7 ascending dose cohorts from 0.007 to 1.5 mg/kg. Patients were observed in a hospital for 30 hours, then evaluated for adverse events (AEs) at 1, 2, 4, 12, and 24 weeks after infusion. AEs were graded using the Common Terminology Criteria for Adverse Events, version 4 (CTCAEv4).

TABLE 9
Study Synopsis
Study Synopsis

| | |
|---|---|
| Study Design | Phase-1, First-in-man |
| | Double-blind, placebo-controlled |
| Inclusion Criteria | LVEF 10-40% |
| | NYHA Class II-III |
| Methods | Single IV infusion |
| | 6 patients per cohort (4 GGF2:2 placebo) |
| | Escalating dose (0.007-1.512 mg/kg) |
| Evaluation | Safety |
| | Clinical |
| | ECG, holter monitor |
| | Echo |
| | BNP, Troponin |

Forty patients were enrolled in this study. Each of the patients satisfies the following diagnosis and main criteria for inclusion: 1) patient has systolic left ventricular dysfunction and symptomatic heart failure (Stage C; NYHA Class II-III), 2) patient is between 18 and 75 years of age, inclusive of the endpoints, and 3) patient has a left ventricular ejection fraction (LVEF) between 10-40%, inclusive of the endpoints). The patients enrolled in this study present chronic heart failures, meaning that the patient's condition has remained stable for at least 1, 2, 3, 4, 5, or 6 months. Stable or chronic heart failure is further characterized by the lack of increase or decrease in heart function and/or damage over a period of at least 1, 2, 3, 4, 5, or 6 months.

Patients who did not receive a placebo treatment received a dose of human recombinant GGF2. The dose of GGF2 was administered as an intravenous infusion with a fixed volume of 100 mL given over 15-20 minutes. As long as the total amount of drug given remains constant, e.g. a dose of GGF2 ranging from about 0.007 mg/kg to about 1.5 mg/kg, the dose of GGF2 may be administered as an intravenous infusion with any volume given over any length of time. The dose of GGF2 was preferably given in the morning. The starting dose of GGF2 was 0.007 mg/kg, which is approximately 1/30 of the NOAEL (no observed adverse level) identified from the most sensitive animal species toxicology study (or approximately 1/10 NOAEL applying the human equivalent dose factor of 3.1). The dose escalated in separate cohorts of six patients each, except for cohort seven. Dose escalation steps initially employed tripling of the dose in the initial three steps, then doubling the dose to a maximum dose of 1.512 mg/kg. The volume of administration remained fixed. The dose of GGF2 was administered as a single dose.

During escalation, in each of first six cohorts, four of the six patients received GGF2 and two of the six patients received placebo. In cohort seven, three patients received GGF2 and one received placebo. In each cohort (representing a dose level), the first two patients are randomized to GGF2 or placebo (1:1) and followed for 7 days for safety monitoring prior to initiating the other patients in the cohort. That is, if no drug-related dose-limiting toxicities are observed in the initial GGF2-treated patient, the four remaining patients in that cohort are randomized to receive GGF2 or placebo (3:1) and may be dosed at the same time. Dose escalation is based upon the occurrence of drug related toxicity. If there are no significant safety concerns by the time the last patient in each cohort reaches 28 days, the next dose level was initiated. FIG. 10 provides a schematic depiction of the decision tree for dose continuation and/or escalation before stopping treatment. GGF2 doses may begin at any level and progress to any level. With respect to dose-limiting toxicity (DLT), one or more of the following events that may have been at least possibly related to the study drug can trigger cessation of the treatment: 1) grade III toxicity or above (would encompass life threatening events), 2) liver function abnormalities as defined in the protocol, and 3) other events clinically judged to necessitate dose reduction or discontinuation of treatment.

Safety is assessed by review of toxicity profile, adverse events, vital signs (heart rate, respiration, systolic and diastolic BP), ECG changes, liver function tests, physical examination and laboratory parameters.

To evaluate the pharmacokinetics of GGF2 treatment, serial blood samples were collected prior to and at specified times for up to 24 hours following dosing of GGF2 for determination of GGF2 levels. A total of 8 blood samples were drawn.

To evaluate the effect of GGF2 treatment on cardiac function, the following techniques were used: electrocardiography (ECG or EKG); echocardiogram to determine ejection fraction (EF), end-diastolic volume (EDV), and/or end-systolic volume (ESV); evaluation of protein expression in either cardiac tissue or blood samples to determine levels of B-type Natiuretic Peptide (BNP), N-terminal B-type Natiuretic Peptide (NT BNP), and/or Troponin-I (TnI).

To evaluate the immunogenicity of GGF2 treatment, blood samples were taken for immunologic assessment at Baseline Day −1, Day 14, Day 28 and at 3 months post-dose.

Study Sequence: Patients were assessed on 8 occasions: Screening, Baseline Day −1, Day 1, Day 2, Day 8, Day 14, Day 28, and 3 months (study completion). The site also makes a 6-month post-treatment telephone call to the patient for medical follow-up (including adverse events).

Dosage Day Procedures: The following assessments are performed at Day 1 (patient is confined).

Pre-dose events included assessment of vital signs, e.g. pulse rate, respiration, blood pressure (supine and sitting), and oral temperature; recordation of weight; recordation of 12-lead ECG; collection of blood sample for PK assessment, glucose testing, and EPCs; assessment of selected injection sites and recordation of any skin abnormality; recordation of adverse events, potential toxicities and any changes in concomitant medications and therapies; and administration of treatment (double-blind GGF2 or placebo) in the contralateral arm, per the Site Instruction Manual.

Post-dose events include, but are not limited to, events include, but are not limited to, assessment of vital signs (e.g. pulse rate, respiration, blood pressure (supine and sitting), and oral temperature) at approximately 15 (±3) min and 30 (±3) min, then 1 hour (±10 min), 2 hours (±10 min), 4 hours (±10 min), 6 hours (±20 min), 8 hours (±20 min), and 12 hours (±20 min) after dosing. Post-dose events may include collection of blood samples for the following and documentation of time samples are drawn: PK/glucose assessments: 20 (±3) min, 45 (±3) min, and 90 (±10) min, then 3 hours (±10 min), 6 hours (±20 min), and 12 hours (±20 min) after dosing; EPCs: 20 (±3) min, 45 (±3) min, and 90 (±10) min, then 3 hours (±10 min) after dosing; and liver function tests:12 hours (±20 min) after dosing. Post-dose events may include recordation of local reactions at injection site at 30 (±3) min and 12 hours (±20 min) after dosing; recordation of 12-lead ECG at 30 (±3) min and 90 (±10) min, 3 hours (±10 min), 6 hours (±20 min), and 8 hours (±20 min) after dosing; recordation of adverse events and potential toxicities; and recordation any changes in concomitant medications or therapies.

Statistical methods: This was a Phase I single ascending dose study design with set numbers of patients per cohort and testing at ascending dose levels. No statistical justification has been applied to the number of patients required. Non-compartmental (model-independent) methods are used to derive pharmacokinetic parameters using individual patient plasma concentration-time data. PK parameters include the Cₘₐₓ, Tₘₐₓ, T_{1/2}, and the AUC.

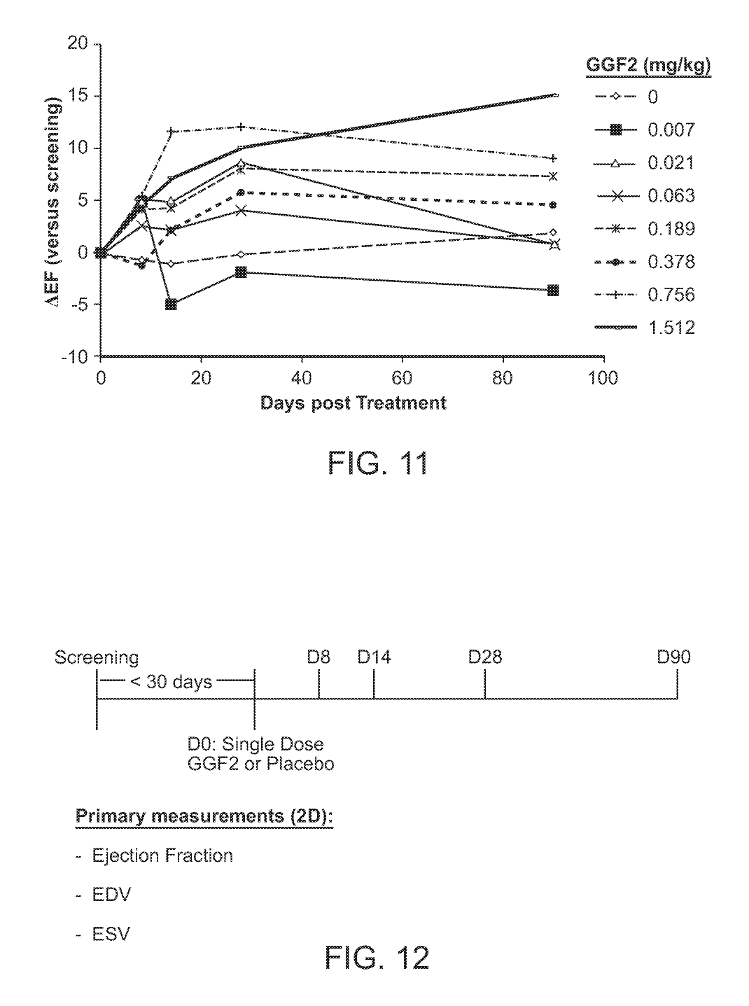

Results: Table 10 summarizes the demographic profile of patients enrolled in the study and their typical ongoing medications during the study period are shown in Table 11. There were no notable treatment effects of a single dose of GGF2 on hematologic, electrical, or the majority of biochemical safety laboratory testing performed. Serial echocardiographic measurements were obtained and the LVEF is displayed in FIG. 11. There was a dose related trend towards improved LVEF with increasing GGF2 doses. There were no adverse events leading to withdrawal of study drug. Treatment emergent adverse events (TEAEs) are shown in Table 12.

TABLE 10
Demographics of the Study Population

| | Total | | Placebo | | GGF2 | |
|---|---|---|---|---|---|---|
| | N = 40 | | N = 13 | | N = 27 | |
| Age | 57.4 | (9.8) | 54.7 | (13.2) | 58.6 | (7.7) |
| Male/Female | 33 (83%)/7 (17%) | | 12(92%)/1(8%) | | 21(78%)/6(22%) | |
| Caucasian | 36 | (90%) | 12 | (92%) | 24 | (89%) |
| African | 4 | (10%) | 1 | (8%) | 3 | (11%) |
| American | | | | | | |
| Weight (kg) | 93.8 | (21.2) | 102.2 | (23.1) | 89.8 | (19.4) |
| Duration of | 95.0 | (88.4) | 95.0 | (61.0) | 35.0 | (101.1) |
| HF (months) | | | | | | |
| Ischemic/ | 29(73%)/11(28%) | | 9(69%)/4(31%) | | 20(74%)/7(26%) | |
| Non- | | | | | | |
| ischemic | | | | | | |
| NYHA class | | | | | | |
| II | 24 | (60%) | 7 | (54%) | 17 | (63%) |
| III | 16 | (40%) | 6 | (46%) | 10 | (37%) |

All data is mean (standard deviation) except where number (percent) is indicated.
HF = heart failure,
NYHA = New York Heart Association

TABLE 11
Background medical therapy for all patient cohorts

| | | Drug | | | | | | | |
|---|---|---|---|---|---|---|---|---|---|
| | | Placebo | GGF2 (mg/kg) | | | | | | |
| | | Dose | | | | | | | |
| | | 0 | 0.007 | 0.021 | 0.063 | 0.189 | 0.378 | 0.756 | 1.512 |
| | | N | | | | | | | |
| | | 13 | 4 | 4 | 4 | 4 | 4 | 4 | 3 |
| | | M/F | | | | | | | |
| | | 12M/1F | 2M/2F | 4M/0F | 2M/2F | 3M/1F | 3M/1F | 4M/0F | 3M/0F |
| | | Average Age | | | | | | | |
| Drug Classes | ALL | 54.7 | 52.0 | 58.5 | 56.8 | 65.3 | 58.5 | 59.0 | 61.0 |
| Beta Blockers | 39 | 12 | 4 | 4 | 4 | 4 | 4 | 4 | 3 |
| ACE-Inhibitors/ARBs | 30 | 9 | 3 | 4 | 4 | 2 | 3 | 2 | 3 |
| Diuretics | 34 | 10 | 4 | 3 | 4 | 4 | 3 | 3 | 3 |
| Aldosterone Antagonists | 26 | 5 | 4 | 1 | 3 | 4 | 3 | 3 | 3 |
| Statins | 32 | 11 | 3 | 4 | 3 | 3 | 3 | 2 | 3 |
| Aspirin | 30 | 11 | 4 | 4 | 3 | 3 | 0 | 3 | 2 |
| Clopidogrel | 8 | 2 | 0 | 1 | 1 | 2 | 0 | 0 | 2 |
| (other antiplatelet) | | | | | | | | | |
| Coumadin/Heparin/ | 19 | 7 | 1 | 3 | 1 | 3 | 1 | 1 | 2 |
| Direct Thrombin | | | | | | | | | |
| Amiodarone/ | 9 | 4 | 0 | 2 | 0 | 1 | 1 | 1 | 0 |
| Other Antiarrhythmic | | | | | | | | | |
| Digoxin | 18 | 5 | 4 | 3 | 2 | 1 | 2 | 0 | 1 |
| Vasodilator | 5 | 2 | 1 | 0 | 0 | 1 | 0 | 1 | 0 |

TABLE 12
CTCAEv4 defined treatment emergent adverse events (TEAEs)

| | Placebo | 0.007 | 0.021 | 0.063 | 0.189 | 0.378 | 0.756 | 0.512 |
|---|---|---|---|---|---|---|---|---|
| GGF2 (mg/kg) Dose | (n = 13) | (n = 4) | (n = 4) | (n = 4) | (n = 4) | (n = 4) | (n = 4) | (n = 3) |
| Patients Any TEAEs | 5 | 4 | 4 | 2 | 4 | 4 | 4 | 3 |
| Total TEAEs | 6 | 12 | 7 | 4 | 16 | 13 | 13 | 10 |
| Nervous System - headache | | 2 | 2 | 1 | | 2 | 2 | |
| Nervous System - other | | | | | 3 | | | 2 |
| GI | 3 | 2 | | 1 | 2 | 2 | 3 | 2 |
| Administration Site | 2 | 1 | | 1 | 1 | 2 | 2 | 2 |
| Respiratory, Thoracic | | 3 | | | 1 | 2 | 1 | |
| Investigations and Bilirubin | | 1 | | | 2 | 2 | 1 | 0* |
| Vascular | | 1 | 1 | | 1 | | 2 | 1 |
| Infections | | 1 | | | 2 | 1 | | |
| Musculoskeletal | | | 2 | | 1 | | | 1 |
| Cardiac - Angina pectoris | | | 1 | | 1 | | | |
| Cardiac - HF | | | | | 1 | | | |
| Cardiac - flutter | | | | | | 1 | | |
| Metabolism and | 1 | | | | | | 2 | 1 |
| Renal and Urinary | | | | 1 | | | | 1** |
| Dermal | | 1 | | | 1 | | | |
| Ear and Labyrinth | | | | | | | | 1 |
| Eye | | | 1 | | | | | |
| Hepatobiliary - Hy's Law | | | | | | | | 1* |
| Procedural | | | | | | 1 | | |

| | |
|---|---|
| * | Defined Dose Limiting Toxicity: reversible elevation AST, ALT, Bilirubin |
| ** | Uroepithelial carcinoma in situ-investigation on-going |

Based on the data produced by this study, GGF2 appears safe and was generally well tolerated in a single ascending dose up to 0.756 mg/kg. The data indicate that LVEF may improve over a period from about 4 weeks to about 90 days following a single dose of GGF2. The LVEF may improve over a period of at least 4 weeks and/or at least 90 days. A dose limiting toxicity of transient liver dysfunction (Hy's law case) was seen at the highest dose (1.512 mg/kg) that resolved with observation after 8 days. The study demonstrates the safety and efficacy of GGF2 as a treatment for systolic heart failure.

### Example 5

### Clinical Evaluation of Cardiac Function in Symptomatic Heart Failure Patients

Methods: Single Infusion, Phase I, Dose Escalation Study of Glial Growth Factor 2 (GGF2) (See Table 9).

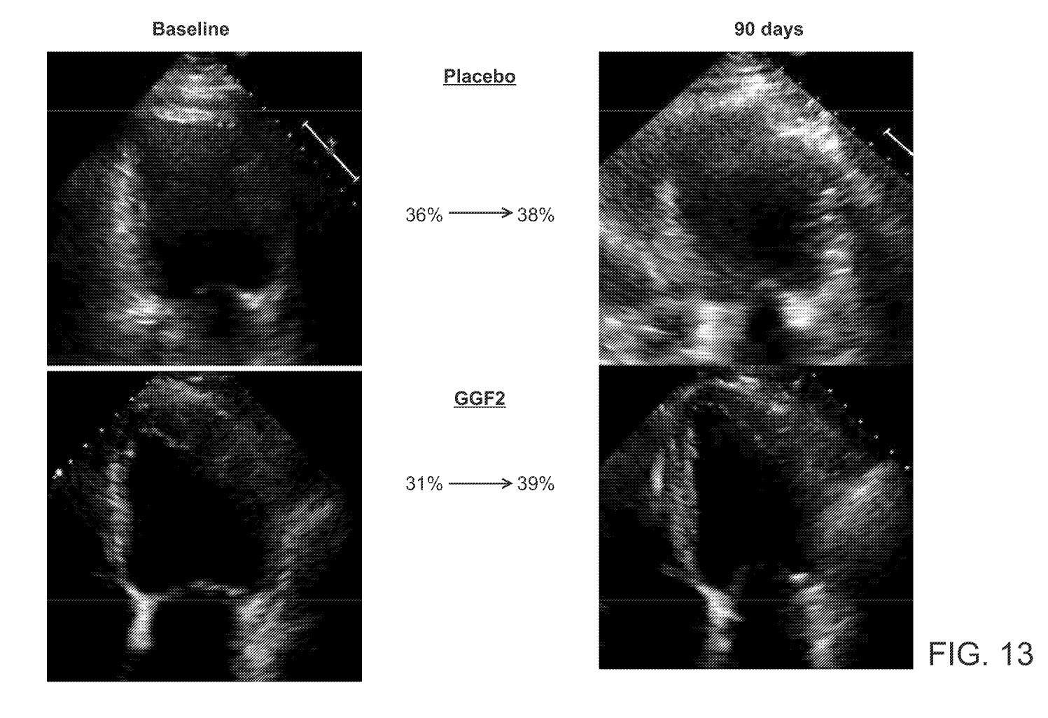

A diagram depicting the echocardiography protocol is shown in FIG. 12.

### Results

FIG. 11 demonstrates the change in Ejection Fraction (EF) as a function of the number of days following treatment with a single infusion of Glial Growth Factor 2 (GGF2) at varying dosages (provided in mg/kg).

FIG. 13 demonstrates the baseline and 90-days post-GGF2 treatment left ventricle ejection fractions (LVEFs) for the placebo versus highest dose of GGF2 (1.515 mg/kg).

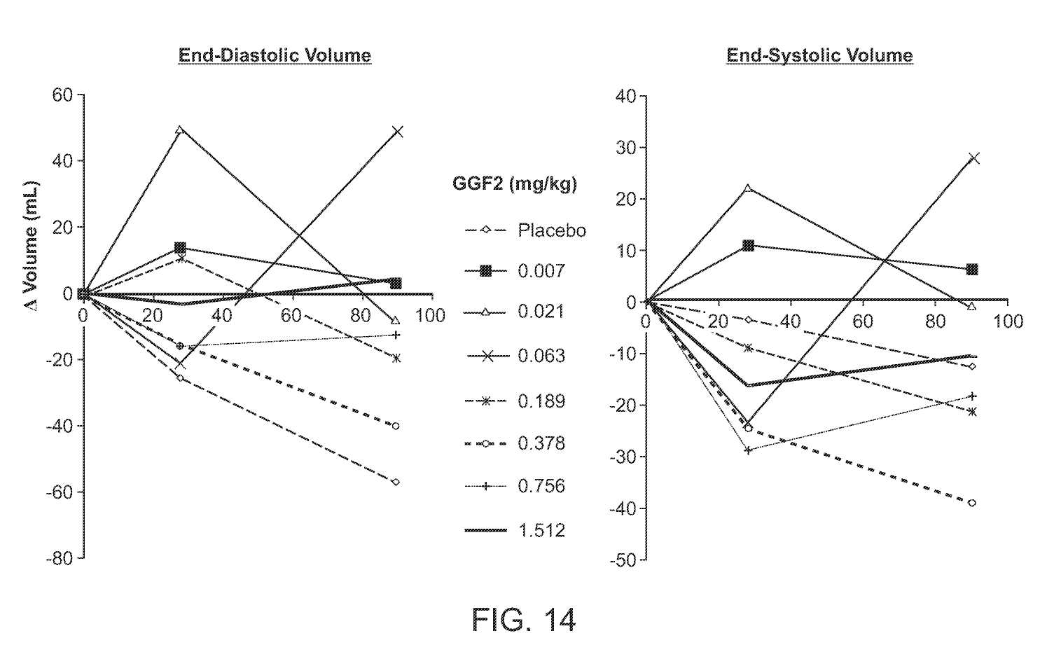

FIG. 14 demonstrates the mean change in dimensions (Δ volume) over time (days) following a single infusion of GGF2 or Placebo. The graph on left panel depicts the change in end-diastolic volume (EDV) as a function of time (measured in days post-treatment). The graph on right panel depicts the change in end-systolic volume (ESV) as a function of time (measured in days post-treatment).

Phase I of this study was completed with excellent safety and tolerability (Example 4). Data demonstrate improved cardiac function and a decrease in internal dimensions. Moreover, the data demonstrate a dose-dependent response to therapy at higher doses of GGF2 compared to placebo. Thus, a single dose or infusion of GGF2 improves left ventricle (LV) function over a period of 90 days compared to placebo.

### Example 6

### Evaluation of GGF2 Effects at Various Dose Levels with Bi-Weekly Administration on Left Ventricular Function in Rats following LAD Occlusion-Induced Myocardial Infarction (MI)

This study evaluates the effects of GGF2 treatment at various dose levels with bi-weekly (once every two week) administration on left ventricular (LV) function in rats with acute heart failure induced by LAD occlusion. A dose-dependent improvement in LV function was observed following bi-weekly intravenous administration of GGF2 when treatment was initiated 10-15 days following MI in rats.

Test system: Male naïve Sprague Dawley rats aged approximately 8 weeks and having a weight of approximately 250 grams at the time of surgery (175-200 grams at the time of arrival at the test facility) were used to evaluate left ventricular function (by, for example, echocardiograph) following LAD occlusion-induced heart failure.

Test and control articles: Rats were treated with either vehicle or GGF2. The vehicle comprised Acorda Formulation Buffer for GGF2. (20 mM histidine, 100 mM arginine, 100 mM sodium sulfate, 1% mannitol, pH 6.5). Treatment with GGF2 comprised a human recombinant form of GGF2 (rhGGF2) determined to have 96.0% purity by SEC-HPLC.

Experimental design: The overall design of the study is summarized in Table 13:

| | Animal | Surgical | | Dose Level | | | |
|---|---|---|---|---|---|---|---|
| Group | No. | Procedure | Treatment | (mg/kg) | Route | Regimen | In-life Duration |
| 1 | 12 | LAD | Vehicle | 0 | IV | Once every | ~18 weeks |
| | | Occlusion | | | | | |
| 2 | 14 | LAD | GGF2 | 3.5 | IV | Once every | ~18 weeks |
| | | Occlusion | | | | 2 weeks | |
| 3 | 14 | LAD | GGF2 | 1.75 | IV | Once every | ~18 weeks |
| | | Occlusion | | | | 2 weeks | |
| 4 | 14 | LAD | GGF2 | 0.88 | IV | Once every | ~18 weeks |
| | | Occlusion | | | | 2 weeks | |
| 5 | 14 | LAD | GGF2 | 0.35 | IV | Once every | ~18 weeks |
| | | Occlusion | | | | 2 weeks | |
| 6 | 9 | Naive | NA | NA | NA | NA | ~18 weeks |

Following receipt, naïve animals were weighed and monitored for a week. Animals were distributed into various surgical groups to minimize the differences between group mean body weights and group body weight variance. Animals were subjected to surgical left anterior descending coronary artery ligation (LAD occlusion) or not subjected to surgery (naïve). Seven to thirteen days following LAD occlusion, short axis left ventricular echocardiographic data were collected from each animal. 2-3 days following baseline imaging, rats were randomly assigned to treatment groups based on baseline LV function, and then subjected to the dosing paradigm, via the route and dose levels shown in Table 13 for 16 weeks (8 doses). A dosing volume of 1 mL/kg was used.

Animals were monitored for clinical signs of infection or post-operative pain/distress for 7 days prior to any left ventricular function assessment. The first echocardiographic evaluation took place approximately 7-13 days following LAD occlusion surgery.

Echocardiographic measurements were performed at approximately 7-13 days following surgery and once weekly (96 h) following initiation of dosing. Animals were euthanized at the conclusion of the study.

### Observations, Measurements, and Samples.

Clinical Observations: Animals were monitored at least once daily. General observations for morbidity, mortality, general animal health and behavior were recorded. All signs of clinical abnormality were recorded.

Body Weights: Body weights were obtained once weekly during the duration of the study. Individual animal body weight data are archived in the study records.

Tissue Preparation: Following euthanasia, hearts were harvested, and weighed.

LV Function: Left ventricular parameters were assessed by echocardiograph once weekly following initiation of dosing for the remainder of the in-life phase of the study. Mode data obtained from short axis views were used to derive LV parameters including: Ejection Fraction (% EF) and change in % EF, Fractional Shortening (% FS) and change in % FS, End Diastolic Volume (EDV), End Systolic Volume (ESV), and Left Ventricular Mass (LV Mass con).

Statistical Analysis of LV Parameters: Data were analyzed using Excel and GraphPad Prism (version 5.0). Mean LV parameter, standard deviation and standard error of the mean data was reported. The changes in various LV functional parameters relative to baseline values were analyzed. Statistical differences between group means during each separate treatment phase were assessed using ANOVA followed by a post-hoc test (e.g. Tukey or Dunnett's) at an α=0.05. Data obtained over time were subjected to repeated measures ANOVA followed by a Dunnett's and/or Tukey multiple comparison test at α=0.05.

### Results

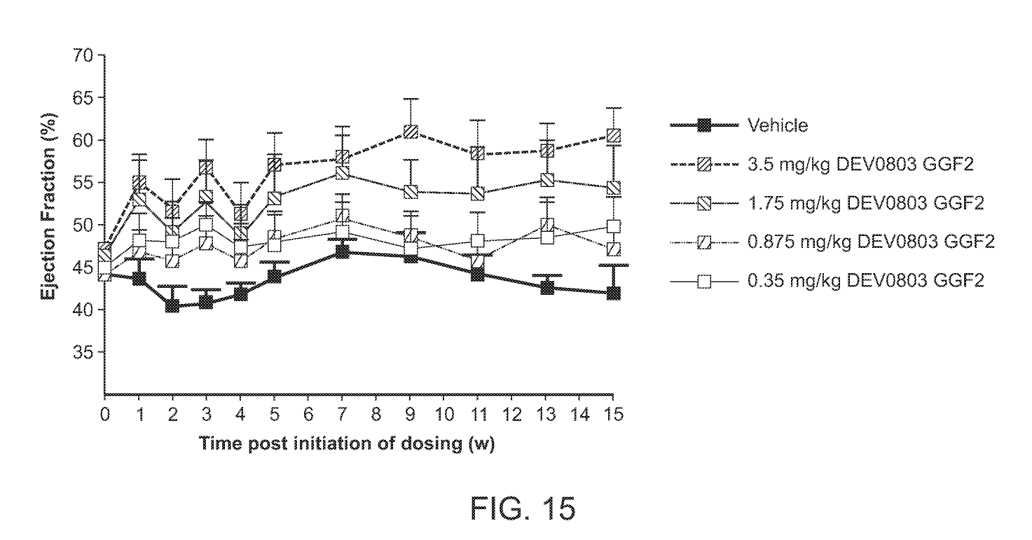

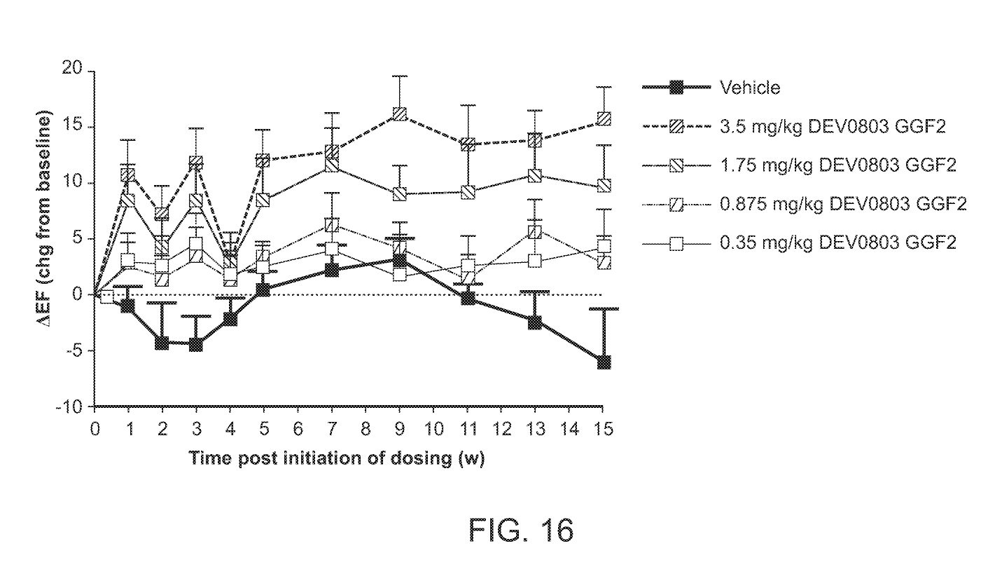

Echocardiographic changes: LV parameters were assessed by echocardiograph up to once weekly following initiation of dosing as described above. % EF and change in % EF data are shown in FIGS. 15 and 16. LAD occlusion significantly reduced % EF and change in % EF in all treatment groups over time compared to naïve animals (p<0.05). All intravenously administered GGF2 dose levels significantly improved the % EF and the change in % EF from baseline over time compared to vehicle-treated controls (p<0.05).

Similar to the effects seen on % EF and change in % EF over time, LAD occlusion significantly reduced % FS and change in % FS in all treatment groups compared to naïve controls (p<0.05).

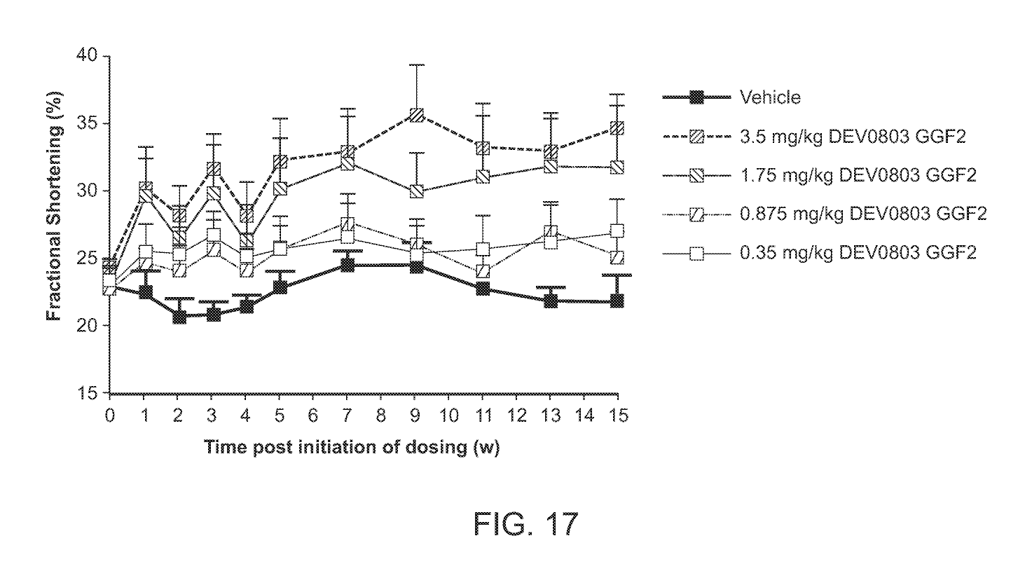

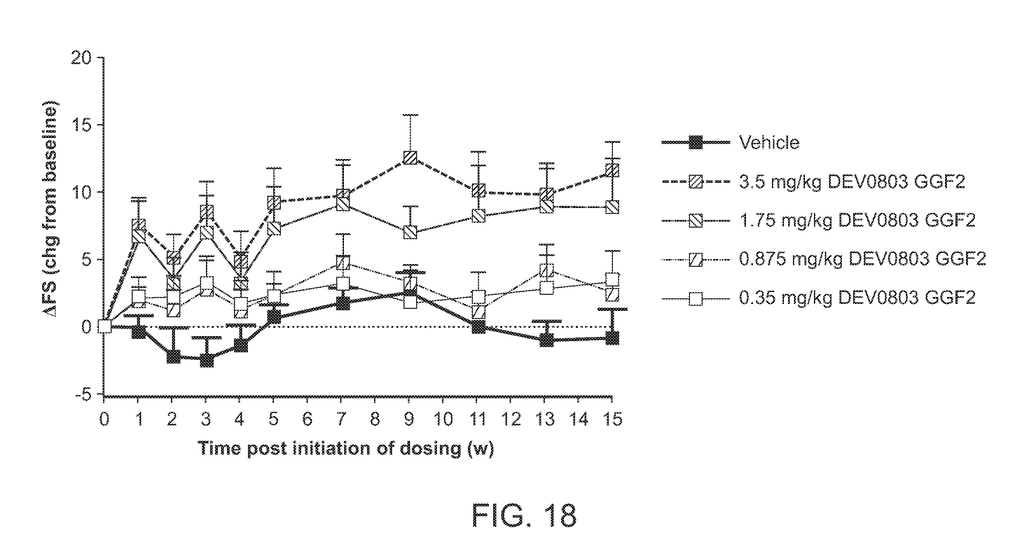

Intravenously administered GGF2 at all dose levels significantly improved the % FS and the change in % FS from baseline over time compared to vehicle-treated controls (p<0.05) change in % FS. See FIGS. 17 and 18, respectively.

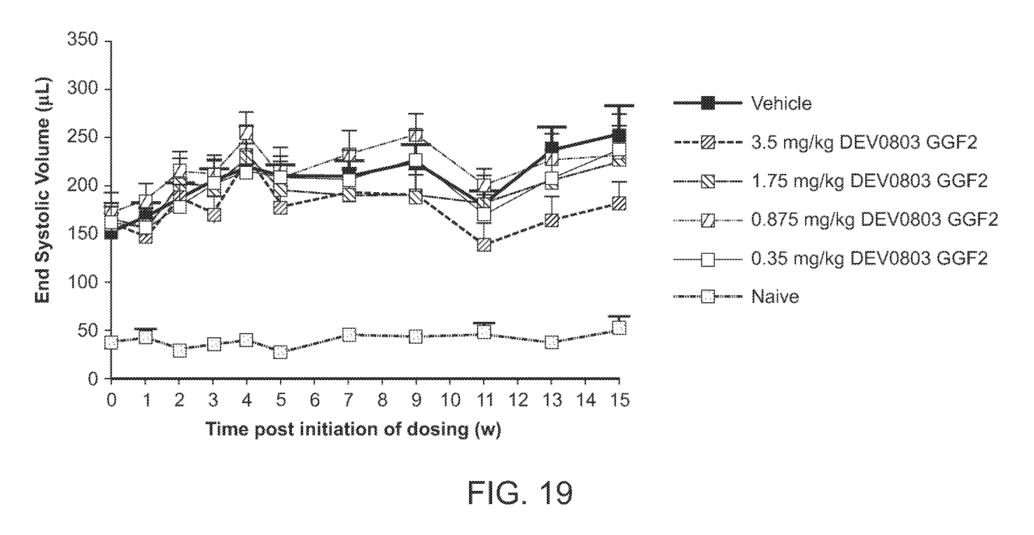

In addition to the above % EF and FS changes, LAD occlusion produced a significant increase in the ESV over time in all LAD-occlusion groups compared to naïve animals (p<0.05). Overall, administration of GGF2 led to a trend toward reduction in ESV compared to vehicle-treated controls and the value was significant at GGF2 administered at 3.5 mg/kg as shown in FIG. 19.

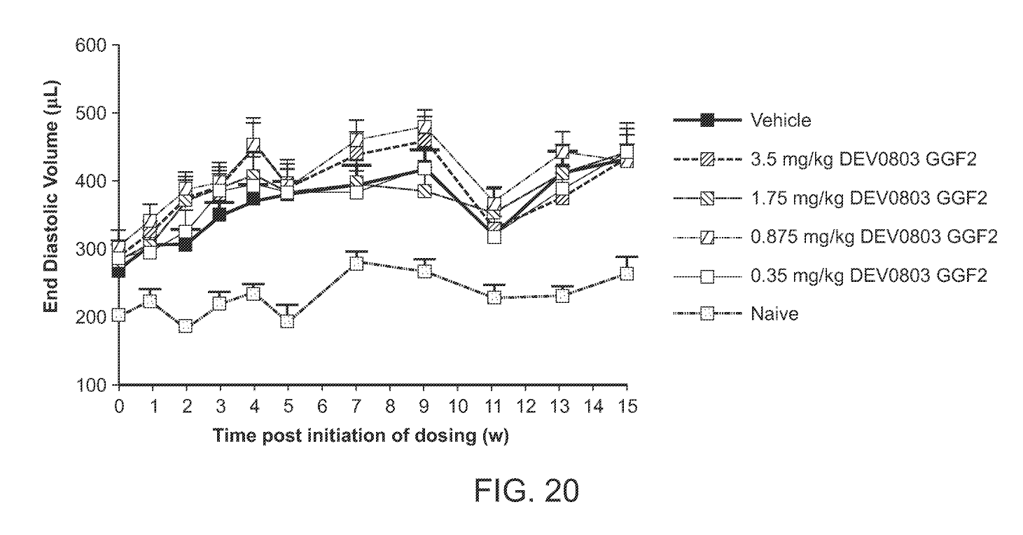

The effects of GGF2 on EDV are shown in FIG. 20. LAD occlusion produced a significant increase in the EDV over time in all LAD-occlusion groups compared to naïve animals (p<0.05). GGF2 treatment did not lead to any significant improvements in EDV compared to vehicle-treated controls.

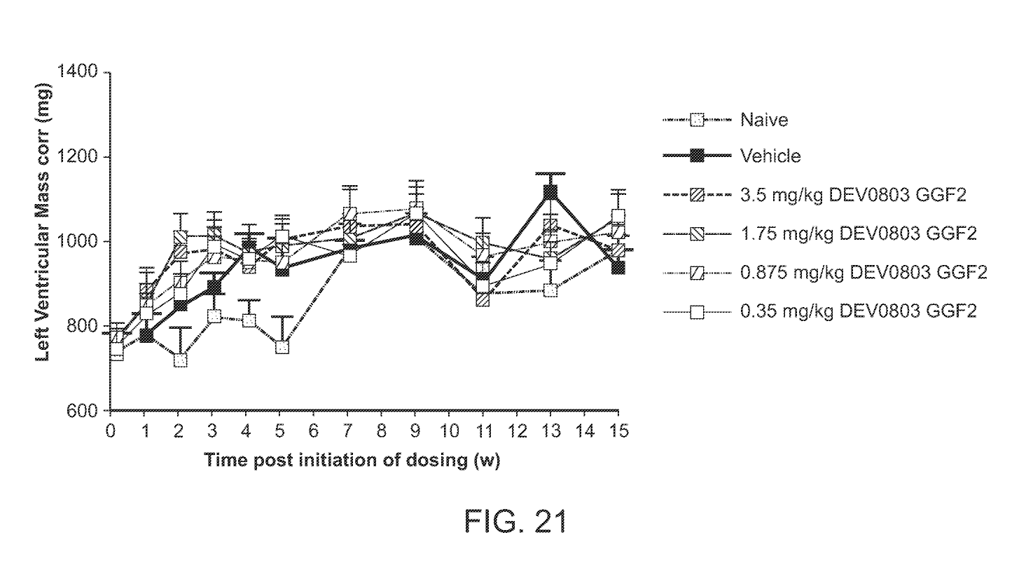

In addition, the effects of varying dose levels of GGF2 treatment on ventricular mass following LAD occlusion were evaluated. Overall, LV mass derived from echocardiographic assessment increased with body weight in all treatment groups. LAD occlusion led to a significant increase in LV mass compared to naïve animals in the post-infarction period. Overall, no obvious dose-dependent trends were observed following administration of GGF2 on LV mass compared to vehicle-treated controls (FIG. 21).

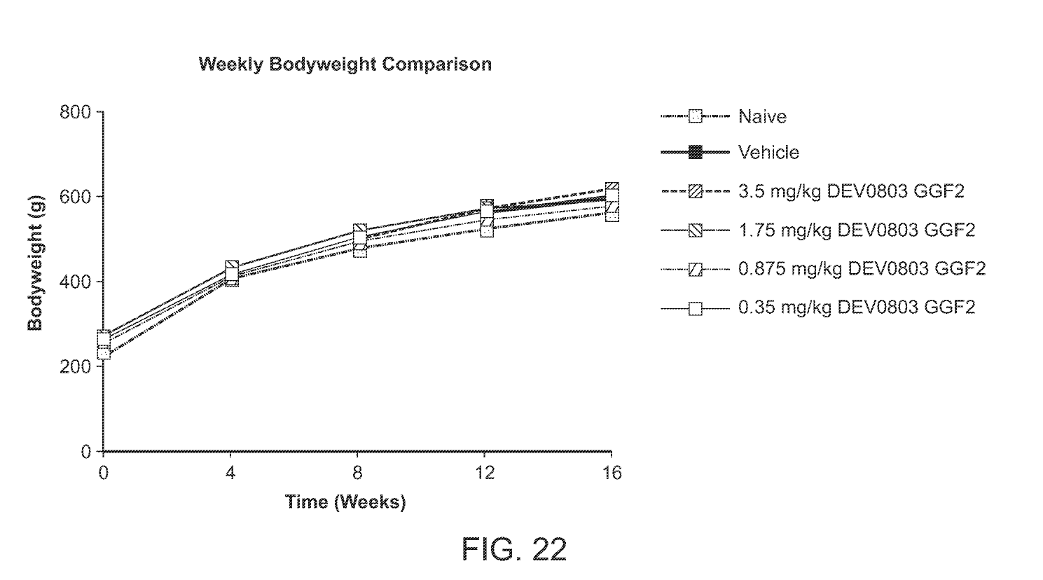

Body weight: Following LAD occlusion, all the treatment groups gained weight over time, but time-matched body weights were significantly lower compared to the naïve animals as shown in FIG. 22, presumably due to surgery. No significant differences in body weight over time were observed with GGF2 treatment compared to vehicle-treated controls.

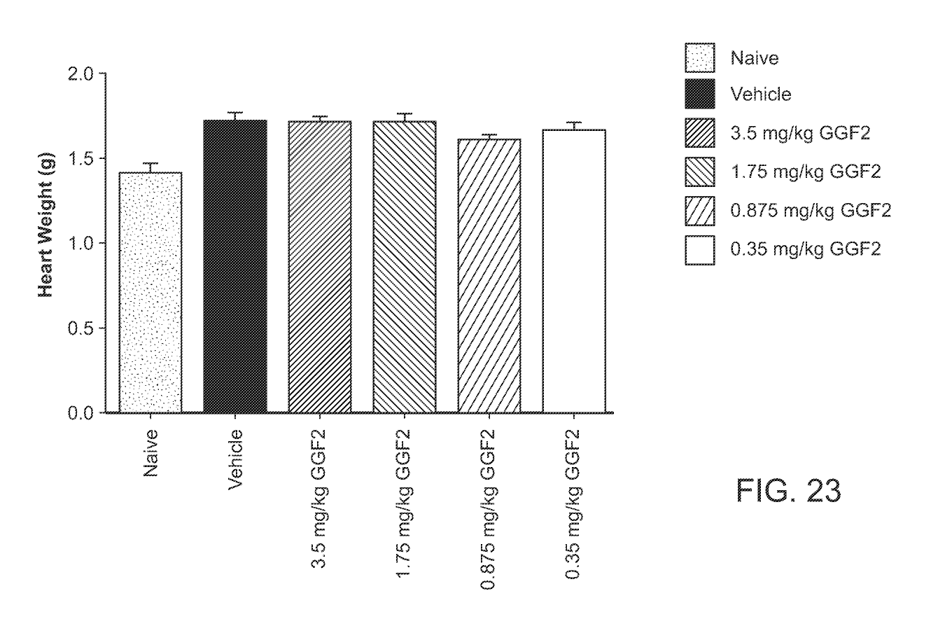

Heart weight: At the end of the study, heart weights were collected from all animals. The average heart weights for the various groups are shown in FIG. 23. Heart weights of LAD-occluded animals were significantly higher than that of the naïve animals. GGF2 treatment did not have any effects on heart weights.

Following LAD ligation, there was a significant decrease in left ventricular function as evidenced by significant reductions in % EF and % FS compared to naïve animals. There were also significant increases in the ESV and EDV in LAD animals compared to naïve animals. The compromised ventricular function was found to stable or be reduced slightly over the course of the study in vehicle-treated animals compared to the first time point.

Intravenous administration of GGF2 produced a dose-dependent improvement in cardiac function as evidenced by significant improvement in ejection fraction and fractional shortening over 16 weeks following LAD occlusion. There was a significant improvement in end systolic but not in end diastolic volume in GGF2-treated animals compared to vehicle-treated animals. All groups of animals gained weight during the course of the study; however, naïve animals had significantly greater weights compared to the LAD animals, presumably due to the effect of LAD-occlusion surgery. It can be concluded that bi-weekly administration of various dose levels of GGF2 via the intravenous route is effective in improving LV function after initiating treatment 10-15 days following MI in rats.

1. A method for treating or preventing heart failure in a subject in need thereof comprising administering to the subject a therapeutically effective amount of a peptide, wherein the peptide comprises an epidermal growth factor-like (EGF-like) domain, wherein the therapeutically effective amount is from about 0.005 mg/kg bodyweight to about 4 mg/kg bodyweight, and wherein the peptide is administered on a dosing interval of at least 24 hours.

2. The method of claim 1, wherein the peptide comprising an EGF-like domain is administered to the subject according to an escalating dosing regimen, said method comprising administering said peptide at a first therapeutically effective dose, and subsequently administering a second therapeutically effective dose, wherein the second dose is higher than the first dose.

3. The method of claim 1, wherein the therapeutically effective amount is from about 0.007 mg/kg bodyweight to about 1.5 mg/kg bodyweight.

4. The method of claim 1, wherein the therapeutically effective amount is selected from the group consisting of: about 0.007 mg/kg bodyweight, about 0.02 mg/kg bodyweight, about 0.06 mg/kg bodyweight, about 0.19 mg/kg bodyweight, about 0.38 mg/kg bodyweight, 0.76 mg/kg bodyweight, and about 1.51 mg/kg bodyweight.

5. The method of claim 1, wherein the dosing interval is at least 2 weeks.

6. The method of claim 5, wherein the therapeutically effective amount is about 0.35 mg/kg bodyweight to about 3.5 mg/kg bodyweight.

7. The method of claim 2, wherein the dosing regimen comprises the steps of:
a) administering an initial dose of the peptide in the range of about 0.005 mg/kg bodyweight to about 0.015 mg/kg bodyweight;
b) thereafter administering a second dose of the peptide that is 2-fold to 3-fold above the previous dose; and
c) repeating step b) until a maximum therapeutic dose is reached,
wherein the maximum therapeutic dose does not elicit an adverse event in the subject, and wherein the doses are administered on an interval of at least 24 hours.

8. The method of claim 7, wherein the maximum therapeutic dose is about 0.7 mg/kg bodyweight to about 1.5 mg/kg bodyweight.

9. The method of claim 1, wherein the peptide comprises glial growth factor 2 (GGF2) or a functional fragment thereof.

10. The method of claim 9, wherein the GGF2 or functional fragment thereof comprises the amino acid sequence of SEQ ID NO: 1 or SEQ ID NO: 2.

11. The method of claim 9, wherein the heart failure is chronic heart failure.

12. The method of claim 11, wherein the subject has suffered from chronic heart failure for at least 1 month prior to administration of the peptide.

13. The method of claim 9, wherein the subject suffers from class 2, 3, or 4 heart failure prior to administration of the peptide.

14. The method of claim 9, wherein the subject has a left ventricular ejection fraction of 40% or less or a preserved left ventricular ejection fraction prior to administration of the peptide.

15. The method of claim 9, wherein the therapeutically effective amount is sufficient to increase the left ventricular ejection fraction (LVEF), decrease the end systolic volume (ESV), decrease the end diastolic volume (EDV), increase the fractional shortening (FS), decrease the number of hospitalizations, increase exercise tolerance, decrease the number of occurrences of or the severity of mitral valve regurgitation, decrease dyspnea, decrease peripheral edema, or a combination thereof, in the subject.

16. The method of claim 15, wherein the increase in the left ventricular ejection fraction (LVEF), the decrease in the end systolic volume (ESV), the decrease in the end diastolic volume (EDV), the increase in the fractional shortening (FS), or combination thereof occurs within 90 days of the first administration of the peptide.

17. The method of claim 9, wherein the peptide is administered intravenously or subcutaneously.

18. The method of claim 9, further comprising administering a therapeutically effective amount of a benzodiazepine.

19.-20. (canceled)

21. The method of claim 5, wherein the dosing interval is one month, two months, three months, four months, five months, or six months.
